(19) United States
(12) Patent Application Publication
(10) Pub. No.: US 2019/0169181 A1
(43) Pub. Date: Jun. 6, 2019

(54) FUMAGILLOL COMPOUNDS AND METHODS OF MAKING AND USING SAME

(71) Applicant: Zafgen, Inc., Boston, MA (US)

(72) Inventors: Robert Zahler, Pennington, NJ (US); James E. Vath, Lynnfield, MA (US)

(21) Appl. No.: 16/324,692

(22) PCT Filed: Aug. 11, 2017
(86) PCT No.: PCT/US17/46485

§ 371 Date: Feb. 11, 2019

Related U.S. Application Data
(60) Provisional application No. 62/373,440, filed on Aug. 11, 2016.

(51) Int. Cl. C07D 413/14; C07D 417/14; C07D 405/14; A61P 3/04

(52) U.S. Cl. CPC C07D 413/14; A61P 3/04; C07D 405/14; C07D 417/14

(57) ABSTRACT

Disclosed herein, in part, are fumagillol compounds and methods of use in treating medical disorders, such as obesity. Pharmaceutical compositions and methods of making fumagillol compounds are provided. The compounds are contemplated to have activity against methionyl aminopeptidase 2.

### CROSS REFERENCE TO RELATED APPLICATIONS

This application is a national stage filing under 35 U.S.C. § 371 of PCT/US2017/046485, filed Aug. 11, 2017, which claims the benefit of, and priority to, U.S. Provisional Patent Application No. 62/373,440, filed Aug. 11, 2016, the contents of each of which is hereby incorporated by reference in their entirety.

### BACKGROUND

Over 1.1 billion people worldwide are reported to be overweight. Obesity is estimated to affect over 90 million people in the United States alone. Twenty-five percent of the population in the United States over the age of twenty is considered clinically obese. While being overweight or obese presents problems (for example restriction of mobility, discomfort in tight spaces such as theater or airplane seats, social difficulties, etc.), these conditions, in particular clinical obesity, affect other aspects of health, i.e., diseases and other adverse health conditions associated with, exacerbated by, or precipitated by being overweight or obese. The estimated mortality from obesity-related conditions in the United States is over 300,000 annually (O'Brien et al. Amer J Surgery (2002) 184:4S-8S; and Hill et al. (1998) Science, 280:1371).

There is no curative treatment for being overweight or obese. Traditional pharmacotherapies for treating an overweight or obese subject, such as serotonin and noradrenergic re-uptake inhibitors, noradrenergic re-uptake inhibitors, selective serotonin re-uptake inhibitors, intestinal lipase inhibitors, or surgeries such as stomach stapling or gastric banding, have been shown to provide minimal short-term benefits or significant rates of relapse, and have further shown harmful side-effects to patients.

MetAP2 encodes a protein that functions at least in part by enzymatically removing the amino terminal methionine residue from certain newly translated proteins such as glyceraldehyde-3-phosphate dehydrogenase (Warder et al. (2008) J Proteome Res 7:4807). Increased expression of the MetAP2 gene has been historically associated with various forms of cancer. Molecules inhibiting the enzymatic activity of MetAP2 have been identified and have been explored for their utility in the treatment of various tumor types (Wang et al. (2003) Cancer Res. 63:7861) and infectious diseases such as microsporidiosis, leishmaniasis, and malaria (Zhang et al. (2002) J. Biomed. Sci. 9:34). Notably, inhibition of MetAP2 activity in obese and obese-diabetic animals leads to a reduction in body weight in part by increasing the oxidation of fat and in part by reducing the consumption of food (Rupnick et al. (2002) Proc. Natl. Acad. Sci. USA 99:10730).

Such MetAP2 inhibitors may be useful as well for patients with excess adiposity and conditions related to adiposity including type 2 diabetes, hepatic steatosis, and cardiovascular disease (via e.g. ameliorating insulin resistance, reducing hepatic lipid content, and reducing cardiac workload). Accordingly, compounds capable of modulating MetAP2 are needed to address the treatment of obesity and related diseases as well as other ailments favorably responsive to MetAP2 modulator treatment.

### SUMMARY

The present disclosure provides, for example, compounds which may be modulators of MetAP2, and their use as medicinal agents, processes for their preparation, and pharmaceutical compositions containing them as an active ingredient both alone or in combination with other agents, as well as provides for their use as medicaments and/or in the manufacture of medicaments for the inhibition of MetAP2 activity in warm-blooded animals such as humans. In particular this disclosure relates to compounds useful for the treatment of obesity, type 2 diabetes, and other obesity-associated conditions. Also provided are pharmaceutical compositions comprising at least one disclosed compound and a pharmaceutically acceptable carrier.

For example, provided herein are compounds represented by Formula I and pharmaceutically acceptable salts, stereoisomers, esters, and prodrugs thereof:

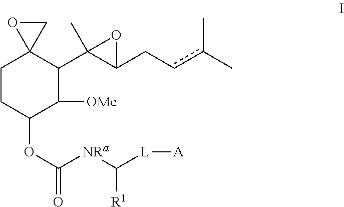

wherein [symbol] is a single or double bond; and A, L, R¹ and R^{a} are described herein.

Also provided herein is a pharmaceutically acceptable composition comprising a disclosed compound (e.g., of Formula I) and a pharmaceutically acceptable excipient.

Methods of treating and/or controlling obesity are contemplated herein, comprising administering to a patient in need thereof an effective amount of a disclosed compound (e.g., of Formula I). In an embodiment, a method of inducing weight loss in a patient in need thereof is provided, comprising administering to said patient an effective amount of a disclosed compound (e.g., of Formula I). In another embodiment, a method of substantially preventing weight gain in a patient in need thereof is provided comprising administering to said patient an effective amount of a disclosed compound (e.g., of Formula I).

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

The term “alkoxyalkyl” as used herein refers to a straight or branched alkyl group attached to oxygen, attached to a second straight or branched alkyl group (alkyl-O-alkyl-). Exemplary alkoxyalkyl groups include, but are not limited to, alkoxyalkyl groups in which each of the alkyl groups independently contains 1-6 carbon atoms, referred to herein as C₁₋₆alkoxy-C₁₋₆alkyl. Exemplary alkoxyalkyl groups include, but are not limited to methoxymethyl, 2-methoxyethyl, 1-methoxyethyl, 2-methoxypropyl, ethoxymethyl, 2-isopropoxyethyl, etc.

The term “alkyoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkenyloxy” used herein refers to a straight or branched alkenyl group attached to oxygen (alkenyl-O—). Exemplary alkenyloxy groups include, but are not limited to, groups with an alkenyl group of 3-6 carbon atoms, referred to herein as C₃₋₆alkenyloxy. Exemplary “alkenyloxy” groups include, but are not limited to allyloxy, butenyloxy, etc.

The term “alkynyloxy” used herein refers to a straight or branched alkynyl group attached to oxygen (alkynyl-O). Exemplary alkynyloxy groups include, but are not limited to, groups with an alkynyl group of 3-6 carbon atoms, referred to herein as C₃₋₆alkynyloxy. Exemplary alkynyloxy groups include, but are not limited to, propynyloxy, butynyloxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “cyano” as used herein refers to the radical —CN.

The term “cycloalkoxy” as used herein refers to a cycloalkyl group attached to oxygen (cycloalkyl-O—). Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc.

The terms “cycloalkyl” or a “carbocyclic group” as used herein refers to a saturated or partially unsaturated hydrocarbon group of, for example, 3-6, or 4-6 carbons, referred to herein as C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, oxadiazole, thiadiazole, pyridine, or pyrimidine, etc.

The terms “heterocyclyl” or “heterocyclic group” are art-recognized and refer to e.g. saturated or partially unsaturated, 4-10 membered ring structures, or e.g. 4-6 membered saturated ring structures, including bridged or fused rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran, etc.

The term “heterocyclyloxy” as used herein refers to a heterocyclyl group attached to oxygen (heterocyclyl-O—).

The term “heteroaryloxy” as used herein refers to a heteroaryl group attached to oxygen (heteroaryl-O—).

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment of obesity or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “F” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009. Where a particular compound is described or depicted, it is intended to encompass that chemical structure as well as tautomers of that structure.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the present disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as O-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a disclosed compound contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the present disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplício, et al., Molecules 2008, 13, 519 and references therein.

### I. Fumagillol Compounds

In an embodiment, the present disclosure provides compounds of Formula I:

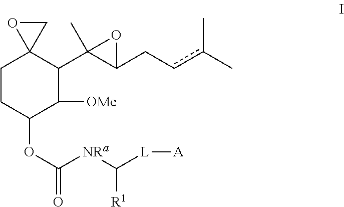

wherein:

[symbol] is a single or double bond;

R¹ is selected from the group consisting of hydrogen and C₁₋₆alkyl; wherein C₁₋₆alkyl may optionally be substituted by one, two, or more substituents each independently selected from the group consisting of halogen, cyano, hydroxyl, and N(R^{b}R^{c});

L is selected from the group consisting of a bond and —C(R^{L1}R^{L2})—;

A is a 5- or 6-membered monocyclic heteroaryl ring having one, two, or three heteroatoms each independently selected from O, S, or N; wherein A may be optionally substituted on an available carbon by a substituent independently selected for each occurrence from the group consisting of halogen, hydroxyl, C₁₋₆alkyl, C₁₋₆alkoxy, C₃₋₆cycloalkyl, phenyl, heterocyclyl, oxo, and —N(R^{b})(R^{c}); wherein C₁₋₆alkyl, C₁₋₆alkoxy, or C₃₋₆cycloalkyl may optionally be substituted by one, two, three or more substituents each independently selected from the group consisting of halogen, cyano, hydroxyl, and —N(R^{b})(R^{c}), and wherein A may be optionally substituted on an available nitrogen by R^{h};

R^{L1} and R^{L2} are each independently selected from the group consisting of hydrogen, halogen, hydroxyl, C₁₋₆alkyl, C₃₋₆cycloalkyl, and C₁₋₆alkoxy; wherein C₁₋₆alkyl, C₃₋₆cycloalkyl, and C₁₋₆alkoxy may optionally be substituted by one or more halogen atoms or a group selected from cyano or hydroxyl;

R^{a}, R^{b}, and R^{c} are each independently selected, for each occurrence, from the group consisting of hydrogen and C₁₋₆alkyl; wherein C₁₋₆alkyl may optionally be substituted by one or more substituents selected from the group consisting of halogen, cyano, oxo, and hydroxyl; or R^{b} and R^{c} may form, together with the nitrogen to which they are attached, a 4-6 membered heterocycle;

R^{h} is independently selected for each occurrence from the group consisting of hydrogen, C₁₋₆alkyl, C₂₋₆alkenyl, C₂₋₆alkynyl, C₃₋₆cycloalkyl, C₁₋₆alkyl-S(O)₂—, and C₁₋₆alkoxycarbonyl-, wherein C₁₋₆alkyl, C₂₋₆alkenyl, C₂₋₆alkynyl, C₃₋₆cycloalkyl, C₁₋₆alkyl-S(O)₂—, and C₁₋₆alkylcarbonyl- may optionally be substituted by one or more substituents selected from R^{P};

R^{P} is independently selected, for each occurrence, from the group consisting of halogen, hydroxyl, cyano, C₁₋₆alkoxy, and heterocyclyl; wherein heterocyclyl has one, two or three heteroatoms each independently selected from the group consisting of O, NR′ (wherein R′ is H or C₁₋₃alkyl) and S(O)_{w} (wherein w is 0, 1, or 2); wherein heterocyclyl may optionally be substituted by one, two or three substituents each independent selected from the group consisting of halogen, hydroxyl, and C₁₋₆alkyl; wherein C₁₋₆alkyl may optionally be substituted by one, two or three halogens;

or a pharmaceutically acceptable salt, stereoisomer, ester, or prodrug thereof.

In certain embodiments, A may be substituted with C₁₋₂alkyl, oxo, or —N(R^{b})(R^{c}).

In certain embodiments, R^{h} may be selected from the group consisting of hydrogen and C₁₋₆alkyl-R^{P}.

In an embodiment, R^{P} may be selected from the group consisting of:

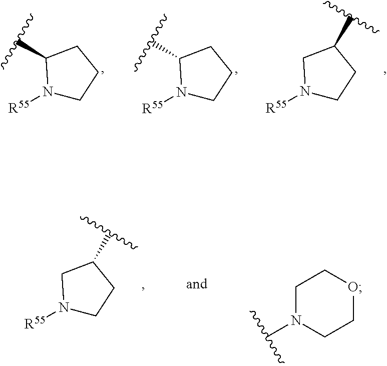

wherein R⁵⁵ is selected from the group consisting of hydrogen and C₁₋₂alkyl; wherein C₁₋₂alkyl may optionally be substituted by one or two halogens.

In certain embodiments, R^{a} may be hydrogen or methyl.

In some embodiments, L may be selected from the group consisting of a bond, —CH₂—, —CHMe-, and —CMe₂-. For example, L may be a bond.

In certain embodiments, R¹ may be selected from the group consisting of hydrogen, methyl, ethyl, isopropyl, and sec-butyl. For example, R¹ may be isopropyl or, e.g., methyl.

In certain embodiments, an A substituent may be selected from the group consisting of:

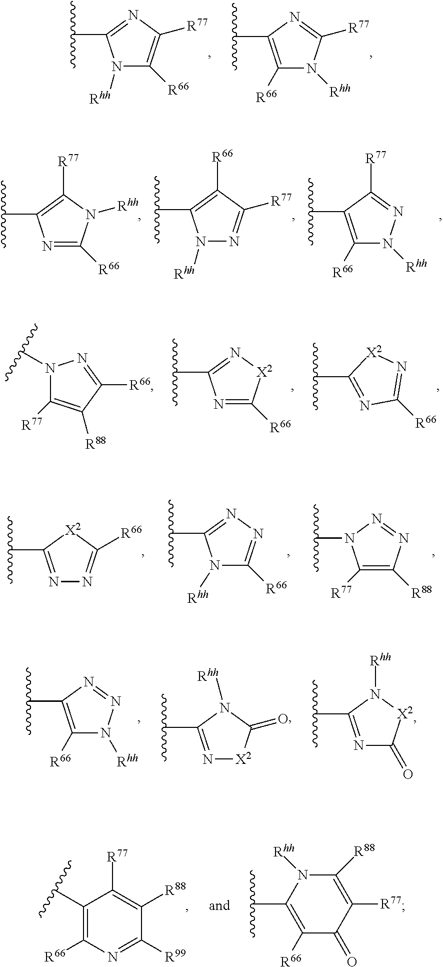

wherein X² independently for each occurrence is selected from the group consisting of O and S;

each R⁶⁶, R⁷⁷, R⁸⁸ and R⁹⁹ is independently selected for each occurrence from the group consisting of hydrogen, halogen, hydroxyl, C₁₋₂alkyl, heterocyclyl, and —NR^{b}R^{c}; wherein C₁₋₂alkyl may optionally be substituted by one, two or three substituents each independently selected from the group consisting of hydroxyl and halogen; and

R^{hh} is selected from the group consisting of hydrogen, C₁₋₆alkyl, and —C₁₋₃alkyl-heterocyclyl, wherein the heterocyclyl has one, two or three heteroatoms each independently selected from the group consisting of O, NR′ (wherein R′ is hydrogen or C₁₋₃alkyl) and S(O)_{w} (wherein w is 0, 1, or 2); and wherein heterocyclyl may optionally be substituted by a substituent selected from the group consisting of C₁₋₃alkyl (optionally substituted with 1-3 halogens), halogen, and hydroxyl.

For example, an A substituent may be selected from the group consisting of:

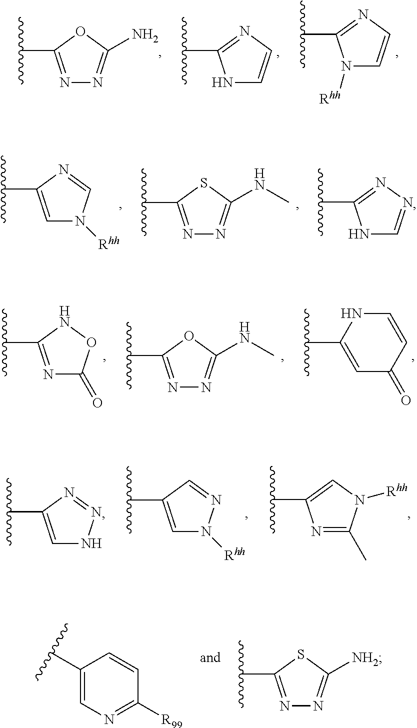

wherein

R^{hh} is hydrogen, C₁₋₆alkyl or —C₁₋₃alkyl-heterocyclyl, wherein heterocycyl may optionally be substituted by a substituent selected from the group consisting of C₁₋₃alkyl (optionally substituted by one, two, or three halogens), halogen, and hydroxyl;

and R⁹⁹ is heterocyclyl.

In certain embodiments, R^{hh} may be selected from the group consisting of:

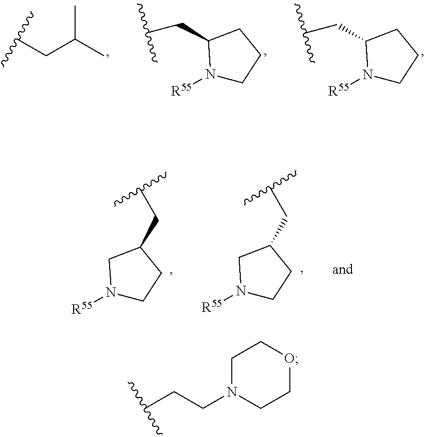

wherein R⁵⁵ is selected from the group consisting of hydrogen and C₁₋₂alkyl; wherein

C₁₋₂alkyl may optionally be substituted by 1 or 2 halogens.

For example, R^{hh} may be selected from the group consisting of:

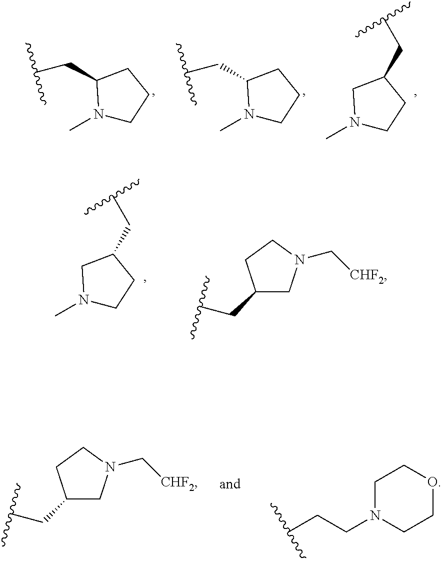

In certain embodiments, R⁹⁹ may be selected from the group consisting of:

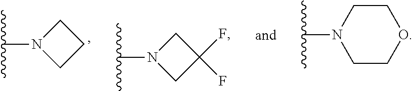

Also disclosed herein are compounds represented by:

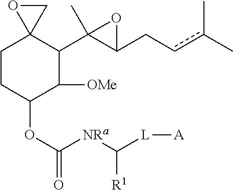

wherein:

[symbol] is a single or double bond;

R¹ is selected from the group consisting of hydrogen and C₁₋₆alkyl; wherein C₁₋₆alkyl may optionally be substituted by one, two, or more substituents each independently selected from the group consisting of halogen, cyano, hydroxyl, and N(R^{b}R^{c});

L is selected from the group consisting of a bond and —C(R^{L1}R^{L2})—;

A is selected from the group consisting of:

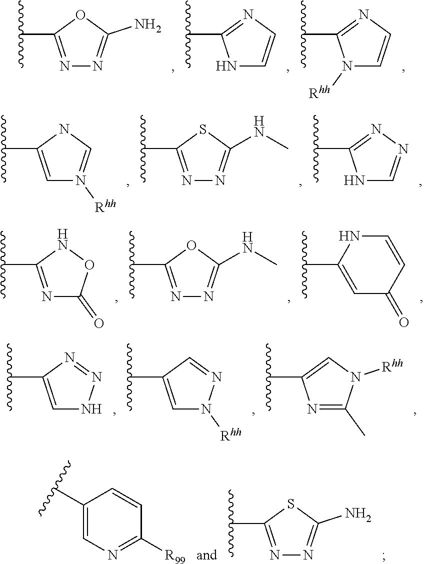

wherein

R^{hh} is hydrogen, C₁₋₆alkyl or —C₁₋₃alkyl-heterocyclyl, wherein heterocycyl may optionally be substituted by a substituent selected from the group consisting of C₁₋₃alkyl (optionally substituted by one, two, or three halogens), halogen, and hydroxyl;

R⁹⁹ is heterocyclyl;

R^{L1} and R^{L2} are each independently selected from the group consisting of hydrogen, halogen, hydroxyl, C₁₋₆alkyl, C₃₋₆cycloalkyl, and C₁₋₆alkoxy; wherein C₁₋₆alkyl, C₃₋₆cycloalkyl, and C₁₋₆alkoxy may optionally be substituted by one or more halogen atoms or a group selected from cyano or hydroxyl;

R^{a}, R^{b}, and R^{c} are each independently selected, for each occurrence, from the group consisting of hydrogen and C₁₋₆alkyl; wherein C₁₋₆alkyl may optionally be substituted by one or more substituents selected from the group consisting of halogen, cyano, oxo, and hydroxyl; or R^{b} and R^{c} may form, together with the nitrogen to which they are attached, a 4-6 membered heterocycle;

R^{h} is independently selected for each occurrence from the group consisting of hydrogen, C₁₋₆ alkyl, C₂₋₆ alkenyl, C₂₋₆alkynyl, C₃₋₆cycloalkyl, C₁₋₆alkyl-S(O)₂—, and C₁₋₆alkoxycarbonyl-, wherein C₁₋₆ alkyl, C₂₋₆alkenyl, C₂₋₆alkynyl, C₃₋₆cycloalkyl, C₁₋₆alkyl-S(O)₂—, and C₁₋₆alkylcarbonyl- may optionally be substituted by one or more substituents selected from R^{P};

R^{P} is independently selected, for each occurrence, from the group consisting of halogen, hydroxyl, cyano, C₁₋₆alkoxy, and heterocyclyl; wherein heterocyclyl has one, two or three heteroatoms each independently selected from the group consisting of O, NR′ (wherein R′ is H or C₁₋₃alkyl) and S(O)_{w} (wherein w is 0, 1, or 2); wherein heterocyclyl may optionally be substituted by one, two or three substituents each independent selected from the group consisting of halogen, hydroxyl, and C₁₋₆alkyl; wherein C₁₋₆alkyl may optionally be substituted by one, two or three halogens;

or a pharmaceutically acceptable salt, stereoisomer, ester, or prodrug thereof.

Also provided herein are compounds that may be selected from the group consisting of: (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-amino-1,3,4-oxadiazol-2-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(5-(methylamino)-1,3,4-oxadiazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-(methylamino)-1,3,4-oxadiazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1H-imidazol-2-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-2-yl)methyl)-1H-imidazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-2-yl)methyl)-1H-imidazol-2-yl)propyl)carbamate; (3R,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-(methylamino)-1,3,4-thiadiazol-2-yl)propyl)carbamate; (3R,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(5-(methylamino)-1,3,4-thiadiazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(4H-1,2,4-triazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(5-oxo-4,5-dihydro-1,2,4-oxadiazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-oxo-4,5-dihydro-1,2,4-oxadiazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(2-morpholinoethyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-(((S)-1-(2,2-difluoroethyl)pyrrolidin-3-yl)methyl)-1H-imidazol-4-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-(((R)-1-(2,2-difluoroethyl)pyrrolidin-3-yl)methyl)-1H-imidazol-4-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(4-oxo-1,4-dihydropyridin-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1H-1,2,3-triazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-3-yl)methyl)-1H-pyrazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-3-yl)methyl)-1H-pyrazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(2-methyl-1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(6-(3,3-difluoroazetidin-1-yl)pyridin-3-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(6-morpholinopyridin-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-3-methyl-1-(5-oxo-4,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-oxo-4,5-dihydro-1,2,4-oxadiazol-3-yl)propan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-oxo-4,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-oxo-2,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(4H-1,2,4-triazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-1-(5-amino-1,3,4-oxadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-(methylamino)-1,3,4-oxadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-3-methyl-1-(5-(methylamino)-1,3,4-thiadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-(methylamino)-1,3,4-thiadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-1-(5-amino-1,3,4-thiadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-amino-1,3,4-thiadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl (2-methyl-2-(1-(((R)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl (2-methyl-2-(4H-1,2,4-triazol-3-yl)propyl)carbamate; (3R,4S,5S,6R)-5-methoxy-4-((2R,3R)-2-methyl-3-(3-methylbut-2-enyl)oxiran-2-yl)-1-oxaspiro[2.5]octan-6-yl ((S)-1-(1-isobutyl-1H-imidazol-4-yl)-2-methylpropyl)(methyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-1-(6-(azetidin-1-yl)pyridin-3-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-isobutyl-1H-imidazol-4-yl)-2-methylpropyl)carbamate; and a pharmaceutically acceptable salt or stereoisomer thereof.

Procedures for making compounds described herein are provided below in the working examples and may be supplemented or substituted by procedures known to those of skill in the art. In the reactions described below, it may be necessary to protect reactive functional groups (such as hydroxyl, amino, thio or carboxyl groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed. (1999)). The deprotection step may be the final step in the synthesis such that the removal of protecting groups affords compounds of Formula I, as disclosed herein. Starting materials used in the working examples can be purchased or prepared by methods described in the chemical literature, or by adaptations thereof, using methods known by those skilled in the art. The order in which the steps are performed can vary depending on the groups introduced and the reagents used, but would be apparent to those skilled in the art.

Compounds of Formula I, or any of the intermediates described herein, can be further derivatized by using one or more standard synthetic methods known to those skilled in the art. Such methods can involve substitution, oxidation or reduction reactions. These methods can also be used to obtain or modify compounds of Formula I or any preceding intermediates by modifying, introducing or removing appropriate functional groups. Particular substitution approaches include alkylation, arylation, heteroarylation, acylation, thioacylation, halogenation, sulfonylation, nitration, formylation, hydrolysis and coupling procedures. These procedures can be used to introduce a functional group onto the parent molecule (such as the nitration or sulfonylation of aromatic rings) or to couple two molecules together (for example to couple an amine to a carboxylic acid to afford an amide; or to form a carbon-carbon bond between two heterocycles). For example, alcohol or phenol groups can be converted to ether groups by coupling a phenol with an alcohol in a solvent (such as tetrahydrofuran) in the presence of a phosphine (such as triphenylphosphine) and a dehydrating agent (such as diethyl, diisopropyl or dimethyl azodicarboxylate). Alternatively, ether groups can be prepared by deprotonation of an alcohol, using a suitable base (such as sodium hydride) followed by the addition of an alkylating agent (such as an alkyl halide or an alkyl sulfonate).

In another example, a primary or secondary amine can be alkylated using a reductive alkylation procedure. For example, the amine can be treated with an aldehyde and a borohydride (such as sodium triacetoxyborohydride, or sodium cyanoborohydride in a solvent (such as a halogenated hydrocarbon, for example dichloromethane, or an alcohol, for example ethanol) and, where necessary, in the presence of an acid (such as acetic acid).

In another example, hydroxy groups (including phenolic OH groups) can be converted into leaving groups, such as halogen atoms or sulfonyloxy groups (such as alkylsulfonyloxy, for example trifluoromethanesulfonyloxy, or aryl sulfonyloxy, for example p-toluenesulfonyloxy) using conditions known to those skilled in the art. For example, an aliphatic alcohol can be reacted with thionyl chloride in a halogenated hydrocarbon (such as dichloromethane) to afford the corresponding alkyl chloride. A base (such as triethylamine) can also be used in the reaction.

In another example, ester groups can be converted to the corresponding carboxylic acid by acid- or base-catalysed hydrolysis depending on the nature of the ester group. Acid catalysed hydrolysis can be achieved by treatment with an organic or inorganic acid (such as trifluoroacetic acid in an aqueous solvent, or a mineral acid such as hydrochloric acid in a solvent such as dioxane). Base catalysed hydrolysis can be achieved by treatment with an alkali metal hydroxide (such as lithium hydroxide in an aqueous alcohol, for example methanol).

In another example, aromatic halogen substituents in the compounds may be subjected to halogen-metal exchange by treatment with a base (such as a lithium base, for example n-butyl or t-butyl lithium) optionally at a low temperature (such as −78° C.) in a solvent (such as tetrahydrofuran) and the mixture may then be quenched with an electrophile to introduce a desired substituent. Thus, for example, a formyl group can be introduced by using dimethylformamide as the electrophile. Aromatic halogen substituents can also be subjected to palladium catalysed reactions to introduce groups such as carboxylic acids, esters, cyano or amino substituents.

In another example, an aryl, or heteroaryl ring substituted with an appropriate leaving group (such as a halogen or sulfonyl ester, for example a triflate) can undergo a palladium catalysed coupling reaction with a wide variety of substrates to form a carbon-carbon bond. For example, a Heck reaction can be used to couple such a ring system to an alkene (which may, or may not, be further substituted) by treatment with an organopalladium complex (such as tetrakis(triphenylphosphine)palladium(0), palladium (II) acetate or palladium (II) chloride) in the presence of a ligand (such as a phosphine, for example triphenylphosphine) in the presence of a base (such as potassium carbonate or a tertiary amine, for example, triethylamine), in an appropriate solvent (such as tetrahydrofuran or DMF), under appropriate conditions (such as heating to, for example, 50-120° C.). In another example, a Sonogashira reaction can be used to couple such a ring system to an alkyne (which may, or may not be further substituted) by treatment with a palladium complex (such as tetrakis(triphenylphosphine)palladium(0)) and a halide salt of copper (I) (such as copper (I) iodide), in the presence of a base (such as a potassium carbonate or a tertiary amine, for example, triethylamine), in an appropriate solvent (such as tetrahydrofuran or dimethylformamide), under appropriate conditions (such as heating to, for example, 50-120° C.). In another example, a Stille reaction can be used to couple such a ring system to an alkene, by treatment with an organotin compound (such as an alkynyltin or alkenyltin reagent, for example an alkenyltributylstannane) in the presence of a palladium complex (such as tetrakis(triphenylphosphine)palladium(0)), with, or without the presence of a salt (such as a copper (I) halide), in an appropriate solvent (such as dioxane or dimethylformamide), under appropriate conditions (such as heating to, for example, 50-120° C.).

Particular oxidation approaches include dehydrogenations and aromatization, decarboxylation and the addition of oxygen to certain functional groups. For example, aldehyde groups can be prepared by oxidation of the corresponding alcohol using conditions well known to those skilled in the art. For example, an alcohol can be treated with an oxidising agent (such as Dess-Martin periodinane) in a solvent (such as a halogenated hydrocarbon, for example dichloromethane). Alternative oxidising conditions can be used, such as treatment with oxalyl chloride and an activating amount of dimethylsulfoxide and subsequent quenching by the addition of an amine (such as triethylamine). Such a reaction can be carried out in an appropriate solvent (such as a halogenated hydrocarbon, for example dichloromethane) and under appropriate conditions (such as cooling below room temperature, for example to −78° C. followed by warming to room temperature). In another example, sulfur atoms can be oxidized to the corresponding sulfoxide or sulfone using an oxidising agent (such as a peroxy acid, for example 3-chloroperoxybenzoic acid) in an inert solvent (such as a halogenated hydrocarbon, for example dichloromethane) at around ambient temperature.

Particular reduction approaches include the removal of oxygen atoms from particular functional groups or saturation (or partial saturation) of unsaturated compounds including aromatic or heteroaromatic rings. For example, primary alcohols can be generated from the corresponding ester or aldehyde by reduction, using a metal hydride (such as lithium aluminium hydride or sodium borohydride in a solvent such as methanol). Alternatively, —CH₂OH groups can be generated from the corresponding carboxylic acid by reduction, using a metal hydride (such as lithium aluminium hydride in a solvent such as tetrahydrofuran). In another example, a nitro group may be reduced to an amine by catalytic hydrogenation in the presence of a metal catalyst (such as palladium on a solid support such as carbon) in a solvent (such as an ether, for example tetrahydrofuran, or an alcohol, such as methanol), or by chemical reduction using a metal (such as zinc, tin or iron) in the presence of an acid (such as acetic acid or hydrochloric acid). In a further example an amine can be obtained by reduction of a nitrile, for example by catalytic hydrogenation in the presence of a metal catalyst (such as palladium on a solid support such as carbon), or Raney nickel in a solvent (such as tetrahydrofuran) and under suitable conditions (such as cooling to below room temperature, for example to −78° C., or heating, for example to reflux).

Salts of compounds of Formula I can be prepared by the reaction of a compound of Formula I with an appropriate acid or base in a suitable solvent, or mixture of solvents (such as an ether, for example, diethyl ether, or an alcohol, for example ethanol, or an aqueous solvent) using conventional procedures. Salts of compound of Formula I can be exchanged for other salts by treatment using conventional ion-exchange chromatography procedures.

Where it is desired to obtain a particular enantiomer of a compound of Formula I, this may be produced from a corresponding mixture of enantiomers by employing any suitable conventional procedure for resolving enantiomers. For example, diastereomeric derivatives (such as salts) can be produced by reaction of a mixture of enantiomers of a compound of Formula I (such a racemate) and an appropriate chiral compound (such as a chiral base). The diastereomers can then be separated by any conventional means such as crystallization, and the desired enantiomer recovered (such as by treatment with an acid in the instance where the diastereomer is a salt). Alternatively, a racemic mixture of esters can be resolved by kinetic hydrolysis using a variety of biocatalysts (for example, see Patel Steroselective Biocatalysts, Marcel Decker; New York 2000).

In another resolution process a racemate of compounds of Formula I can be separated using chiral High Performance Liquid Chromatography. Alternatively, a particular enantiomer can be obtained by using an appropriate chiral intermediate in one of the processes described above. Chromatography, recrystallization and other conventional separation procedures may also be used with intermediates or final products where it is desired to obtain a particular geometric isomer of the present disclosure.

### II. Methods

Another aspect of the present disclosure provides methods of modulating the activity of MetAP2. Such methods comprise exposing said receptor to a compound described herein. In some embodiments, the compound utilized by one or more of the foregoing methods is one of the generic, subgeneric, or specific compounds described herein, such as a compound of Formula I. The ability of compounds described herein to modulate or inhibit MetAP2 can be evaluated by procedures known in the art and/or described herein. Another aspect of the present disclosure provides methods of treating a disease associated with expression or activity of MetAP2 in a patient.

In certain embodiments, the present disclosure provides a method of treating and/or controlling obesity, comprising administering to a patient in need thereof an effective amount of a disclosed compound.

In certain embodiments, the present disclosure provides a method of inducing weight loss in a patient in need thereof, comprising administering to said patient an effective amount of a disclosed compound.

In certain embodiments, the present disclosure provides a method of substantially preventing weight gain in a patient in need thereof, comprising administering to said patient an effective amount of a disclosed compound.

In certain embodiments, the patient is a human.

In certain embodiments, the patient is a cat or dog.

In certain embodiments, the patient has a body mass index greater than or equal to about 30 kg/m² before the administration.

In certain embodiments, administering a disclosed compound may comprise subcutaneous administration. In certain embodiments, administering a disclosed compound may comprise intravenous administration.

Provided methods of treatment may include administering a disclosed compound once, twice, or three times daily; about every other day (e.g. every 2 days); twice weekly (e.g. every 3 days, every 4 days, every 5 days, every 6 days, or e.g. administered with an interval of about 2 to about 3 days between doses); once weekly; three times weekly; every other week; twice monthly; once a month; every other month; or even less often.

In certain embodiments, a method disclosed herein further comprises administering said compound in an amount sufficient to establish inhibition of intracellular MetAP2 effective to increase thioredoxin production in the patient and to induce multi organ stimulation of anti-obesity processes in the subject.

In certain embodiments, the method comprises administering said compound in an amount insufficient to reduce angiogenesis in the patient.

Other contemplated methods of treatment include method of treating or ameliorating an obesity-related condition or co-morbidity, by administering a compound disclosed herein to a subject. For example, contemplated herein are methods for treating type 2 diabetes in a patient in need thereof.

Exemplary co-morbidities include cardiac disorders, endocrine disorders, respiratory disorders, hepatic disorders, skeletal disorders, psychiatric disorders, metabolic disorders, and reproductive disorders.

Exemplary cardiac disorders include hypertension, dyslipidemia, ischemic heart disease, cardiomyopathy, cardiac infarction, stroke, venous thromboembolic disease and pulmonary hypertension. Exemplary endocrine disorders include type 2 diabetes and latent autoimmune diabetes in adults. Exemplary respiratory disorders include obesity-hypoventilation syndrome, asthma, and obstructive sleep apnea. An exemplary hepatic disorder is nonalcoholic fatty liver disease. Exemplary skeletal disorders include back pain and osteoarthritis of weight-bearing joints. Exemplary metabolic disorders include Prader-Willi Syndrome and polycystic ovary syndrome. Exemplary reproductive disorders include sexual dysfunction, erectile dysfunction, infertility, obstetric complications, and fetal abnormalities. Exemplary psychiatric disorders include weight-associated depression and anxiety.

In particular, in certain embodiments, the present disclosure provides a method of treating one or more of the above medical indications comprising administering to a subject in need thereof a therapeutically effective amount of a compound described herein, such as a compound of Formula I.

Obesity or reference to “overweight” refers to an excess of fat in proportion to lean body mass. Excess fat accumulation is associated with increase in size (hypertrophy) as well as number (hyperplasia) of adipose tissue cells. Obesity is variously measured in terms of absolute weight, weight:height ratio, distribution of subcutaneous fat, and societal and esthetic norms. A common measure of body fat is Body Mass Index (BMI). The BMI refers to the ratio of body weight (expressed in kilograms) to the square of height (expressed in meters). Body mass index may be accurately calculated using either of the formulas: weight(kg)/height²(m²) (SI) or 703×weight(lb)/height²(in²) (US).

In accordance with the U.S. Centers for Disease Control and Prevention (CDC), an overweight adult has a BMI of 25 kg/m² to 29.9 kg/m², and an obese adult has a BMI of 30 kg/m² or greater. A BMI of 40 kg/m² or greater is indicative of morbid obesity or extreme obesity. Obesity can also refer to patients with a waist circumference of about 102 cm for males and about 88 cm for females. For children, the definitions of overweight and obese take into account age and gender effects on body fat. Patients with differing genetic background may be considered “obese” at a level differing from the general guidelines, above.

The compounds of the present disclosure also are useful for reducing the risk of secondary outcomes of obesity, such as reducing the risk of left ventricular hypertrophy. Methods for treating patients at risk of obesity, such as those patients who are overweight, but not obese, e.g. with a BMI of between about 25 and 30 kg/m², are also contemplated. In certain embodiments, a patient is a human.

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male.

Methods of determining whether a subject is overweight or obese that account for the ratio of excess adipose tissue to lean body mass involve obtaining a body composition of the subject. Body composition can be obtained by measuring the thickness of subcutaneous fat in multiple places on the body, such as the abdominal area, the subscapular region, arms, buttocks and thighs. These measurements are then used to estimate total body fat with a margin of error of approximately four percentage points. Another method is bioelectrical impedance analysis (BIA), which uses the resistance of electrical flow through the body to estimate body fat. Another method is using a large tank of water to measure body buoyancy. Increased body fat will result in greater buoyancy, while greater muscle mass will result in a tendency to sink.

In another aspect, the present disclosure provides methods for treating an overweight or obese subject involving determining a level of at least one biomarker related to being overweight or obese in the subject, and administering an effective amount of a disclosed compound to achieve a target level in the subject. Exemplary biomarkers include body weight, Body Mass Index (BMI), Waist/Hip ratio WHR, plasma adipokines, and a combination of two or more thereof.

In certain embodiments, the compound utilized by one or more of the foregoing methods is one of the generic, subgeneric, or specific compounds described herein, such as a compound of Formula I.

The compounds of the present disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound of this present disclosure may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. A suitable treatment period can be, for example, at least about one week, at least about two weeks, at least about one month, at least about six months, at least about 1 year, or indefinitely. A treatment period can terminate when a desired result, for example a weight loss target, is achieved. A treatment regimen can include a corrective phase, during which dose sufficient to provide reduction of weight is administered, and can be followed by a maintenance phase, during which a e.g. a lower dose sufficient to prevent weight gain is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain body weight in subjects whose body weight has been previously controlled by other means, including diet and exercise, bariatric procedures such as bypass or banding surgeries, or treatments employing other pharmacological agents.

### III. Pharmaceutical Compositions and Kits

Another aspect of the present disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more disclosed compounds, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the present disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present invention.

Advantageously, the present disclosure also provides kits for use by e.g. a consumer in need of weight loss. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent. For example, in addition to being overweight or obese, a subject or patient can further have overweight- or obesity-related co-morbidities, i.e., diseases and other adverse health conditions associated with, exacerbated by, or precipitated by being overweight or obese. Contemplated herein are disclosed compounds in combination with at least one other agent that has previously been shown to treat these overweight- or obesity-related conditions.

For example, Type II diabetes has been associated with obesity. Certain complications of Type II diabetes, e.g., disability and premature death, can be prevented, ameliorated, or eliminated by sustained weight loss (Astrup, A. Pub Health Nutr (2001) 4:499-5 15). Agents administered to treat Type II diabetes include sulfonylureas (e.g., Chlorpropamide, Glipizide, Glyburide, Glimepiride); meglitinides (e.g., Repaglinide and Nateglinide); biguanides (e.g., Metformin); thiazolidinediones (Rosiglitazone, Troglitazone, and Pioglitazone); dipeptidylpeptidase-4 inhibitors (e.g., Sitagliptin, Vildagliptin, and Saxagliptin); glucagon-like peptide-1 mimetics (e.g., Exenatide and Liraglutide); and alpha-glucosidase inhibitors (e.g., Acarbose and Miglitol.

Cardiac disorders and conditions, for example hypertension, dyslipidemia, ischemic heart disease, cardiomyopathy, cardiac infarction, stroke, venous thromboembolic disease and pulmonary hypertension, have been linked to overweight or obesity. For example, hypertension has been linked to obesity because excess adipose tissue secretes substances that are acted on by the kidneys, resulting in hypertension. Additionally, with obesity there are generally higher amounts of insulin produced (because of the excess adipose tissue) and this excess insulin also elevates blood pressure. A major treatment option of hypertension is weight loss. Agents administered to treat hypertension include Chlorthalidone; Hydrochlorothiazide; Indapamide, Metolazone; loop diuretics (e.g., Bumetanide, Ethacrynic acid, Furosemide, Lasix, Torsemide); potassium-sparing agents (e g, Amiloride hydrochloride, benzamil, Spironolactone, and Triamterene); peripheral agents (e.g., Reserpine); central alpha-agonists (e.g., Clonidine hydrochloride, Guanabenz acetate, Guanfacine hydrochloride, and Methyldopa); alpha-blockers (e.g., Doxazosin mesylate, Prazosin hydrochloride, and Terazosin hydrochloride); beta-blockers (e.g., Acebutolol, Atenolol, Betaxolol, Bisoprolol fumarate, Carteolol hydrochloride, Metoprolol tartrate, Metoprolol succinate, Nadolol, Penbutolol sulfate, Pindolol, Propranolol hydrochloride, and Timolol maleate); combined alpha- and beta-blockers (e.g., Carvedilol and Labetalol hydrochloride); direct vasodilators (e.g., Hydralazine hydrochloride and Minoxidil); calcium antagonists (e.g., Diltiazem hydrochloride and Verapamil hydrochloride); dihydropyridines (e.g., Amlodipine besylate, Felodipine, Isradipine, Nicardipine, Nifedipine, and Nisoldipine); ACE inhibitors (benazepril hydrochloride, Captopril, Enalapril maleate, Fosinopril sodium, Lisinopril, Moexipril, Quinapril hydrochloride, Ramipril, Trandolapril); Angiotensin II receptor blockers (e.g., Losartan potassium, Valsartan, and Irbesartan); Renin inhibitors (e.g., Aliskiren); and combinations thereof. These compounds are administered in regimens and at dosages known in the art.

Carr et al. (The Journal of Clinical Endocrinology & Metabolism (2004) Vol. 89, No. 6 2601-2607) discusses a link between being overweight or obese and dyslipidemia. Dyslipidemia is typically treated with statins. Statins, HMG-CoA reductase inhibitors, slow down production of cholesterol in a subject and/or remove cholesterol buildup from arteries. Statins include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin. These compounds are administered in regimens and at dosages known in the art. Eckel (Circulation (1997) 96:3248-3250) discusses a link between being overweight or obese and ischemic heart disease. Agents administered to treat ischemic heart disease include statins, nitrates (e.g., Isosorbide Dinitrate and Isosorbide Mononitrate), beta-blockers, and calcium channel antagonists. These compounds are administered in regimens and at dosages known in the art.

Wong et al. (Nature Clinical Practice Cardiovascular Medicine (2007) 4:436-443) discusses a link between being overweight or obese and cardiomyopathy. Agents administered to treat cardiomyopathy include inotropic agents (e.g., Digoxin), diuretics (e.g., Furosemide), ACE inhibitors, calcium antagonists, anti-arrhythmic agents (e.g., Sotolol, Amiodarone and Disopyramide), and beta-blockers. These compounds are administered in regimens and at dosages known in the art. Yusef et al. (Lancet (2005) 366(9497):1640-1649) discusses a link between being overweight or obese and cardiac infarction. Agents administered to treat cardiac infarction include ACE inhibitors, Angiotensin II receptor blockers, direct vasodilators, beta blockers, anti-arrhythmic agents and thrombolytic agents (e.g., Alteplase, Retaplase, Tenecteplase, Anistreplase, and Urokinase). These compounds are administered in regimens and at dosages known in the art.

Suk et al. (Stroke (2003) 34:1586-1592) discusses a link between being overweight or obese and strokes. Agents administered to treat strokes include anti-platelet agents (e.g., Aspirin, Clopidogrel, Dipyridamole, and Ticlopidine), anticoagulant agents (e.g., Heparin), and thrombolytic agents. Stein et al. (The American Journal of Medicine (2005) 18(9):978-980) discusses a link between being overweight or obese and venous thromboembolic disease. Agents administered to treat venous thromboembolic disease include anti-platelet agents, anticoagulant agents, and thrombolytic agents. Sztrymf et al. (Rev Pneumol Clin (2002) 58(2):104-10) discusses a link between being overweight or obese and pulmonary hypertension. Agents administered to treat pulmonary hypertension include inotropic agents, anticoagulant agents, diuretics, potassium (e.g., K-dur), vasodilators (e.g., Nifedipine and Diltiazem), Bosentan, Epoprostenol, and Sildenafil. Respiratory disorders and conditions such as obesity-hypoventilation syndrome, asthma, and obstructive sleep apnea, have been linked to being overweight or obese. Elamin (Chest (2004) 125:1972-1974) discusses a link between being overweight or obese and asthma. Agents administered to treat asthma include bronchodilators, anti-inflammatory agents, leukotriene blockers, and anti-Ige agents. Particular asthma agents include Zafirlukast, Flunisolide, Triamcinolone, Beclomethasone, Terbutaline, Fluticasone, Formoterol, Beclomethasone, Salmeterol, Theophylline, and Xopenex.

Kessler et al. (Eur Respir J (1996) 9:787-794) discusses a link between being overweight or obese and obstructive sleep apnea. Agents administered to treat sleep apnea include Modafinil and amphetamines.

Hepatic disorders and conditions, such as nonalcoholic fatty liver disease, have been linked to being overweight or obese. Tolman et al. (Ther Clin Risk Manag (2007) 6:1153-1163) discusses a link between being overweight or obese and nonalcoholic fatty liver disease. Agents administered to treat nonalcoholic fatty liver disease include antioxidants (e.g., Vitamins E and C), insulin sensitizers (Metformin, Pioglitazone, Rosiglitazone, and Betaine), hepatoprotectants, and lipid-lowering agents.

Skeletal disorders and conditions, such as, back pain and osteoarthritis of weight-bearing joints, have been linked to being overweight or obese. van Saase (J Rheumatol (1988) 15(7):1152-1158) discusses a link between being overweight or obese and osteoarthritis of weight-bearing joints. Agents administered to treat osteoarthritis of weight-bearing joints include Acetaminophen, non-steroidal anti-inflammatory agents (e.g., Ibuprofen, Etodolac, Oxaprozin, Naproxen, Diclofenac, and Nabumetone), COX-2 inhibitors (e.g., Celecoxib), steroids, supplements (e.g. glucosamine and chondroitin sulfate), and artificial joint fluid.

Metabolic disorders and conditions, for example, Prader-Willi Syndrome and polycystic ovary syndrome, have been linked to being overweight or obese. Agents administered to treat Prader-Willi Syndrome include human growth hormone (HGH), somatropin, and weight loss agents (e.g., Orlistat, Sibutramine, Methamphetamine, Ionamin, Phentermine, Bupropion, Diethylpropion, Phendimetrazine, Benzphetermine, and Topamax).

Hoeger (Obstetrics and Gynecology Clinics of North America (2001) 28(1):85-97) discusses a link between being overweight or obese and polycystic ovary syndrome. Agents administered to treat polycystic ovary syndrome include insulin-sensitizers, combinations of synthetic estrogen and progesterone, Spironolactone, Eflornithine, and Clomiphene. Reproductive disorders and conditions such as sexual dysfunction, erectile dysfunction, infertility, obstetric complications, and fetal abnormalities, have been linked to being overweight or obese. Larsen et al. (Int J Obes (Lond) (2007) 8:1189-1198) discusses a link between being overweight or obese and sexual dysfunction. Chung et al. (Eur Urol (1999) 36(1):68-70) discusses a link between being overweight or obese and erectile dysfunction. Agents administered to treat erectile dysfunction include phosphodiesterase inhibitors (e.g., Tadalafil, Sildenafil citrate, and Vardenafil), prostaglandin E analogs (e.g., Alprostadil), alkaloids (e.g., Yohimbine), and testosterone. Pasquali et al. (Hum Reprod (1997) 1:82-87) discusses a link between being overweight or obese and infertility. Agents administered to treat infertility include Clomiphene, Clomiphene citrate, Bromocriptine, Gonadotropin-releasing Hormone (GnRH), GnRH agonist, GnRH antagonist, Tamoxifen/nolvadex, gonadotropins, Human Chorionic Gonadotropin (HCG), Human Menopausal Gonadotropin (HmG), progesterone, recombinant follicle stimulating hormone (FSH), Urofollitropin, Heparin, Follitropin alfa, and Follitropin beta.

Weiss et al. (American Journal of Obstetrics and Gynecology (2004) 190(4):1091-1097) discusses a link between being overweight or obese and obstetric complications. Agents administered to treat obstetric complications include Bupivacaine hydrochloride, Dinoprostone PGE2, Meperidine HCl, Ferro-folic-500/iberet-folic-500, Meperidine, Methylergonovine maleate, Ropivacaine HCl, Nalbuphine HCl, Oxymorphone HCl, Oxytocin, Dinoprostone, Ritodrine, Scopolamine hydrobromide, Sufentanil citrate, and Oxytocic.

Psychiatric disorders and conditions, for example, weight-associated depression and anxiety, have been linked to being overweight or obese. Dixson et al. (Arch Intern Med (2003) 163:2058-2065) discusses a link between being overweight or obese and depression. Agents administered to treat depression include serotonin reuptake inhibitors (e.g., Fluoxetine, Escitalopram, Citalopram, Paroxetine, Sertraline, and Venlafaxine); tricyclic antidepressants (e.g., Amitriptyline, Amoxapine, Clomipramine, Desipramine, Dosulepin hydrochloride, Doxepin, Imipramine, Iprindole, Lofepramine, Nortriptyline, Opipramol, Protriptyline, and Trimipramine); monoamine oxidase inhibitors (e.g., Isocarboxazid, Moclobemide, Phenelzine, Tranylcypromine, Selegiline, Rasagiline, Nialamide, Iproniazid, Iproclozide, Toloxatone, Linezolid, Dienolide kavapyrone desmethoxyyangonin, and Dextroamphetamine); psychostimulants (e g, Amphetamine, Methamphetamine, Methylphenidate, and Arecoline); antipsychotics (e.g., Butyrophenones, Phenothiazines, Thioxanthenes, Clozapine, Olanzapine, Risperidone, Quetiapine, Ziprasidone, Amisulpride, Paliperidone, Symbyax, Tetrabenazine, and Cannabidiol); and mood stabilizers (e.g., Lithium carbonate, Valproic acid, Divalproex sodium, Sodium valproate, Lamotrigine, Carbamazepine, Gabapentin, Oxcarbazepine, and Topiramate).

Simon et al. (Archives of General Psychiatry (2006) 63(7):824-830) discusses a link between being overweight or obese and anxiety. Agents administered to treat anxiety include serotonin reuptake inhibitors, mood stabilizers, benzodiazepines (e.g., Alprazolam, Clonazepam, Diazepam, and Lorazepam), tricyclic antidepressants, monoamine oxidase inhibitors, and beta-blockers.

Another aspect of the present disclosure provides methods for facilitating and maintaining weight loss in a subject involving administering to the subject an amount of a disclosed compound effective to result in weight loss in the subject; and optionally administering a therapeutically effective amount of a different weight loss agent to maintain a reduced weight in the subject. Weight loss agents include serotonin and noradrenergic re-uptake inhibitors; noradrenergic re-uptake inhibitors; selective serotonin re-uptake inhibitors; and intestinal lipase inhibitors. Particular weight loss agents include orlistat, sibutramine, methamphetamine, ionamin, phentermine, bupropion, diethylpropion, phendimetrazine, benzphetermine, bromocriptine, lorcaserin, topiramate, or agents acting to modulate food intake by blocking ghrelin action, inhibiting diacylglycerol acyltransferase 1 (DGAT1) activity, inhibiting stearoyl CoA desaturase 1 (SCD1) activity, inhibiting neuropeptide Y receptor 1 function, activating neuropeptide Y receptor 2 or 4 function, or inhibiting activity of sodium-glucose cotransporters 1 or 2. These compounds are administered in regimens and at dosages known in the art.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as compounds of the present disclosure.

### Example A

### General Procedures

All reagents were purchased from commercial suppliers (Sigma-Aldrich, Alfa, Across etc.) and used without further purification unless otherwise stated. THF was continuously refluxed and freshly distilled from sodium and benzophenone under nitrogen, and dichloromethane was continuously refluxed and freshly distilled from CaH₂ under nitrogen.

Reactions were monitored by TLC on silica gel 60 HSGF254 percolated plates (0.15-0.2 mm SiO₂) and visualized using UV light (254 nm or 365 nm) and/or staining with phosphomolybdic acid ethanol solution (10 g in 100 mL ethanol) and subsequent heating or monitored by LCMS.

LCMS were performed on SHIMADZU LCMS-2010EV (Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min@ 10% B, 2.7 min gradient (10-95% B), then 0.8 min@95% B, Flow rate: 3 mL/min, temperature: 40° C.).

Preparative HPLC were performed either on Method A: SHIMADZU LC-8A (Column: YMC Pack ODS-A (150*30 mm, 10 μm)) or Method B: LC-6AD (Column: Shim=Pack PREP-ODS-H (250*20 mm, 10 μm)) with UV detection which were controlled by LC solution Chemstation software. H₂O (0.1% HCOOH) and MeOH (MeCN) as mobile phase at the indicated flow rate.

Analytical HPLC were performed on SHIMADZU LC-2010A (Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min@ 10% B, 2.7 min gradient (10-95% B), then 0.8 min@95% B, Flow rate: 3 mL/min, temperature: 40° C.).

Chiral HPLC were performed on SHIMADZU LC-2010A (Chiral column, mobile phase: Solvent A: hexane (or containing 0.1% diethylamine), Solvent B: Ethanol or Isopropanol; Flow rate: 0.8 mL/min, temperature: 30° C.).

¹H spectra were recorded on Bruker Avance II 400 MHz, Chemical shifts (δ) are reported in ppm relative to tetramethylsilane (δ=0.000 ppm), and the spectra were calibrated to the residual solvent signal of chloroform (δ=7.26), Dimethyl sulfoxide (δ=2.50), or methanol (δ=3.30). Data for ¹H-NMR spectra are reported as follows: chemical shift (multiplicity, number of hydrogens). Abbreviations are as follows: s (singlet), d (doublet), t (triplet), q (quartet), quint (quintet), m (multiple), br (broad).

### Abbreviations

Ac Acetyl
AcOH; HOAc acetic acid
aq. Aqueous
Bs Benzenesulfonyl
Cbz Benzyloxycarbonyl
CDI Carbonyldimidazole
DCM Dichloromethane
DEAD Diethyl azodicarboxylate
DIPEA Ethyldiisopropylamine
DMA Dimethyl acetamide
DMF Dimethyl formamide
EDCI/EDC 3-(ethyliminomethyleneamino)-N,N-dimethylpropan-1-amine
EtOH Ethanol
eq(s). equivalent(s)
EtOAc ethyl acetate
Et Ethyl
FA Formic acid
Et₃N Triethylamine
hr hour(s)
HATU (Dimethylamino)-N,N-dimethyl(3H-[1,2,3]triazolo[4,5-b]pyridin-3-yloxy)methaniminium hexafluorophosphate
HOBt 1-Hydroxybenzotriazole
HPLC High pressure liquid chromatography
LCMS; LC-MS liquid chromatography mass spectrometry
m-CPBA m-Chloroperoxybenzoic acid
MeOH Methanol
mg milligram(s)
min Minute(s)
mL; ml milliliter(s)
NCS N-Chlorosuccinimide
NMe N-methyl
NMO N-methylmorpholine-N-oxide
NMP N-methylpyrrolidinone
NMR Nuclear magnetic resonance
Pd(dppf)Cl₂ (1,1′-bis(diphenylphosphino) ferrocene) palladium (II) dichloride
PE Petroleum Ether
Ph Phenyl
PTSA p-Toluenesulfonic acid
r.t./RT Room temperature
S. Saturated
SEMCl 2-(Trimethylsilyl)ethoxymethyl chloride
TBAF Tetrabutylammonium fluoride
TEA Triethylamine
THF Tetrahydrofuran
THP Tetrahydropyran
TMSCN Trimethylsilyl cyanide
TMSOTf Trimethylsilyl Triflate
THF Tetrahydrofuran
TLC Thin layer chromatography
Ts Tosyl (4-methylbenzene-1-sulfonyl)

### PREPARATION OF INTERMEDIATES

### Intermediate 1

### (3R,4S,5S,6R)-5-methoxy-4-((2R,3R)-2-methyl-3-(3-methylbut-2-en-1-yl)oxiran-2-yl)-1-oxaspiro[2.5]octan-6-yl (4-nitrophenyl) carbonate

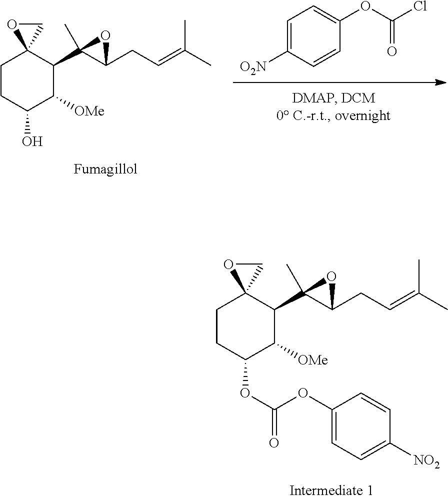 [SMILES: CO[C@@H]1[C@H](O)CC[C@]2(CO2)[C@H]1[C@@]1(C)O[C@@H]1CC=C(C)C ; O=C(Cl)Oc1ccc([N+](=O)[O-])cc1 ; CO[C@@H]1[C@H](OC(=O)Oc2ccc([N+](=O)[O-])cc2)CC[C@]2(CO2)[C@H]1[C@@]1(C)O[C@@H]1CC=C(C)C]

Fumagillol (40 g, 0.142 mol) and DMAP (34.6 g, 0.283 mol) were dissolved in anhydrous DCM (480 mL) with stirring at 0° C. A solution of p-nitrophenyl chloroformate (48.65 g, 0.241 mol) in DCM (250 mL) was added drop-wise to the mixture above for 1 hr, and the temperature was kept below 0° C. After addition was complete, the mixture was stirred at room temperature for 16 hrs. The mixture was diluted with DCM (500 mL), washed sequentially with a 10% aq. solution of citric acid, saturated aq. K₂CO₃ and brine. The organic layer was dried, concentrated and purified by silica gel chromatography (PE:EtOAc=20:1 to PE:EtOAc:DCM=5:1:1). The crude product was washed with PE/EtOAc (100 mL/30 mL) twice, hot EtOH (200 mL, ˜70° C.), dried under vacuum at r.t. to give (3R,4S,5S,6R)-5-methoxy-4-((2R,3R)-2-methyl-3-(3-methylbut-2-en-1-yl)oxiran-2-yl)-1-oxaspiro[2.5]octan-6-yl (4-nitrophenyl) carbonate as a white solid (47.9 g, 75.4% yield). LC-MS: m/z=448 [M+H]⁺. ¹H NMR (400 MHz, CDCl₃) δ 8.39-8.23 (m, 2H), 7.53-7.34 (m, 2H), 5.63 (d, J=2.8 Hz, 1H), 5.23 (t, J=7.4 Hz, 1H), 3.74 (dd, J=11.3, 2.6 Hz, 1H), 3.03 (d, J=4.2 Hz, 1H), 2.69-2.53 (m, 2H), 2.50-2.33 (m, 1H), 2.27-1.90 (m, 5H), 1.77 (s, 3H), 1.68 (s, 3H), 1.37-1.06 (m, 4H).

Procedures for the preparation of additional intermediates and compounds of the present disclosure are described in the following examples.

### Intermediate 2

### (S)-5-(1-amino-2-methylpropyl)-1,3,4-oxadiazol-2-amine

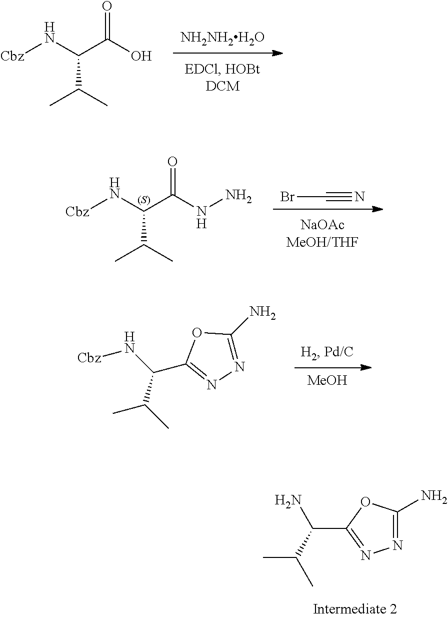

### Step 1: (S)-benzyl (1-hydrazinyl-3-methyl-1-oxobutan-2-yl)carbamate

To a solution of (S)-2-(benzyloxycarbonylamino)-3-methylbutanoic acid (3 g, 11.94 mmol) in dichloromethane (25 mL) was added 1-hydroxybenzotriazole (1.6 g, 11.9 mmol) followed by addition of 1-(3-dimethylaminopropyl)-3-ethylcarbodiimide, hydrochloride (2.4 g, 12.54 mmol) in portions at 0° C. The mixture was stirred at 0° C. for 30 min, and hydrazine hydrate (1.4 g, 23.9 mmol, 85% wt) was added drop-wise at 0° C. The reaction was then stirred at room temperature for 2 hrs. The mixture was then concentrated to dryness while keeping the temperature below 30° C. The residue was combined with ice-water, and the slurry was stirred for 10 min. The mixture was then filtered, and the filter cake was washed with water, followed by cold ethanol. The filter cake was then dried under vacuum to give crude product, which was re-crystallized from ethanol to give (S)-benzyl 1-hydrazinyl-3-methyl-1-oxobutan-2-ylcarbamate (1.6 g, 50.5% yield) as white solid. LC-MS: m/z=266 [M+H]⁺.

### Step 2: (S)-benzyl (1-(5-amino-1,3,4-oxadiazol-2-yl)-2-methylpropyl)carbamate

To a solution of (R)-benzyl 1-hydrazinyl-4-methyl-1-oxopentan-3-ylcarbamate (640 mg, 2.41 mmol) in methanol (12 mL) and THF (4 mL) was added sodium acetate (985 mg, 7.23 mmol), followed by addition of cyanogen bromide (383 mg, 3.62 mmol) in portions at 0° C. The reaction was stirred at room temperature for 4 hrs. The mixture was then concentrated under vacuum to give a residue, and the residue was dissolved in ethyl acetate, washed with water and brine, dried over Na₂SO₄, filtered and concentrated to give crude product. The crude product was purified by silica gel chromatography (petroleum ether:ethyl acetate=10:1 to 3:1) to give (S)-benzyl 1-(5-amino-1,3,4-oxadiazol-2-yl)-2-methylpropylcarbamate (610 mg, 87.2% yield) as a white solid. LC-MS: m/z=291 [M+H]⁺. ¹H NMR (400 MHz, DMSO) δ 7.91-7.93 (d, J=8.8 Hz, 1H), 7.31-7.35 (m, 5H), 6.97 (s, 2H), 5.03-5.08 (m, 2H), 4.39-4.44 (t, 1H), 2.03-2.12 (m, 1H), 0.93-0.94 (d, J=7.2 Hz, 3H), 0.83-0.85 (d, J=7.2 Hz, 3H), ee>98% (CHIRALPAK AD, 10% ethanol/hexane).

### Step 3: (S)-5-(1-amino-2-methylpropyl)-1,3,4-oxadiazol-2-amine

A solution of (S)-benzyl 1-(5-amino-1,3,4-oxadiazol-2-yl)-2-methylpropylcarbamate (350 mg, 1.2 mmol) in methanol (5 mL) was degassed three times under a N₂ atmosphere, and to this, Pd/C (35 mg, 10% wt) was added. The mixture was degassed again and stirred under a H₂ balloon at room temperature overnight. The reaction was filtered through Celite, and the filtrate was concentrated to give (S)-5-(1-amino-2-methylpropyl)-1,3,4-oxadiazol-2-amine (160 mg, 85.4% yield) as a light yellow oil. LC-MS: m/z=157 [M+H]⁺.

The following intermediates were prepared according to procedures similar to that described for Intermediate 2 by using the appropriate hydrazine.

| Inter- | | |
|---|---|---|
| mediate # | Structure | LC-MS |
| 3 | 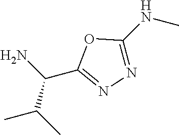 | 171 [M + H]⁺ |
| 4 | 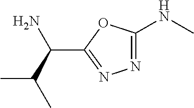 | 171 [M + H]⁺ |

### Intermediate 5

### (S)-1-(1H-imidazol-2-yl)-2-methylpropan-1-amine

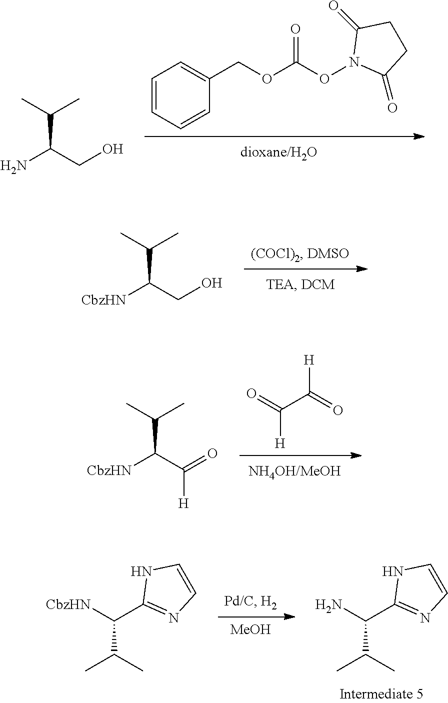

### Step 1: (S)-benzyl (1-hydroxy-3-methylbutan-2-yl)carbamate

(S)-2-amino-3-methylbutan-1-ol (5 g, 0.048 mol) was dissolved in dioxane/water (40 mL/10 mL) with constant stirring at room temperature. A solution of benzyl (2,5-dioxopyrrolidin-1-yl) carbonate (11 g, 0.044 mol) in dioxane/water (40 mL/10 mL) was added drop-wise to the mixture over 0.5 hr. The reaction was stirred at room temperature for 16 hrs. The mixture was then concentrated to a residue under vacuum while keeping the temperature below 40° C. The residue was diluted with ethyl acetate and washed sequentially with 5% aqueous sodium bicarbonate solution and 5% aqueous citric acid solution. The organic layer was dried with Na₂SO₄ and concentrated to give (S)-benzyl (1-hydroxy-3-methylbutan-2-yl)carbamate 10.1 g, 96.2% yield) as white solid. LC-MS: m/z=238 [M+H]+.

### Step 2: (S)-benzyl (3-methyl-1-oxobutan-2-yl)carbamate

To a mixture of oxalyl chloride (0.25 mL, 2.94 mmol) in anhydrous dichloromethane (5 mL) was added dimethyl sulfoxide (0.42 mL, 5.9 mmol) drop-wise at −78° C. After the addition was complete, the mixture was stirred −78° C. for 30 min (S)-benzyl (1-hydroxy-3-methylbutan-2-yl)carbamate (200 mg, 2.10 mmol) dissolved in anhydrous dichloromethane (20 mL) was added to the mixture drop-wise over 30 min, and the mixture was stirred for another 1 hr at −78° C. Triethylamine (0.85 mL, 6.10 mmol) was added, and the reaction was stirred at room temperature for 1 hr. TLC (petroleum ether:ethyl acetate=3:1) indicated that the starting material was completely consumed. The resulting mixture was washed with water (5 mL×2), saturated aqueous sodium bicarbonate (5 mL×2) and brine. The organic layer was dried over Na₂SO₄ and concentrated to give (S)-benzyl (3-methyl-1-oxobutan-2-yl)carbamate (400 mg, 80.1% yield) as a brown oil, which was used directly in the next reaction without purification.

### Step 3: (S)-benzyl (1-(1H-imidazol-2-yl)-2-methylpropyl)carbamate

To a mixture of (S)-benzyl (3-methyl-1-oxobutan-2-yl)carbamate (200 mg, 0.85 mmol) and oxalaldehyde (0.35 mL, 50% in water) in methanol (2 mL) was added ammonium hydroxide (0.37 mL, 35% wt) drop-wise while keeping the temperature below 10° C. The reaction was stirred at room temperature overnight. The reaction mixture was then poured into ice-water and filtered. The filter cake was washed with water to give (S)-benzyl (1-(1H-imidazol-2-yl)-2-methylpropyl)carbamate (125 mg, 53.6% yield) as a white solid. LC-MS: m/z=274 [M+H]⁺, ee>99% (CHIRALPAK AD, 15% ethanol/hexane).

### Step 4: (S)-1-(1H-imidazol-2-yl)-2-methylpropan-1-amine

A solution of (S)-benzyl (1-(1H-imidazol-2-yl)-2-methylpropyl)carbamate (200 mg, 0.729 mmol) in methanol (5 mL) was degassed three times under N₂, and Pd/C (20 mg, 10% wt) was then added in one portion under a N₂ atmosphere. The mixture was degassed again and stirred under a H₂ atmosphere at room temperature for 12 hrs. The reaction mixture was filtered through Celite, and the filtrate was concentrated under vacuum to give (S)-1-(1H-imidazol-2-yl)-2-methylpropan-1-amine (101 mg, 98.2% yield) as a colorless oil. LC-MS: m/z=140 [M+H]⁺.

### Intermediate 6

### (S)-2-methyl-1-(1-(((R)-1-methylpyrrolidin-2-yl)methyl)-1H-imidazol-2-yl)propan-1-amine

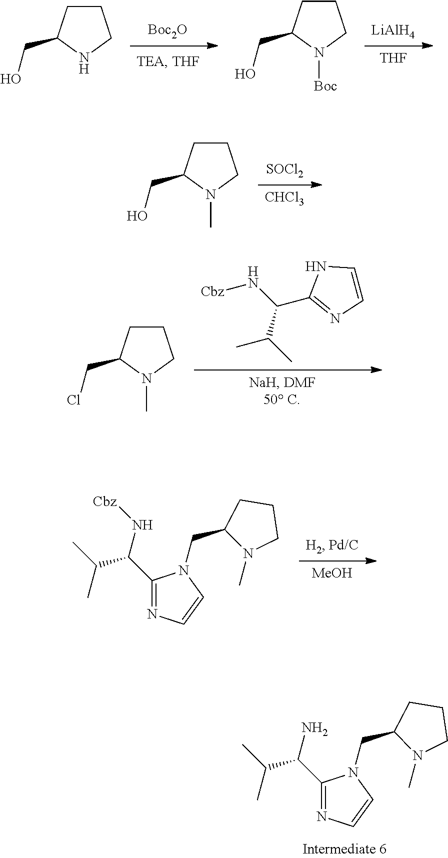

### Step 1: (R)-tert-butyl 2-(hydroxymethyl)pyrrolidine-1-carboxylate

To a solution of (R)-pyrrolidin-2-ylmethanol (5.0 g, 50.0 mmol) in THF (60 mL) was added triethylamine (7.6 g, 75.0 mmol), followed by drop-wise addition of a solution of di-tert-butyl dicarbonate (12.6 g, 60.0 mmol) in THF (20 mL). The reaction was stirred at room temperature overnight. The mixture was then poured into ice-water and extracted with ethyl acetate twice. The combined organic layers was washed with brine, dried over Na₂SO₄ and concentrated to give a residue, which was purified by silica gel chromatography (petroleum ether:ethyl acetate=10:1 to 3:1) to give (R)-tert-butyl 2-(hydroxymethyl)pyrrolidine-1-carboxylate (5.8 g, 58.2% yield) as a white solid. LC-MS (ESI) found: 146 [M+H−56]⁺.

### Step 2: (R)-(1-methylpyrrolidin-2-yl)methanol

To a solution of (R)-tert-butyl 2-(hydroxymethyl)pyrrolidine-1-carboxylate (5.8 g, 29.0 mmol) in THF (anhydrous, 60 mL) was added lithium aluminum hydride (3.28 g, 28.8 mmol) in portions at 0° C., and the reaction was stirred to 65° C. overnight. The mixture was quenched by aqueous sodium hydroxide solution (3.28 mL, 15% wt) and water (3.28 mL+9.84 mL). The slurry was filtered, and the filter cake was washed with ethyl acetate twice. The combined filtrates were dried over sodium sulfate and concentrated to give (R)-(1-methylpyrrolidin-2-yl)methanol (2.9 g, 67.1% yield) as a colorless oil. LC-MS (ESI) found: 116 [M+H−56]⁺.

### Step 3: (R)-2-(chloromethyl)-1-methylpyrrolidine

To a mixture of (R)-(1-methylpyrrolidin-2-yl)methanol (450 mg, 3.00 mmol) in chloroform (6 mL) was added thionyl chloride (1.5 mL) drop-wise at 0° C. The reaction was stirred at 65° C. overnight. The mixture was concentrated and washed with diethyl ether to give (R)-2-(chloromethyl)-1-methylpyrrolidine (410 mg, 80.35% yield) as yellow solid. LC-MS (ESI) found: 134 [M+H−56]⁺.

### Step 4: benzyl (S)-2-methyl-1-(1-(((R)-1-methylpyrrolidin-2-yl)methyl)-1H-imidazol-2-yl)propylcarbamate

To a mixture of (R)-2-(chloromethyl)-1-methylpyrrolidine (250 mg, 1.50 mmol) and (S)-benzyl 1-(1H-imidazol-2-yl)-2-methylpropylcarbamate (410 mg, 1.50 mmol) in DMF (5 mL) was added sodium hydride (120 mg, 3.00 mmol, 60% dispersion in mineral oil) in portions at 0° C. The reaction was stirred at room temperature for 16 hrs. The reaction mixture was then poured into ice-water and extracted with ethyl acetate twice. The combined organic layers was washed with brine, dried and concentrated to give crude product, which was purified by silica gel chromatography (dichloromethane:methanol=200:1 to 10:1) to give (S)-2-methyl-1-(1-(((R)-1-methylpyrrolidin-2-yl)methyl)-1H-imidazol-2-yl)propylcarbamate (250 mg, 45.4% yield) as a colorless oil. LC-MS (ESI) found: 371 [M+H−56]⁺.

### Step 5: (S)-2-methyl-1-(1-(((R)-1-methylpyrrolidin-2-yl)methyl)-1H-imidazol-2-yl)propan-1-amine

A solution of (S)-2-methyl-1-(1-(((R)-1-methylpyrrolidin-2-yl)methyl)-1H-imidazol-2-yl)propylcarbamate (220 mg, 0.59 mmol) in methanol (5 mL) was degassed under a N₂ atmosphere three times and Pd/C (30 mg, 10% wt) was added under a N₂ atmosphere. The mixture was degassed again and stirred under a H₂ balloon at room temperature overnight. The mixture was filtered, and the filter cake was washed with methanol twice. The combined filtrates were concentrated to dryness to give (S)-2-methyl-1-(1-(((R)-1-methylpyrrolidin-2-yl)methyl)-1H-imidazol-2-yl)propan-1-amine (190 mg, 95.1% yield) as a colorless oil. LC-MS (ESI) found: 237 [M+H]⁺.

The following intermediate was prepared according to a procedure similar to that described for Intermediate 6 from appropriate starting materials.

| Inter- | | |
|---|---|---|
| mediate # | Structure | LC-MS |
| 7 | 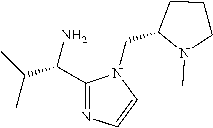 [SMILES: CC(C)[C@H](N)c1nccn1C[C@@H]1CCCN1C] | 237 [M + H]⁺ |

### Intermediate 8

### (R)-5-(1-amino-2-methylpropyl)-N-methyl-1,3,4-thiadiazol-2-amine

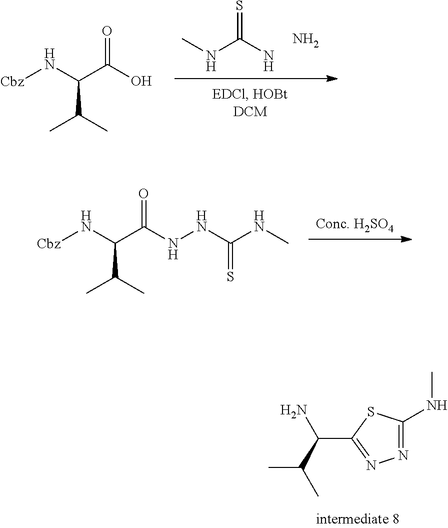

### Step 1: (R)-benzyl (3-methyl-1-(2-(methylcarbamothioyl)hydrazinyl)-1-oxobutan-2-yl)carbamate

To a solution of (R)-2-(benzyloxycarbonylamino)-3-methylbutanoic acid (3.0 g, 11.95 mmol) in dichloromethane (30 mL) was added HOBt (1.78 g, 13.15 mmol), followed by the addition of EDCI (2.52 g, 13.15 mmol) in portions at 0° C. The mixture was stirred at 0° C. for 10 min, and N-methylhydrazinecarbothioamide (1.63 g, 17.93 mmol) was added in one portion. The reaction was stirred at room temperature for 1 hr. The mixture was then diluted with dichloromethane, washed with saturated aqueous ammonium chloride solution, dried and concentrated to give (R)-benzyl (3-methyl-1-(2-(methylcarbamothioyl)hydrazinyl)-1-oxobutan-2-yl)carbamate (2.6 g, 64.4% yield) as a white solid. LC-MS (ESI) found: 339 [M+H]⁺, ee>99% (CHIRALPAK AD, 20% ethanol/hexane).

### Step 2: (R)-5-(1-amino-2-methylpropyl)-N-methyl-1,3,4-thiadiazol-2-amine

(R)-benzyl (3-methyl-1-(2-(methylcarbamothioyl)hydrazinyl)-1-oxobutan-2-yl)carbamate (1 g, 2.95 mmol) was added in portions to cooled concentrated sulfuric acid (5 mL) at 0° C. The reaction was stirred at 40° C. for 16 hrs. The reaction mixture was poured into ice-water, and the mixture was made basic with concentrated sodium hydroxide solution. The mixture was extracted with chloroform/isopropanol (3:1) three times. The combined organic layers were dried over anhydrous Na₂SO₄ and concentrated under reduced pressure to give (R)-5-(1-amino-2-methylpropyl)-N-methyl-1,3,4-thiadiazol-2-amine (240 mg, 44.3% yield) as a gray oil. LC-MS (ESI) found: 187 [M+H]⁺.

The following intermediate was prepared according to a procedure similar to that described for Intermediate 8.

| Inter- | | |
|---|---|---|
| mediate # | Structure | LC-MS |
| 9 | 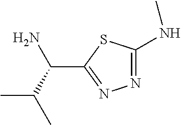 | 187 [M + H]⁺ |

### Intermediate 10

### (S)-2-methyl-1-(4H-1,2,4-triazol-3-yl)propan-1-amine

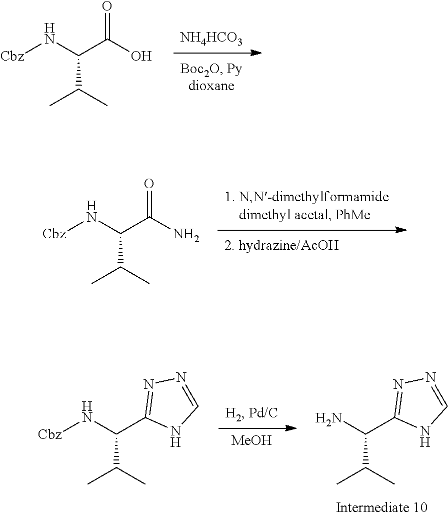

### Step 1: (S)-benzyl (1-amino-3-methyl-1-oxobutan-2-yl)carbamate

To a solution of (S)-2-(benzyloxycarbonylamino)-3-methylbutanoic acid (10.0 g, 39.84 mmol) in 1,4-dioxane (50 mL) was added di-tert-butyl dicarbonate (11.95 g, 51.79 mmol) in portions at 0° C. Then pyridine (2 mL, 25.76 mmol) was added drop-wise at 0° C., followed by ammonium bicarbonate (3.94 g, 50.43 mmol) in portions. The reaction was stirred at room temperature overnight. The mixture was poured into ice water and filtered. The filter cake was washed with water and dried under vacuum. The filter cake was then re-crystallized from dioxane/water (80 mL, 9:1, v/v) to give (S)-benzyl (1-amino-3-methyl-1-oxobutan-2-yl)carbamate (5.3 g, 53.15% yield) as a white solid. LC-MS (ESI) found: 251 [M+H]⁺, ee>99%, (CHIRALPAK AS-H, 15% ethanol/hexane)

### Step 2: (S)-benzyl (2-methyl-1-(4H-1,2,4-triazol-3-yl)propyl)carbamate

To a solution of (S)-benzyl 1-amino-3-methyl-1-oxobutan-2-ylcarbamate (1.00 g, 4.00 mmol) in toluene (anhydrous, 20 mL) was added N,N′-dimethylformamide dimethyl acetal (476.1 mg, 4.00 mmol) The mixture was stirred at 100° C. for 30 min. The mixture was then concentrated to dryness. The residue was dissolved in acetic acid (20 mL), and hydrazine (256.1 mg, 8.00 mmol) was added to the mixture. The reaction was stirred at 90° C. for 1 hr. The mixture was poured into ice-water (100 mL), and the resulting mixture was stirred for 10 min. The slurry was filtered, and the filter cake was washed with water and ethyl acetate/methanol (1:3, v/v) and dried under vacuum to give (S)-benzyl (2-methyl-1-(4H-1,2,4-triazol-3-yl)propyl)carbamate (510 mg, 46.4% yield) as a white solid. LC-MS (ESI) found: 275 [M+H]⁺; ee=98.65% (CHIRALPAK AD, 15% ethanol/hexane); ¹H NMR (400 MHz, DMSO) δ 8.19 (s, 1H), 7.63 (s, 1H), 7.45-7.24 (m, 5H), 5.17-4.90 (m, 2H), 4.51 (t, J=8.2 Hz, 1H), 2.12 (dt, J=13.5, 6.8 Hz, 1H), 0.98-0.64 (m, 6H).

### Step 3: (S)-2-methyl-1-(4H-1,2,4-triazol-3-yl)propan-1-amine

A solution of (S)-benzyl (2-methyl-1-(4H-1,2,4-triazol-3-yl)propyl)carbamate (800 mg, 2.92 mmol) in methanol (20 mL) was degassed three times under a N₂ atmosphere, and Pd/C (50 mg, 10% wt) was added. The resulting mixture was degassed again and stirred under a H₂ balloon at room temperature overnight. The mixture was filtered, and the filter cake was washed with methanol twice. The combined filtrates were concentrated to dryness to give (S)-2-methyl-1-(4H-1,2,4-triazol-3-yl)propan-1-amine (390 mg, 95.3% yield) as a white solid, which was directly used in the next reaction without purification. LC-MS (ESI) found: 141 [M+H]⁺.

### Intermediate 11

### (S)-3-(1-amino-2-methylpropyl)-1,2,4-oxadiazol-5(4H)-one, hydrobromide

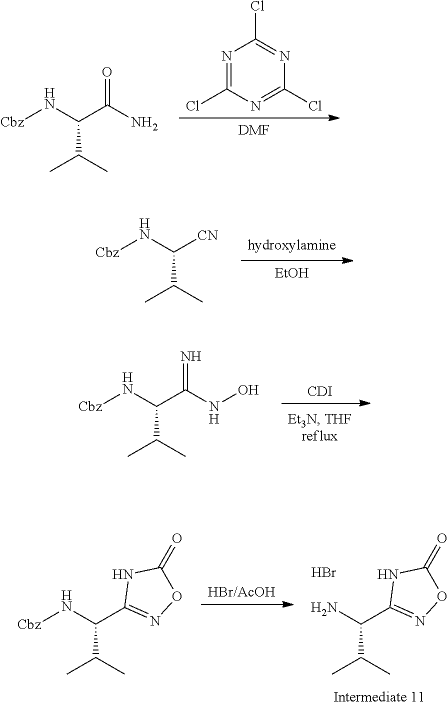

### Step 1: (S)-benzyl (1-cyano-2-methylpropyl)carbamate

To a solution of (S)-benzyl 1-amino-3-methyl-1-oxobutan-2-ylcarbamate (1.7 g, 6.79 mmol) in DMF (anhydrous, 30 mL) was added 2,4,6-trichloro-1,3,5-triazine (1.75 g, 9.51 mmol) at 0° C. The reaction was stirred at room temperature for 2 hrs. The reaction was poured into water (300 mL), and the mixture was extracted with ethyl acetate (50 mL*3). The combined organic layers were washed with water and brine, dried over Na₂SO₄, filtered and concentrated to give a residue, which was purified by silica gel chromatography (petroleum ether:ethyl acetate=10:1) to give (S)-benzyl (1-cyano-2-methylpropyl)carbamate (1.5 g, 95.2% yield) as a colorless oil. LC-MS (ESI) found: 233 [M+H]⁺, ee=95.97% (CHIRALPAK AS-H, 5% ethanol/hexane).

### Step 2: (S)-benzyl (1-(hydroxyamino)-1-imino-3-methylbutan-2-yl)carbamate

To a solution of (S)-benzyl (1-cyano-2-methylpropyl)carbamate (1.5 g, 6.48 mmol) in ethanol (40 mL) was added hydroxylamine (1.7 g, 25.8 mmol, 50% wt in ethanol). The reaction was stirred at room temperature overnight. The mixture was concentrated to remove ethanol and the residue was dissolved in ethyl acetate. The mixture was washed with brine, dried over Na₂SO₄, filtered and concentrated to give crude product, which was purified by silica gel chromatography (petroleum ether:ethyl acetate=3:1 to 1:1) to give (S)-benzyl (1-(hydroxyamino)-1-imino-3-methylbutan-2-yl)carbamate (1.5 g, 87.7% yield) as a white solid. LC-MS (ESI) found: 266 [M+H]⁺, ee=97.89% (CHIRALPAK AS-H, 5% ethanol/hexane).

### Step 3: (S)-benzyl (2-methyl-1-(5-oxo-4,5-dihydro-1,2,4-oxadiazol-3-yl)propyl)carbamate

To a solution of (S)-benzyl (1-(hydroxyamino)-1-imino-3-methylbutan-2-yl)carbamate (1.1 g, 4.15 mmol) in THF (anhydrous, 100 mL) was added triethylamine (0.21 g, 2.07 mmol) under a N₂ atmosphere. The mixture was heated to reflux, and a solution of 1,1′-carbonyldiimidazole (1.01 g, 6.22 mmol) in THF (10 mL) was added drop-wise. The reaction was stirred at reflux overnight. Water (50 mL) was added to the reaction, and the mixture was extracted with ethyl acetate twice. The combined organic layers were washed with brine, dried over Na₂SO₄, filtered, and concentrated to give crude product, which was purified by silica gel chromatography (dichloromethane:methanol=500:3) to give (S)-benzyl (2-methyl-1-(5-oxo-4,5-dihydro-1,2,4-oxadiazol-3-yl)propyl)carbamate (500 mg, 58.3% yield) as a white solid. LC-MS (ESI) found: 292 [M+H]⁺, ee=98.25% (CHIRALPAK AS-H, 20% ethanol/hexane).

### Step 4: (S)-3-(1-amino-2-methylpropyl)-1,2,4-oxadiazol-5(4H)-one, hydrobromide

A mixture of (S)-benzyl (2-methyl-1-(5-oxo-4,5-dihydro-1,2,4-oxadiazol-3-yl)propyl)carbamate (700 mg, 2.40 mmol) in hydrogen bromide-acetic acid solution (5 mL, 20% wt) was stirred at room temperature for 2 hrs. The mixture was concentrated to dryness, washed with diethyl ether twice, and dried under vacuum to give (S)-3-(1-amino-2-methylpropyl)-1,2,4-oxadiazol-5(4H)-one, hydrobromide (590 mg, 87.1% yield) as a grey solid. LC-MS (ESI) found: 158 [M+H]⁺. ¹H NMR (400 MHz, DMSO) δ 8.77 (s, 3H), 4.27-4.26 (d, J=4.00 Hz, 2H), 2.26-2.21 (m, 1H), 0.99-0.97 (d, J=8.00 Hz, 1H).

The following intermediate was prepared according to a procedure similar to that described for Intermediate 11 by using the corresponding enantiomer.

| Intermediate | | |
|---|---|---|
| # | Structure | LC-MS |
| 12 | 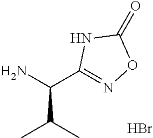 | 158 [M + H]⁺ |

### Intermediate 13

### (R)-2-methyl-1-(1-(((S)-1-methylpyrrolidin-3-yl)methyl)-1H-imidazol-4-yl)propan-1-amine, hydrochloride

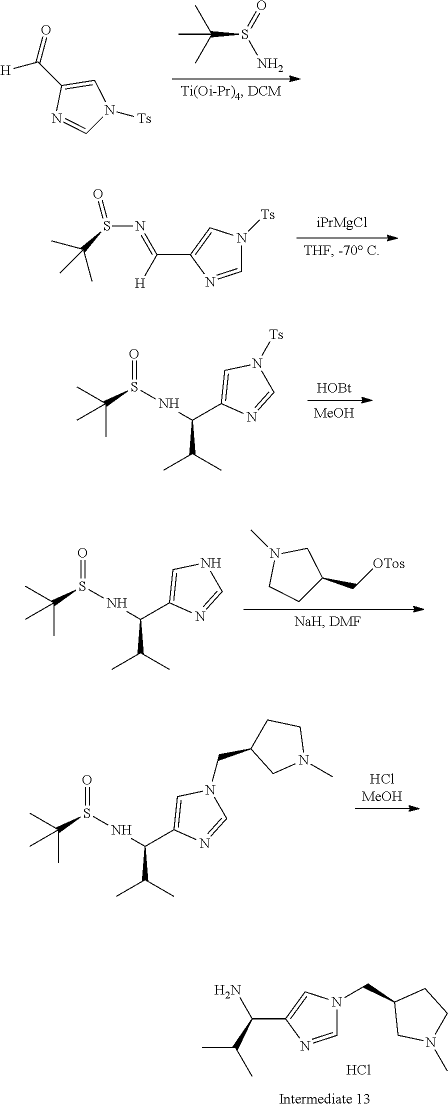

### Step 1: (R,E)-2-methyl-N-((1-tosyl-1H-imidazol-4-yl)methylene)propane-2-sulfinamide

To a mixture of 1-tosyl-1H-imidazole-4-carbaldehyde (3.0 g, 11.99 mmol) and (R)-2-methylpropane-2-sulfinamide (1.74 g, 14.38 mmol) in tetrahydrofuran (30 mL) was added titanium tetraisopropanolate (5.11 g, 17.98 mmol) drop-wise at 0° C. After addition was complete, the mixture was stirred at 45° C. for 3 hours. The reaction was quenched by addition of water and extracted with ethyl acetate (50 mL×2). The organic layers were washed with water and brine, dried over anhydrous Na₂SO₄ and concentrated. The residue was purified by silica gel chromatography (petroleum ether:ethyl acetate=5:1 to 2:1) to give the titled product (3.0 g, 71.0% yield) as a white solid. LC-MS: m/z=354 [M+H]⁺.

### Step 2: (R)-2-methyl-N—((R)-2-methyl-1-(1-tosyl-1H-imidazol-4-yl)propyl)propane-2-sulfinamide

To a solution of (R,E)-2-methyl-N-((1-tosyl-1H-imidazol-4-yl)methylene)propane-2-sulfinamide (3.0 g, 8.5 mmol) in tetrahydrofuran (10 mL) was added isopropylmagnesium chloride (8.5 mL, 17.0 mmol) drop-wise at −78° C. After addition was complete, the mixture was stirred at −78° C. for 1 h. The reaction was quenched by addition of saturated aqueous NH₄Cl solution and extracted with ethyl acetate (50 mL×2). The organic layers were dried over anhydrous Na₂SO₄, filtered and concentrated. The residue was purified by silica gel chromatography (petroleum ether:ethyl acetate=4:1 to 1:1) to give the titled product (2.6 g, 77.5% yield) as a colorless oil. LC-MS: m/z=398 [M+H]⁺.

### Step 3: (R)—N—((R)-1-(1H-imidazol-4-yl)-2-methylpropyl)-2-methylpropane-2-sulfinamide

To a solution of (R)-2-methyl-N—((R)-2-methyl-1-(1-tosyl-1H-imidazol-4-yl)propyl)propane-2-sulfinamide (2.6 g, 6.55 mmol) in methanol (30 mL) was added HOBt (1.77 g, 13.1 mmol) at 0° C. After addition was complete, the mixture was stirred at 25° C. overnight. The mixture was concentrated and partitioned with ethyl acetate (50 mL) and aq. HCl (40 mL, 1.0 M). The aqueous layer was adjusted to pH 8 with sat. aq. Na₂CO₃ and extracted with dichloromethane (150 mL×3). The organic layers were dried over anhydrous Na₂SO₄, filtered and concentrated under reduced pressure to give the titled product (1.3 g, 80.1% yield) as a white solid. LC-MS: m/z=244 [M+H]⁺.

### Step 4: (R)-2-methyl-N—((R)-2-methyl-1-(1-(((S)-1-methylpyrrolidin-3-yl)methyl)-1H-imidazol-4-yl)propyl)propane-2-sulfinamide

To a solution of (R)—N—((R)-1-(1H-imidazol-4-yl)-2-methylpropyl)-2-methylpropane-2-sulfinamide (500 mg, 2.05 mmol) in DMF (8 mL) was added NaH (147.6 mg, 6.15 mmol) in portions at 0° C. The mixture was stirred at 0° C. for 30 min. (S)-(1-methylpyrrolidin-3-yl)methyl 4-methylbenzenesulfonate was added to the mixture in portions. After addition was complete, the mixture was stirred at 50° C. for 2 hrs. The reaction was quenched by addition of ice water and extracted with ethyl acetate (100 mL×2). The organic layers were dried over anhydrous Na₂SO₄, filtered and concentrated. The residue was purified by silica gel chromatography (DCM:MeOH=50:1 to 10:1) to give the titled compound (310 mg, 44.3% yield) as a brown oil. LC-MS: m/z=341 [M+H]⁺.

### Step 5: (R)-2-methyl-1-(1-(((S)-1-methylpyrrolidin-3-yl)methyl)-1H-imidazol-4-yl)propan-1-amine, hydrochloride

To a solution of (R)-2-methyl-N—((R)-2-methyl-1-(1-(((S)-1-methylpyrrolidin-3-yl)methyl)-1H-imidazol-4-yl)propyl)propane-2-sulfinamide (310 mg, 0.91 mmol) in MeOH (2 mL) was added HCl methanol solution (3 mL, 2 M). After stirring at room temperature for 1 hr, the mixture was concentrated to give (R)-2-methyl-1-(1-(((S)-1-methylpyrrolidin-3-yl)methyl)-1H-imidazol-4-yl)propan-1-amine, hydrochloride (215 mg) as a white solid, which was used directly in the next step without purification. LC-MS: m/z=237 [M+H]⁺.

The following intermediates were prepared according to procedures similar to that described for Intermediate 13 by using the corresponding tosylates.

| Intermediate | | |
|---|---|---|
| # | Structure | LC-MS |
| 14 | 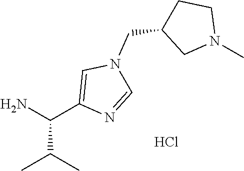 [SMILES: CC(C)[C@H](N)c1cn(C[C@@H]2CCN(C)C2)cn1 ; Cl] | 237 [M + H]⁺ |
| 15 | 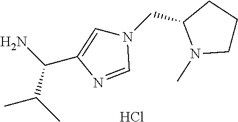 [SMILES: CC(C)[C@H](N)c1cn(C[C@@H]2CCCN2C)cn1 ; Cl] | 237 [M + H]⁺ |
| 16 | 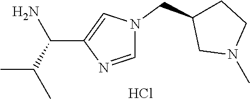 [SMILES: CC(C)[C@H](N)c1cn(C[C@H]2CCN(C)C2)cn1 ; Cl] | 237 [M + H]⁺ |
| 17 | 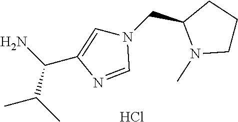 [SMILES: CC(C)[C@H](N)c1cn(C[C@H]2CCCN2C)cn1 ; Cl] | 237 [M + H]⁺ |
| 18 | 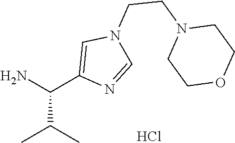 [SMILES: CC(C)[C@H](N)c1cn(CCN2CCOCC2)cn1 ; Cl] | 253 [M + H]⁺ |

### Intermediate 19

### (S)-1-(1-(((S)-1-(2,2-difluoroethyl)pyrrolidin-3-yl)methyl)-1H-imidazol-4-yl)-2-methylpropan-1-amine

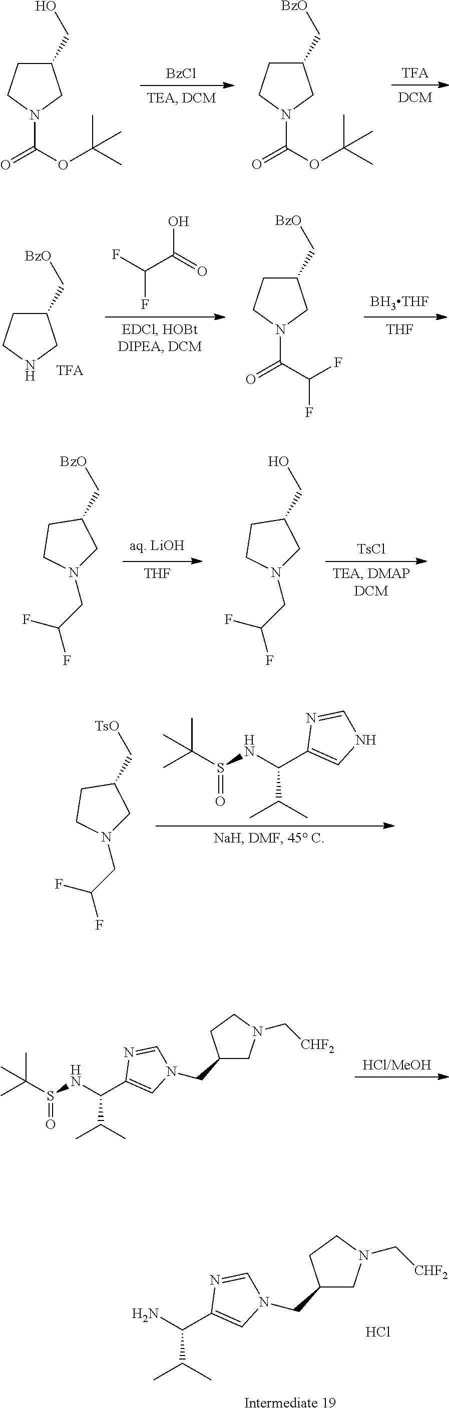

### Step 1: (S)-tert-butyl 3-(benzoyloxymethyl)pyrrolidine-1-carboxylate hydrochloride

To a solution of (S)-tert-butyl 3-(hydroxymethyl)pyrrolidine-1-carboxylate (1.5 g, 7.45 mmol) in DCM (40 mL) was added TEA (2.2 mL, 15 mmol) dropwise at 0° C., followed by dropwise addition of benzoyl chloride (1.26 g, 8.94 mmol). After addition was completed, the reaction was stirred at room temperature overnight. The reaction mixture was then washed with aqueous HCl (1 M, 50 mL). The water layer was then extracted with EtOAc (30 mL×3), and the combined EtOAc extracts were dried over Na₂SO₄, filtered and concentrated to give (S)-tert-butyl 3-(benzoyloxymethyl)pyrrolidine-1-carboxylate (1.9 g, 83.48% yield) as a colorless oil. LC-MS m/z: 250 [M+H−56]⁺.

### Step 2: (S)-pyrrolidin-3-ylmethyl benzoate

To a mixture of (S)-tert-butyl 3-(benzoyloxymethyl)pyrrolidine-1-carboxylate (1.9 g, 6.22 mmol) in DCM (10 mL) was added TFA (5.0 mL) drop-wise at 0° C. The reaction was stirred room temperature for 1 hr. The mixture was concentrated under reduced pressure to give (S)-pyrrolidin-3-ylmethyl benzoate trifluoroacetic acid salt (1.2 g, 93.96% yield) as a white solid, which was directly used to next step without purification. LC-MS m/z: 206 [M+H]⁺.

### Step 3: (S)-(1-(2,2-difluoroacetyl)pyrrolidin-3-yl)methyl benzoate

To a mixture of 2,2-difluoroacetic acid (1.15 g, 12 mmol) in DCM (40 mL) was sequentially added EDCI (3.07 g, 16 mmol) and HOBt (1.95 g, 14.4 mmol), and then DIPEA (6.19 g, 48 mmol) was added dropwise. After the mixture was stirred for 10 min, (S)-pyrrolidin-3-ylmethyl benzoate trifluoroacetic acid salt (1.2 g, 6 mmol) was added. The reaction was stirred at room temperature overnight. The reaction was diluted with DCM (50 mL×2) and washed with water (50 mL), brine (50 mL), dried over Na₂SO₄ and concentrated. The residue was purified by silica gel chromatography (DCM:MeOH=100:1 to 50:1 to give (S)-(1-(2,2-difluoroacetyl)pyrrolidin-3-yl)methyl benzoate (950 mg, 57.36% yield) as a colorless oil. LC-MS m/z: 284 [M+H]⁺.

### Step 4: (S)-(1-(2,2-difluoroethyl)pyrrolidin-3-yl)methyl benzoate

To a solution of (S)-(1-(2,2-difluoroacetyl)pyrrolidin-3-yl)methyl benzoate (900 mg, 3.18 mmol) in anhydrous THF (50 mL) was added a solution of borane-tetrahydrofuran complex (18.5 mL, 18.5 mmol, 1M in THF) dropwise at −10° C. over 10 min. The reaction was stirred at room temperature overnight. To the reaction mixture was then added MeOH (20 mL) dropwise at 0° C. After addition was completed, the mixture was stirred at room temperature for 30 min. The mixture was concentrated under vacuum to give a yellow reside, which was dissolved in EtOH/H₂O (9 mL/1 mL) and stirred at reflux overnight. The mixture was concentrated under vacuum to give a residue, which was purified by silica gel column chromatography (DCM:MeOH=100:1 to 40:1) to give (S)-(1-(2,2-difluoroethyl)pyrrolidin-3-yl)methyl benzoate (760 mg, 84.15% yield) as a colorless oil. LC-MS m/z: 235 [M+H−56]⁺.

### Step 5: (S)-(1-(2,2-difluoroethyl)pyrrolidin-3-yl)methanol

To a solution of (S)-(1-(2,2-difluoroethyl)pyrrolidin-3-yl)methyl benzoate (760 mg, 2.82 mmol) in THF (30 mL) was added a solution of aq. LiOH (6.0 mL, 12.0 mmol, 2M in water) dropwise at 0° C. over 10 min. The reaction was stirred at room temperature overnight. The reaction was diluted with DCM (50 mL×2) and washed with water (50 mL), brine (50 mL), dried over Na₂SO₄. The organic layer was concentrated under vacuum to give a residue, which was purified by silica gel column chromatography (DCM:MeOH=100:1 to 40:1) to give (S)-(1-(2,2-difluoroethyl)pyrrolidin-3-yl)methanol (410 mg, 87.95% yield) as a colorless oil. LC-MS m/z: 166 [M+H]⁺.

### Step 6: (S)-(1-(2,2-difluoroethyl)pyrrolidin-3-yl)methyl 4-methylbenzenesulfonate

To a solution of (S)-(1-(2,2-difluoroethyl)pyrrolidin-3-yl)methanol (410 mg, 2.48 mmol) in DCM (10 mL) was added TEA (0.76 g, 7.5 mmol) and DMAP (30 mg, 0.25 mmol) at 0° C. After the mixture was stirred at 0° C. for 0.5 hr, 4-methylbenzene-1-sulfonyl chloride (710 mg, 3.72 mmol) was added in portions, and the reaction was stirred at room temperature overnight. The mixture was poured into the ice water and extracted with EtOAc (50 mL×2). The organic layer was dried over Na₂SO₄, filtered and concentrated to give a residue, which was purified by silica gel column chromatography (petroleum ether:EtOAc=10:1 to 2:1) to give (S)-(1-(2,2-difluoroethyl)pyrrolidin-3-yl)methyl 4-methylbenzenesulfonate (610 mg, 76.95% yield) as a white solid. LC-MS m/z: 320 [M+H]⁺.

### Step 7: (S)—N—((S)-1-(1-(((S)-1-(2,2-difluoroethyl)pyrrolidin-3-yl)methyl)-1H-imidazol-4-yl)-2-methylpropyl)-2-methylpropane-2-sulfinamide

To a solution of (S)—N—((S)-1-(1H-imidazol-4-yl)-2-methylpropyl)-2-methylpropane-2-sulfinamide (600 mg, 2.30 mmol) in DMF (10 mL) was added NaH (279 mg, 6.99 mmol) in portions at 0° C. under N₂ atmosphere. The mixture was stirred at room temperature for 30 min and a solution of (S)-(1-(2,2-difluoroethyl)pyrrolidin-3-yl)methyl 4-methylbenzenesulfonate (410 mg, 1.91 mmol) in DMF (5 mL) was added dropwise. The reaction was stirred at 50° C. overnight. The reaction was quenched with ice-water and extracted with EtOAc. The combined organic layers were washed with water and brine, dried and concentrated to give a yellow residue. The residue was purified by silica gel column chromatography (DCM:MeOH=20:1 to 5:1) to give (S)—N—((S)-1-(1-(((S)-1-(2,2-difluoroethyl)pyrrolidin-3-yl)methyl)-1H-imidazol-4-yl)-2-methylpropyl)-2-methylpropane-2-sulfinamide (410 mg, 55.04% yield) as a yellow oil. LC-MS m/z: 391[M+H]⁺.

### Step 8: (S)-1-(1-(((S)-1-(2,2-difluoroethyl)pyrrolidin-3-yl)methyl)-1H-imidazol-4-yl)-2-methylpropan-1-amine

To a solution of (S)—N—((S)-1-(1-(((S)-1-(2,2-difluoroethyl)pyrrolidin-3-yl)methyl)-1H-imidazol-4-yl)-2-methylpropyl)-2-methylpropane-2-sulfinamide (410 mg, 1.05 mmol) in MeOH (25 mL) was added a HCl/MeOH solution (1 M, 3.0 mL, 3.0 mmol). The reaction was stirred at room temperature for 5 hrs. The reaction mixture was concentrated to dryness and washed with diethyl ether. Evaporation of the solvent gave (S)-2-methyl-1-(1-(((S)-1-methylpyrrolidin-2-yl)methyl)-1H-imidazol-4-yl)propan-1-amine hydrochloride (280 mg, 93.24% yield) as a yellow solid, which was directly used to next reaction without purification. LC-MS m/z: 287 [M+H]⁺.

The following intermediate was prepared according to procedures similar to that described for Intermediate 19 by using the corresponding starting materials.

| Inter- | | |
|---|---|---|
| medi- | | |
| ate | | |
| # | Structure | LC-MS |
| 20 | 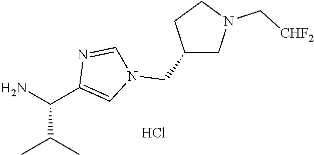 [SMILES: CC(C)[C@H](N)c1cn(C[C@@H]2CCN(CC(F)F)C2)cn1 ; Cl] | 287 [M + H]⁺ |

### Intermediate 21

### (S)-2-(1-amino-2-methylpropyl)pyridin-4-ol, hydrochloride

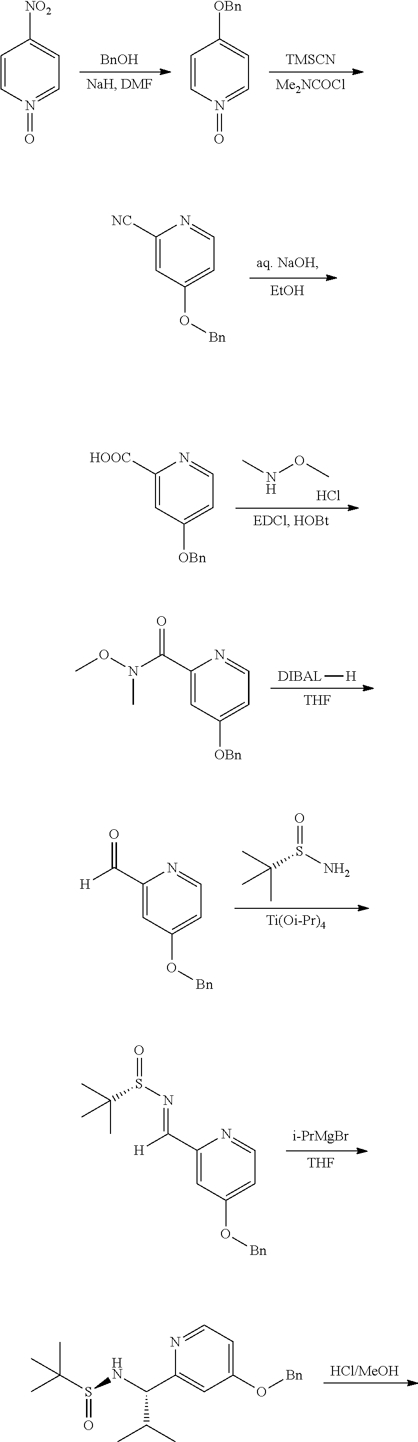

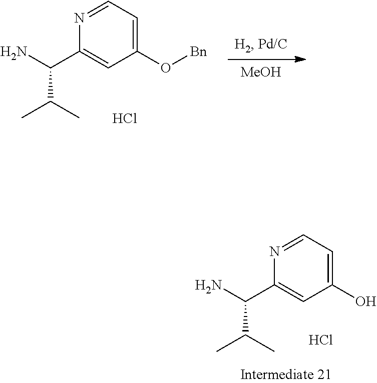

### Step 1: 4-(benzyloxy)pyridine 1-oxide

To a solution of benzyl alcohol (4.04 mL, 29.3 mmol) in DMF (25 mL) was added sodium hydride (2.86 g, 71.4 mmol, 60% dispersion in mineral oil) in portions at 0° C. After addition was complete, the mixture was stirred at room temperature for 1 hr. 4-Nitropyridine N-oxide (5 g, 35.7 mmol) was then added in small portions, and the reaction was stirred at 100° C. overnight. The reaction mixture was diluted with brine and extracted with chloroform (50 mL*5). The combined organic layers was dried over Na₂SO₄, filtered, and concentrated under reduced pressure to give 4-(benzyloxy)pyridine 1-oxide (5.5 g, 78.1% yield) as a brown solid, which was directly used in the next reaction without purification. LC-MS (ESI) found: 201 [M+H]⁺.

### Step 2: 4-(benzyloxy)picolinonitrile

To a solution of 4-(benzyloxy)pyridine 1-oxide (2.68 g, 13.3 mmol) in dichloromethane (15 mL) was added trimethylsilyl cyanide (2.0 mL, 15.7 mmol), followed by drop-wise addition of a solution of dimethylcarbamic chloride (1.5 mL, 15,733 mmol) in dichloromethane (5 mL) at 0° C. The reaction was stirred at room temperature overnight. Aqueous sodium bicarbonate solution (15 mL, 10% wt) was added, and the mixture was stirred for 20 min. The resulting mixture was extracted with dichloromethane, and the combined organic layers was washed with brine, dried over Na₂SO₄ and concentrated under reduced pressure to give 4-(benzyloxy)picolinonitrile (2.43 g, 86.8% yield) as a white solid. LC-MS (ESI) found: 211 [M+H]⁺.

### Step 3: 4-(benzyloxy)picolinic acid

To a solution of 4-(benzyloxy)picolinonitrile (2.2 g, 10.46 mmol) in ethanol (15 mL) was added 10% aqueous sodium hydroxide solution (15 mL). The reaction was stirred at 90° C. for 1 hr. The resulting mixture was cooled to room temperature and acidified by adding 2M hydrochloric acid to pH-3. The mixture was filtered and the filter cake was washed with water and dried under vacuum to give 4-(benzyloxy)picolinic acid (1.5 g, 62.5% yield) as white solid. LC-MS (ESI) found: 230 [M+H]⁺.

### Step 4: 4-(benzyloxy)-N-methoxy-N-methylpicolinamide

To a solution of 4-(benzyloxy)picolinic acid (1.5 g, 6.55 mmol) in DMF (20 mL) was added 1-hydroxybenzotriazole (916 mg, 7.37 mmol) and triethylamine (3.65 mL, 26.2 mmol), followed by addition of 1-(3-dimethylaminopropyl)-3-ethylcarbodiimide, hydrochloride ((1.89 g, 9.83 mmol)) in portions at 0° C. The resulting mixture was stirred at room temperature for 15 min, and N,O-dimethylhydroxylamine, hydrochloride (965 mg, 9.83 mmol) was added. The reaction was stirred at room temperature overnight. The reaction mixture was partitioned between water and ethyl acetate. The combined organic layers was washed with brine, dried over anhydrous Na₂SO₄ and concentrated under reduced pressure to give a residue, which was purified by silica gel chromatography (petroleum ether:ethyl acetate=5:1) to give 4-(benzyloxy)-N-methoxy-N-methylpicolinamide (1.54 g, 86.4% yield) as a yellow solid. LC-MS (ESI) found: 273 [M+H]⁺.

### Step 5: 4-(benzyloxy)picolinaldehyde

To a solution of 4-(benzyloxy)-N-methoxy-N-methylpicolinamide (1.15 g, 4.23 mmol) in THF (anhydrous, 15 mL) was added diisobutylaluminum hydride (8.46 mL, 12.69 mmol, 1 M in toluene) drop-wise at −78° C. under a N₂ atmosphere. The resulting mixture was stirred at −78° C. for 1.5 hrs. The reaction was quenched by addition of acetone (7 mL), and the solvent was removed under reduced pressure. The residue was diluted with ethyl acetate and washed with brine, dried over anhydrous Na₂SO₄ and concentrated under reduced pressure to give 4-(benzyloxy)picolinaldehyde (810 mg, 89.8% yield) as a colorless oil, which was directly used in next step without purification. LC-MS (ESI) found: 214 [M+H]⁺.

### Step 6: (S,E)-N-((4-(benzyloxy)pyridin-2-yl)methylene)-2-methylpropane-2-sulfinamide

To a solution of 4-(benzyloxy)picolinaldehyde (4.8 g, 22.54 mmol) and (S)-2-methylpropane-2-sulfinamide (3.28 g, 27.04 mmol) in THF (anhydrous, 10 mL) was added titanium tetraisopropanolate (10 mL, 33.82 mmol) drop-wise at 0° C. The reaction was stirred at 45° C. overnight and was then diluted with ethyl acetate and water. The mixture was stirred for 30 min and filtered through a Celite pad. The filtrate was extracted with ethyl acetate twice. The combined organic layers was washed with brine, dried over anhydrous Na₂SO₄ and concentrated under reduced pressure to give a residue which was purified by silica gel chromatography (dichloromethane:methanol=50:1) to give (S,E)-N-((4-(benzyloxy)pyridin-2-yl)methylene)-2-methylpropane-2-sulfinamide (800 mg, 11.2% yield) as a yellow oil. LC-MS (ESI) found: 317 [M+H]⁺.

### Step 7: (S)—N—((S)-1-(4-(benzyloxy)pyridin-2-yl)-2-methylpropyl)-2-methylpropane-2-sulfinamide

To a solution of (S,E)-N-((4-(benzyloxy)pyridin-2-yl)methylene)-2-methylpropane-2-sulfinamide (800 mg, 2.532 mmol) in THF (anhydrous, 15 mL) was added iso-propylmagnesium bromide (2.5 mL, 5.06 mmol, 2 M in THF) drop-wise at −78° C. under a N₂ atmosphere. The reaction was stirred at −78° C. for 2 hrs. The reaction was then quenched by drop-wise addition of saturated aqueous ammonium chloride and extracted with ethyl acetate. The combined organic layers was washed with brine, dried over anhydrous Na₂SO₄ and concentrated under reduced pressure to give a residue, which was purified by silica gel chromatography (dichloromethane:methanol=100:1) to give (S)—N—((S)-1-(4-(benzyloxy)pyridin-2-yl)-2-methylpropyl)-2-methylpropane-2-sulfinamide (280 mg, 30.6% yield) as yellow oil. LC-MS (ESI) found: 361 [M+H]⁺.

### Step 8: (S)-1-(4-(benzyloxy)pyridin-2-yl)-2-methylpropan-1-amine, hydrochloride

A solution of (S)—N—((S)-1-(4-(benzyloxy)pyridin-2-yl)-2-methylpropyl)-2-methylpropane-2-sulfinamide (280 mg, 0.77 mmol) in an HCl-methanol solution (10 mL, 1M) was stirred at room temperature overnight. The mixture was then concentrated under reduced pressure to give (S)-1-(4-(benzyloxy)pyridin-2-yl)-2-methylpropan-1-amine, hydrochloride (210 mg, crude) as a colorless oil, which was directly used in the next reaction without purification. LC-MS (ESI) found: 257 [M+H]⁺.

### Step 9: (S)-2-(1-amino-2-methylpropyl)pyridin-4-ol, hydrochloride

A solution of (S)-1-(4-(benzyloxy)pyridin-2-yl)-2-methylpropan-1-amine, hydrochloride (210 mg, crude, 0.77 mmol) in MeOH (5 mL) was degassed three times under a N₂ atmosphere, then Pd/C (25 mg, 10% wt) was added in one portion. After addition was complete, the mixture was degassed again and stirred under a H₂ balloon at room temperature overnight. The resulting mixture was filtered through a pad of Celite and the filtrate was concentrated under vacuum to give (S)-1-(4-(benzyloxy)pyridin-2-yl)-2-methylpropan-1-amine (120 mg, 93.7% yield) as a colorless oil. LC-MS (ESI) found: 167 [M+H]⁺.

### Intermediate 22

### (S)-2-methyl-1-(1H-1,2,3-triazol-4-yl)propan-1-amine, hydrobromide

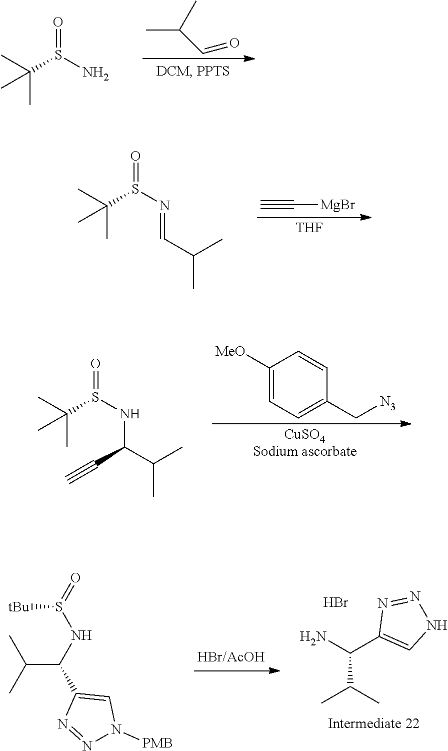

### Step 1: (S,E)-2-methyl-N-(2-methylpropylidene)propane-2-sulfinamide

To a solution of (S)-2-methylpropane-2-sulfinamide (2.40 g, 20.0 mmol) in dichloromethane (40 mL) was added 2-methyl-propionaldehyde (2.88 g, 40.0 mmol), followed by pyridinium toluene-4-sulphonate (250 mg, 1.00 mmol) and magnesium sulfate (12 g, 100.0 mmol). The reaction was stirred at room temperature overnight. TLC (petroleum ether/ethyl acetate=1:1) showed the reaction was complete. The mixture was filtered, and the filtrate was concentrated to give a residue, which was purified by silica gel column (dichloromethane:methanol=200:1 to 100:1) to give (S,E)-2-methyl-N-(2-methylpropylidene)propane-2-sulfinamide (2.9 g, 82.9% yield) as a colorless oil. LC-MS (ESI) found: 176 [M+H]⁺.

### Step 2: (S)-2-methyl-N—((S)-4-methylpent-1-yn-3-yl)propane-2-sulfinamide

To a solution of (S,E)-2-methyl-N-(2-methylpropylidene)propane-2-sulfinamide (1.05 g, 6.00 mmol) in THF (anhydrous, 10 mL) was added ethynylmagnesium bromide (24 mL, 12.0 mmol, 0.5 M in THF). The mixture was stirred at room temperature overnight, and TLC (petroleum ether/ethyl acetate=3:1) showed the reaction was complete. The mixture was quenched with aqueous ammonium chloride and extracted with ethyl acetate. The organic layer was washed with brine, dried over sodium sulfate and concentrated to give a residue, which was purified by silica gel chromatography (petroleum ether:ethyl acetate=10:1 to 4:1) to give (S)-2-methyl-N—((S)-4-methylpent-1-yn-3-yl)propane-2-sulfinamide (350 mg, 30.1% yield) as a colorless oil. LC-MS (ESI) found: 202 [M+H]⁺.

### Step 3: (S)—N—((S)-1-(1-(4-methoxybenzyl)-1H-1,2,3-triazol-4-yl)-2-methylpropyl)-2-methylpropane-2-sulfinamide

1-(Azidomethyl)-4-methoxybenzene (1.03 g, 6.0 mmol) was added to a solution of (S)-2-methyl-N—((S)-4-methylpent-1-yn-3-yl)propane-2-sulfinamide (1.05 g, 5.00 mmol) in tert-butanol/water (25 mL/2 mL), followed by cupric sulfate hydrate (25 mg, 0.1 mmol, 5H₂O) and sodium ascorbate (134 mg, 0.67 mmol). The reaction was stirred at room temperature overnight. The reaction mixture was diluted with ethyl acetate and washed with brine. The organic layer was dried over Na₂SO₄ and concentrated to give crude product, which was purified by silica gel column (petroleum ether:ethyl acetate=10:1 to 1:1) and prep. HPLC (Method B, H₂O (0.1% FA)/CH₃CN to give (S)—N—((S)-1-(1-(4-methoxybenzyl)-1H-1,2,3-triazol-4-yl)-2-methylpropyl)-2-methylpropane-2-sulfinamide (920 mg, 50.5% yield) as white solid. LC-MS (ESI) found: 365 [M+H]⁺.

### Step 4: (S)-2-methyl-1-(1H-1,2,3-triazol-4-yl)propan-1-amine, hydrobromide

A solution of (S)—N—((S)-1-(1-(4-methoxybenzyl)-1H-1,2,3-triazol-4-yl)-2-methylpropyl)-2-methylpropane-2-sulfinamide (720 mg, 2.03 mmol) in an aqueous hydrogen bromide solution (5 mL, 37% wt) was stirred at 100° C. overnight. The mixture was concentrated to dryness and washed with diethyl ether to give (S)-2-methyl-1-(1H-1,2,3-triazol-4-yl)propan-1-amine, hydrobromide (350 mg) as a brown solid, which was directly used in the next reaction without purification. LC-MS (ESI) found: 141 [M+H]⁺.

### Intermediate 23

### (S)-2-methyl-1-(1-(((S)-1-methylpyrrolidin-3-yl)methyl)-1H-pyrazol-4-yl)propan-1-amine, hydrochloride

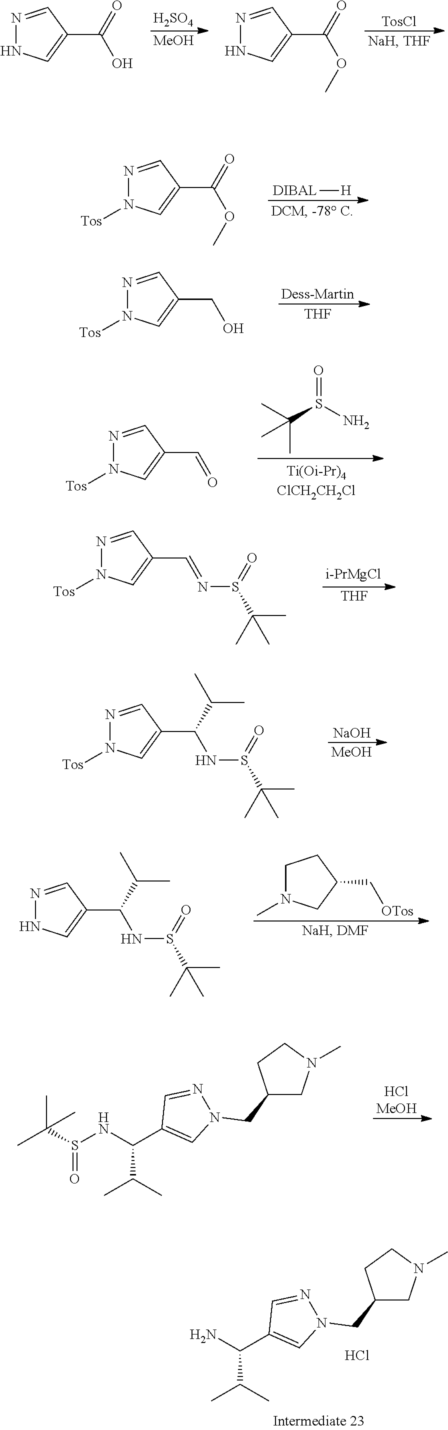

### Step 1: methyl 1H-pyrazole-4-carboxylate

To a mixture of 1H-pyrazole-4-carboxylic acid (5.00 g, 44.6 mmol) in dry CH₃OH (100 mL) was added drop-wise H₂SO₄ (874 mg, 8.92 mmol) at 0° C. The mixture was stirred at reflux overnight. A solution of NaOH (3.83 g, 95.7 mmol, 10 N) was added drop-wise to the reaction mixture. The mixture was poured into ice-water and diluted with ethyl acetate. The organic layer was washed with saturated aqueous lithium chloride solution and brine successively, dried over anhydrous Na₂SO₄ and concentrated. The residue was purified by silica gel chromatography (petroleum ether:ethyl acetate=10:1) to give methyl 1H-pyrazole-4-carboxylate (4.50 g) as a white solid. LC-MS (ESI) found: 127 [M+H]⁺.

### Step 2: methyl 1-tosyl-1H-pyrazole-4-carboxylate

To a solution of methyl 1H-pyrazole-4-carboxylate (2.81 g, 22.3 mmol) in THF (50 mL) was added sodium hydride (1.10 g, 27.5 mmol, 60% dispersion in mineral moil) in portions at 0° C. The mixture was stirred at 0° C. for 30 min. 4-Methylbenzene-1-sulfonyl chloride (4.66 g, 24.5 mmol) was added in portions, and the reaction was stirred at room temperature overnight. The mixture was poured into ice water and extracted with ethyl acetate (100 mL×2). The organic layers were dried over anhydrous Na₂SO₄, filtered and concentrated. The residue was purified by silica gel chromatography (petroleum ether:ethyl acetate=8:1 to 3:1) to give methyl 1-tosyl-1H-pyrazole-4-carboxylate (4.10 g) as a white solid. LC-MS (ESI) found: 281 [M+H]⁺.

### Step 3: (1-tosyl-1H-pyrazol-4-yl)methanol

To a −78° C. solution of 1-(toluene-4-sulfonyl)-1H-pyrazole-4-carboxylic acid ethyl ester (4.10 g, 14.6 mmol) in dichloromethane was added DIBAL-H (52.4 mL, 52.4 mmol, 1M solution in DCM) drop-wise. After stirred at −78° C. for 2 hours, the reaction was warmed to room temperature and stirred overnight. The reaction was quenched by addition of saturated aqueous NaHCO₃ at 0° C. The suspension was filtered through the diatomaceous earth and washed with DCM. The organic layer was washed with brine, dried over anhydrous Na₂SO₄ and concentrated. The residue was purified by silica gel chromatography (petroleum ether:ethyl acetate=10:1 to 2:1) to give (1-tosyl-1H-pyrazol-4-yl)methanol (2.62 g) as a white solid. LC-MS (ESI) found: 253 [M+H]⁺.

### Step 4: 1-tosyl-1H-pyrazole-4-carbaldehyde

To a solution of [1-(toluene-4-sulfonyl)-1H-pyrazol-4-yl]-methanol (2.35 g, 9.3 mmol) in THF (30 mL) was added Dess-Martin periodinane (6.78 g, 15.9 mmol). The reaction was stirred at room temperature overnight. The mixture was poured into ice water and extracted with ethyl acetate. The combined organic layers were washed with brine, dried over anhydrous Na₂SO₄ and concentrated. The residue was purified by silica gel chromatography (petroleum ether:ethyl acetate=15:1 to 4:1) to give 1-tosyl-1H-pyrazole-4-carbaldehydel (2.20 g) as a white solid. LC-MS (ESI) found: 251 [M+H]⁺.

### Step 5: (R,E)-2-methyl-N-((1-tosyl-1H-pyrazol-4-yl)methylene)propane-2-sulfinamide

To a solution of 1-(toluene-4-sulfonyl)-1H-pyrazole-4-carbaldehyde (2.20 g, 8.7 mmol) in dichloroethane was added (R)-2-methylpropane-2-sulfinamide (1.95 g, 16.8 mmol) and titanium (IV) isopropoxide (5.60 g, 19.6 mmol) at 0° C. After the reaction was stirred at reflux for 12 hrs, it was cooled and concentrated in vacuo. The residue was partitioned between ice water and ethyl acetate, and the aqueous phase was extracted with ethyl acetate. The combined organic layers were washed with brine, dried over anhydrous Na₂SO₄ and concentrated. The residue was purified by silica gel chromatography (petroleum ether:ethyl acetate=10:1 to 4:1) to give (R,E)-2-methyl-N-((1-tosyl-1H-pyrazol-4-yl)methylene)propane-2-sulfinamide (2.90 g) as a white solid. LC-MS (ESI) found: 354 [M+H]⁺.

### Step 6: (R)-2-methyl-N—((S)-2-methyl-1-(1-tosyl-1H-pyrazol-4-yl)propyl)propane-2-sulfinamide

To a solution of (R,E)-2-methyl-N-((1-tosyl-1H-pyrazol-4-yl)methylene)propane-2-sulfinamide (2.60 g 7.3 mmol) in THF (40 mL) was added isopropylmagnesium chloride (8.1 mL, 16.2 mmol, 2 M in THF) drop-wise at −70° C. The reaction was then stirred at −70° C. for 1 hr. The reaction mixture was quenched with saturated aqueous ammonium chloride and extracted with ethyl acetate (80 mL×2). The organic layers were dried over anhydrous Na₂SO₄, filtered and concentrated. The residue was purified by silica gel chromatography (petroleum ether:ethyl acetate=10:1 to 3:1) to give (R)-2-methyl-N—((S)-2-methyl-1-(1-tosyl-1H-pyrazol-4-yl)propyl)propane-2-sulfinamide (1.32 g) as a white solid. LC-MS (ESI) found: 398 [M+H]⁺. ¹H NMR (400 MHz, DMSO-d₆) δ 8.00 (s, 1H), 7.82 (d, J=8.8 Hz, 2H), 7.60 (s, 1H), 7.26 (d, J=8.8 Hz, 2H), 5.14-5.17 (m, 1H), 3.15 (d, J=6.4 Hz, 1H), 235 (s, 3H), 2.05-2.09 (m, 1H), 1.11 (s, 9H), 0.77-0.83 (m, 6H).

### Step 7: (R)-2-methyl-N—((S)-2-methyl-1-(1H-pyrazol-4-yl)propyl)propane-2-sulfinamide

To a solution of (R)-2-methyl-N—((S)-2-methyl-1-(1-tosyl-1H-pyrazol-4-yl)propyl)propane-2-sulfinamide (1.1 g, 2.77 mmol) in methanol (10 mL) was added aqueous NaOH (6.1 mL, 0.5 N) at 0° C. The reaction was stirred at 25° C. overnight. The mixture was then concentrated and poured into ice water and extracted with ethyl acetate. The combined organic layers were washed with brine, dried over anhydrous Na₂SO₄ and concentrated. The residue was purified by silica gel chromatography (dichloromethane:methanol=50:1 to 10:1) to give (R)-2-methyl-N—((S)-2-methyl-1-(1H-pyrazol-4-yl)propyl)propane-2-sulfinamide (520 mg) as a white solid. LC-MS (ESI) found: 244 [M+H]⁺, ¹H NMR (400 MHz, DMSO-d₆) 7.36 (s, 2H), 4.12-4.15 (m, 1H), 3.46 (d, J=9.2 Hz, 1H), 2.04-2.09 (m, 1H), 1.17 (s, 9H), 0.80-0.85 (m, 6H), 235 (s, 3H), 2.05-2.09 (m, 1H), 1.11 (s, 9H), 0.77-0.83 (m, 6H).

### Step 8: (R)-2-methyl-N—((S)-2-methyl-1-(1-(((S)-1-methylpyrrolidin-3-yl)methyl)-1H-pyrazol-4-yl)propyl)propane-2-sulfinamide

To a solution of (R)-2-methyl-N—((S)-2-methyl-1-(1H-pyrazol-4-yl)propyl)propane-2-sulfinamide (486 mg, 2.2 mmol) and (S)-(1-methylpyrrolidin-3-yl)methyl 4-methylbenzenesulfonate (605 mg, 2.2 mmol) in DMF (8 mL) was added sodium hydride (405 mg, 6.1 mmol, 60% dispersion in mineral oil) in portions at 0° C. After stirring at 50° C. overnight, the reaction was quenched with ice water and extracted with ethyl acetate (60 mL×4). The combined organic layers was dried over anhydrous Na₂SO₄, filtered and concentrated. The residue was purified by silica gel chromatography (dichloromethane:methanol=100:1 to 8:1) to give (R)-2-methyl-N—((S)-2-methyl-1-(1-(((S)-1-methylpyrrolidin-3-yl)methyl)-1H-pyrazol-4-yl)propyl)propane-2-sulfinamide (410 mg) as a brown oil. LC-MS (ESI) found: 341 [M+H]⁺.

### Step 9: (S)-2-methyl-1-(1-(((S)-1-methylpyrrolidin-2-yl)methyl)-1H-imidazol-4-yl)propan-1-amine, hydrochloride

To a solution of ((R)-2-methyl-N—((S)-2-methyl-1-(1-(((S)-1-methylpyrrolidin-3-yl)methyl)-1H-pyrazol-4-yl)propyl)propane-2-sulfinamide (410 mg, 2.6 mmol) in methanol (10 mL) was added HCl methanol solution (2.1 mL, 2 N). The reaction was stirred at 50° C. for 2 hrs. The reaction mixture was concentrated to dryness to give (S)-2-methyl-1-(1-(((S)-1-methylpyrrolidin-3-yl)methyl)-1H-pyrazol-4-yl)propan-1-amine, hydrochloride (610 mg) as a yellow solid, which was directly used in the next step without purification. LC-MS (ESI) found: 271 [M+H]⁺.

The following intermediates were prepared according to procedures similar to that described for Intermediate 23 by using the corresponding tosylate.

| Intermediate | | |
|---|---|---|
| # | Structure | LC-MS |
| 24 | 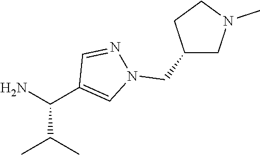 | 341 [M + H]⁺ |

### Intermediate 25

### (S)-2-methyl-1-(2-methyl-1-(((S)-1-methylpyrrolidin-3-yl)methyl)-1H-imidazol-4-yl)propan-1-amine

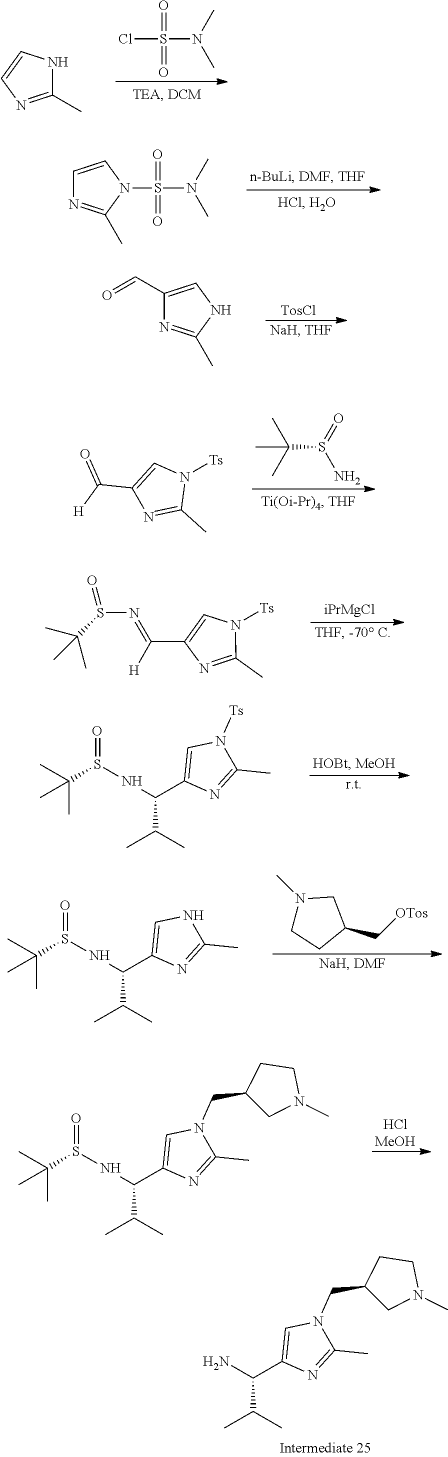

### Step 1: N,N,2-trimethyl-1H-imidazole-1-sulfonamide

To a solution of 2-methyl-1H-imidazole (4.0 g, 48.7 mmol) and triethylamine (5.5 g 55 mmol) in dichloroethane (50 mL) was added dimethylsulfamoyl chloride (14.0 g, 97.44 mmol) drop-wise at 0° C. The reaction was stirred at room temperature overnight. The solid was removed by filtration and washed with dichloroethane. The combined filtrates were washed with saturated Na₂CO₃ solution, dried over Na₂SO₄, filtered and concentrated to give a residue, which was purified by silica gel chromatography (PE:EtOAc=100:1 to 1:1) to give N,N,2-trimethyl-1H-imidazole-1-sulfonamide (8.0 g, 86.72% yield) as a colorless oil. LC-MS: m/z=190 [M+H]⁺.

### Step 2: 2-methyl-1H-imidazole-4-carbaldehyde

To a solution of N,N,2-trimethyl-1H-imidazole-1-sulfonamide (8.0 g, 42.28 mmol) in dry THF (100 mL) was added n-BuLi (50.7 mL, 50.7 mmol 1 M in THF) dropwise at −78° C. After the mixture was stirred at −78° C. for 30 min, DMF (19.37 g, 265.3 mmol) was added dropwise. The reaction mixture was stirred at −78° C. for 1 h and then allowed to warm to room temperature and stirred for 2 hrs. The reaction was quenched by addition of water and adjusted to pH 1 with concentrated HCl solution and the resulting mixture was stirred for 2 hrs. Then the pH value was adjusted to 8 with aqueous NaHCO₃ solution. The mixture was extracted with ethyl acetate (80 mL×3). The organic layers were dried over Na₂SO₄, filtered and concentrated to give a residue, which was purified by silica gel chromatography (PE:EtOAc=10:1 to 1:1) to give 2-methyl-1H-imidazole-4-carbaldehyde (2.6 g, 28.31% yield) as a yellow solid. LC-MS: 111 [M+H]⁺.

### Step 3: 2-methyl-1-tosyl-1H-imidazole-4-carbaldehyde

To a solution of 2-methyl-1H-imidazole-4-carbaldehyde (2.2 g, 20.0 mmol) in THF (50 mL) was added NaH (1.2 g, 30 mmol, 60% dispersion in mineral oil) in portions at 0° C. After the mixture was stirred at 0° C. for 0.5 hr, 4-methylbenzene-1-sulfonyl chloride (4.57 g, 24.0 mmol) was added in portions, and the reaction was stirred at room temperature overnight. The mixture was poured into the ice water and extracted with ethyl acetate (100 mL×2). The organic layers were dried over Na₂SO₄, filtered and concentrated to give a residue, which was purified by silica gel chromatography (PE:EtOAc=10:1 to 1:1) to give 1-tosyl-1H-imidazole-4-carbaldehyde (3.2 g, 60.6% yield) as a white solid. LC-MS: m/z=265 [M+H]⁺.

### Step 4: (S,E)-2-methyl-N-((2-methyl-1-tosyl-1H-imidazol-4-yl)methylene)propane-2-sulfinamide

To a mixture of 1-tosyl-1H-imidazole-4-carbaldehyde (3.0 g, 11.35 mmol) and (S)-2-methylpropane-2-sulfinamide (1.65 g, 13.62 mmol) in THF (60 mL) was added titanium tetraisopropanolate (5.04 mL, 17.03 mmol) drop-wise at 0° C. The reaction was stirred at 45° C. overnight. The reaction mixture was quenched with water and filtered. The filter pad was washed with ethyl acetate, and the filtrate was extracted with ethyl acetate (100 mL×3). The organic layer was dried over Na₂SO₄, filtered and concentrated. The concentrate was purified by silica gel chromatography (PE:EtOAc=10:1 to 2:1) to give (S,E)-2-methyl-N-((2-methyl-1-tosyl-1H-imidazol-4-yl)methylene)propane-2-sulfinamide (4.0 g, 95.9% yield) as a white solid. LC-MS: m/z=368 [M+H]⁺.

### Step 5: (S)-2-methyl-N—((S)-2-methyl-1-(2-methyl-1-tosyl-1H-imidazol-4-yl)propyl)propane-2-sulfinamide

To a solution of (S,E)-2-methyl-N-((2-methyl-1-tosyl-1H-imidazol-4-yl)methylene)propane-2-sulfinamide (4.0 g, 10.88 mmol) in THF (50 mL) was added iso-propylmagnesium chloride (10.88 mL, 21.76 mmol 2 M in THF) drop-wise at −70° C. The reaction was then stirred at −70° C. for 1 hr. The reaction mixture was quenched with saturated aqueous NH₄Cl and extracted with ethyl acetate (200 mL×2). The organic layers were dried over Na₂SO₄, filtered and concentrated. The concentrate was purified by silica gel chromatography (petroleum ether:ethyl acetate=10:1 to 4:3) to give (S)-2-methyl-N—((S)-2-methyl-1-(2-methyl-1-tosyl-1H-imidazol-4-yl)propyl)propane-2-sulfinamide (3.1 g, 69.3% yield) as a white solid. LC-MS: m/z=412 [M+H]⁺.

### Step 6: (S)—N—((S)-1-(1H-imidazol-4-yl)-2-methylpropyl)-2-methylpropane-2-sulfinamide

To a solution of (S)-2-methyl-N—((S)-2-methyl-1-(2-methyl-1-tosyl-1H-imidazol-4-yl)propyl)propane-2-sulfinamide (1.0 g, 2.43 mmol) in methanol (20 mL) was added 1-hydroxybenzotriazole (0.66 g, 4.87 mmol) at 0° C. After addition, the reaction was stirred at 25° C. overnight. The reaction mixture was then partitioned between EtOAc and aqueous HCl solution (30 mL, 1.0 M). The aqueous HCl phase was made basic with saturated Na₂CO₃ solution and exacted with dichloromethane (50 mL×3). The combined organic layers was dried over Na₂SO₄, filtered and concentrated to give (S)—N—((S)-1-(1H-imidazol-4-yl)-2-methylpropyl)-2-methylpropane-2-sulfinamide (600 mg, 96.0% yield) as a white solid. LC-MS: m/z=258 [M+H]⁺.

### Step 7: (S)-2-methyl-N—((S)-2-methyl-1-(2-methyl-1-(((S)-1-methylpyrrolidin-3-yl)methyl)-1H-imidazol-4-yl)propyl)propane-2-sulfinamide

To a solution of (S)—N—((S)-1-(1H-imidazol-4-yl)-2-methylpropyl)-2-methylpropane-2-sulfinamide (600 mg, 2.33 mmol) in DMF (10 mL) was added NaH (279 mg, 6.99 mmol) in portions at 0° C. under N₂ atmosphere. The mixture was stirred at room temperature for 30 min and a solution of (S)-(1-methylpyrrolidin-3-yl)methyl 4-methylbenzenesulfonate (753.5 mg, 2.80 mmol) in DMF (5 mL) was added drop-wise. The reaction was stirred at 50° C. overnight. The reaction was quenched with ice-water and extracted with EtOAc (10 mL×2). The combined organic layers were washed with water and brine, dried and concentrated. The residue was purified by silica gel chromatography (dichloromethane:methanol=20:1 to 5:1) to give (S)-2-methyl-N—((S)-2-methyl-1-(2-methyl-1-(((S)-1-methylpyrrolidin-3-yl)methyl)-1H-imidazol-4-yl)propyl)propane-2-sulfinamide (450 mg, 54.45% yield) as a yellow oil. LC-MS: m/z=355 [M+H]⁺.

### Step 8: (S)-2-methyl-1-(2-methyl-1-(((S)-1-methylpyrrolidin-3-yl)methyl)-1H-imidazol-4-yl)propan-1-amine

To a solution of (S)-2-methyl-N—((S)-2-methyl-1-(2-methyl-1-(((S)-1-methylpyrrolidin-3-yl)methyl)-1H-imidazol-4-yl)propyl)propane-2-sulfinamide (450 mg, 1.27 mmol) in methanol (10 mL) was added HCl-methanol solution (3 mL, 2M) drop-wise at 0° C. The reaction was stirred at room temperature for 3 hrs. The resulting mixture was concentrated under vacuum to give a residue, which was azeotroped with toluene (10 mL×3) to give (S)-2-methyl-1-(2-methyl-1-(((S)-1-methylpyrrolidin-3-yl)methyl)-1H-imidazol-4-yl)propan-1-amine (210 mg, 90% yield) as a brown oil, which was directly used to the next reaction without purification. LC-MS: m/z=251 [M+H]⁺.

### Intermediate 26

### (S)-1-(6-(3,3-difluoroazetidin-1-yl)pyridin-3-yl)-2-methylpropan-1-amine

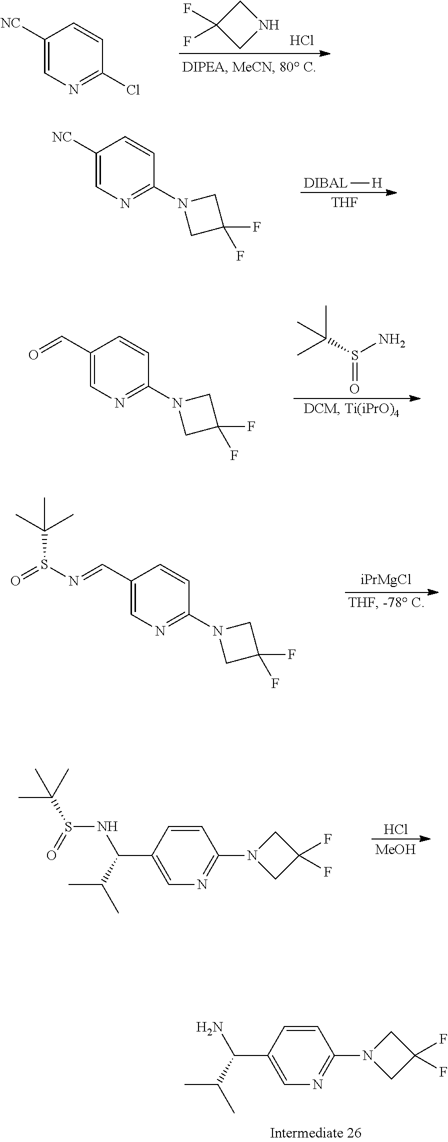

### Step 1: 6-(3,3-difluoroazetidin-1-yl)nicotinonitrile

A mixture of 6-chloronicotinonitrile (1.6 g, 11.55 mmol) and 3,3-difluoroazetidine hydrochloride (2.24 g, 17.32 mmol), DIPEA (6.1 ml, 34.64 mmol) in acetonitrile (100 mL) was stirred at 80° C. under a nitrogen atmosphere overnight. LCMS indicated the starting material was consumed completely and the desired product was formed. The reaction mixture was concentrated to give a residue, which was purified by column chromatography on silica gel (EtOAc in petroleum ether, from 20% to 30%) to yield 6-(3,3-difluoroazetidin-1-yl)nicotinonitrile (2.1 g, 93% yield) as a white solid. LC-MS: m/z 196 [M+H]⁺.

### Step 2: 6-(3,3-difluoroazetidin-1-yl)nicotinaldehyde

To a solution of 6-(3,3-difluoroazetidin-1-yl)nicotinonitrile (1.5 g, 7.68 mmol) in anhydrous THF (35 mL) was added DIBA1-H (13.36 ml, 1 M in toluene) dropwise at 0° C. After the addition was completed, the reaction mixture was stirred at this temperature for 1.5 hrs. LCMS showed the starting material was consumed completely and the desired product was formed. The reaction was quenched with 1 M H₂SO₄ solution (24 mL), neutralized with saturated aqueous NaHCO₃ solution (100 mL). The aqueous phase was extracted with DCM (100 ml×3). The combined organic layers were dried over Na₂SO₄, filtered, and concentrated. The residue was purified by column chromatography on silica gel (EtOAc in petroleum ether, from 10% to 25%) to give 6-(3,3-difluoroazetidin-1-yl)nicotinaldehyde (1 g, 66.67% yield) as a white solid. LC-MS m/z: 199 [M+H]⁺.

### Step 3: (R,E)-N-((6-(3,3-difluoroazetidin-1-yl)pyridin-3-yl)methylene)-2-methylpropane-2-sulfinamide

To a solution of 6-(3,3-difluoroazetidin-1-yl)nicotinaldehyde (1 g, 5.046 mmol) and (R)-2-methylpropane-2-sulfinamide (1.22 g, 10.09 mmol) in anhydrous DCM (30 mL) was added Ti(iPrO)₄ (2.88 g, 10.09 mmol) in one portion. The reaction mixture was stirred at room temperature overnight. LCMS showed the starting material was consumed completely and the desired product was formed. The reaction mixture was quenched by addition of water (20 mL) and a lot of white precipitate was appeared. After stirred for 30 min, the solid was filtered off and the filter cake was washed with DCM (30 ml×2). Then the organic phase of the filtrate was separated, dried over Na₂SO₄, filtered, and concentrated. The residue was purified by column chromatography on silica gel (EtOAc in petroleum ether, from 10% to 30%) to give (R,E)-N-((6-(3,3-difluoroazetidin-1-yl)pyridin-3-yl)methylene)-2-methylpropane-2-sulfinamide (1.4 g, 92% yield) as a white solid. LC-MS m/z: 302 [M+H]⁺.

### Step 4: (R)—N—((S)-1-(6-(3,3-difluoroazetidin-1-yl)pyridin-3-yl)-2-methylpropyl)-2-methylpropane-2-sulfinamide

To a stirred −78° C. solution of (R,E)-N-((6-(3,3-difluoroazetidin-1-yl)pyridin-3-yl)methylene)-2-methylpropane-2-sulfinamide (1.1 g, 3.65 mmol) in anhydrous THF (30 mL) was added iPrMgCl (7.3 ml, 14.6 mmol) dropwise over a period of 20 min. After the addition was completed, the reaction mixture was stirred at this temperature for 30 min LCMS showed the starting material was consumed completely and the desired product was formed. The reaction mixture was quenched with saturated aqueous NH₄Cl (10 mL), diluted with EtOAc (100 mL) and water (20 mL). The organic phase was washed with brine (30 ml×2), dried over Na₂SO₄, filtered, concentrated. The residue was purified by column chromatography on silica gel (DCM/EtOAc=1/3 to 1/1) to yield (R)—N—((S)-1-(6-(3,3-difluoroazetidin-1-yl)pyridin-3-yl)-2-methylpropyl)-2-methylpropane-2-sulfinamide (1 g, 87.26% yield) as a pale yellow solid. LC-MS m/z: 346 [M+H]⁺.

### Step 5: (S)-1-(6-(3,3-difluoroazetidin-1-yl)pyridin-3-yl)-2-methylpropan-1-amine

A solution of (R)—N—((S)-1-(6-(3,3-difluoroazetidin-1-yl)pyridin-3-yl)-2-methylpropyl)-2-methylpropane-2-sulfinamide (1 g, 2.89 mmol) in HCl/MeOH (30 ml, 4 M) was stirred at room temperature for 2 hrs. LCMS showed the starting material was consumed completely and the desired product was formed. The reaction solution was concentrated to give the residue, which was dissolved in DCM (30 mL). To the above solution was added saturated aqueous NaHCO₃ (0.5 mL), and solid NaHCO₃ (2 g), and the mixture was stirred at room temperature for 30 min. The mixture was filtered and the filtrate was dried over Na₂SO₄, and concentrated to give (S)-1-(6-(3,3-difluoroazetidin-1-yl)pyridin-3-yl)-2-methylpropan-1-amine (700 mg, 100% yield) as a pale yellow oil. LC-MS m/z: 242 [M+H]⁺.

The following intermediate was prepared according to procedure similar to that described for Intermediate 26 by using the morpholine as starting material.

| Intermediate | | |
|---|---|---|
| # | Structure | LC-MS |
| 27 | 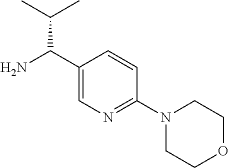 | 236 [M + H]⁺ |

### Intermediate 28

### (S)-3-(2-amino-3-methylbutyl)-1,2,4-oxadiazol-5(2H)-one, trifluoroacetate

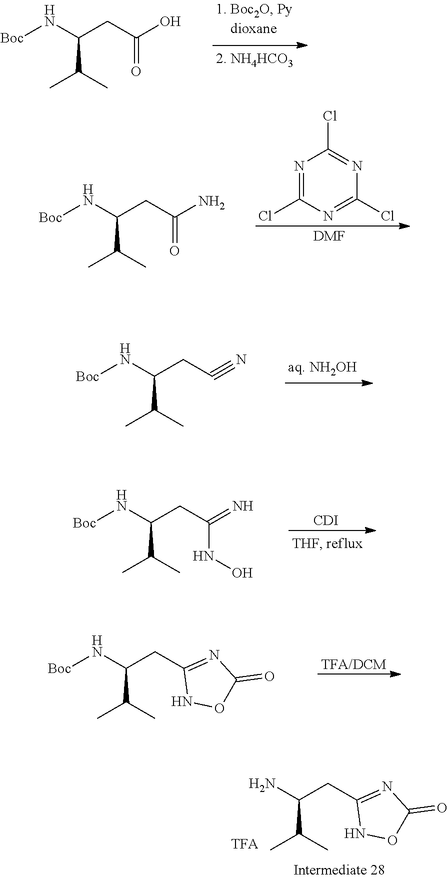

### Step 1: (S)-tert-butyl (1-amino-4-methyl-1-oxopentan-3-yl)carbamate

To a mixture of (S)-3-((tert-butoxycarbonyl)amino)-4-methylpentanoic acid (1.5 g, 6.49 mmol) in 1,4-dioxane (10 mL) was added di-tert-butyl dicarbonate (3.72 g, 17.06 mmol) followed by drop-wise addition of pyridine (0.32 mL, 4.02 mmol) at 0° C. The mixture was stirred at 0° C. for 10 min and ammonium bicarbonate (666.6 mg, 8.43 mmol) was added. The resulting mixture was stirred at room temperature overnight. The mixture was diluted with ethyl acetate and washed with water and brine, dried over Na₂SO₄ and concentrated to give (S)-tert-butyl (1-amino-4-methyl-1-oxopentan-3-yl)carbamate (1.4 g) as white solid. LC-MS: m/z=175 [M+H−56]⁺.

### Step 2: (S)-tert-butyl (1-cyano-3-methylbutan-2-yl)carbamate

To a solution of (S)-tert-butyl (1-amino-4-methyl-1-oxopentan-3-yl)carbamate (1.4 g, 6.08 mmol) in DMF (10 mL) was added 2,4,6-trichloro-1,3,5-triazine (1.68 g, 10.07 mmol) at 0° C. under a N₂ atmosphere. The resulting mixture was stirred at room temperature for 3 hrs. The mixture was diluted with ethyl acetate and washed with aqueous lithium chloride solution, dried over Na₂SO₄ and concentrated under reduced pressure to give a residue, which was purified by silica gel chromatography (petroleum ether:ethyl acetate=50:1 to 20:1) to give (S)-tert-butyl (1-cyano-3-methylbutan-2-yl)carbamate (1.12 g) as a white solid. LC-MS: m/z=157 [M+H−56]⁺. ¹H NMR (400 MHz, DMSO) δ 7.04 (m, 1H), 3.57-3.42 (m, 1H), 2.66 (m, 1H), 2.55-2.51 (m, 2H), 1.69 (m, 1H), 1.40 (s, 9H), 0.83 (m, 6H).

### Step 3: (S)-tert-butyl (1-(hydroxyamino)-1-imino-4-methylpentan-3-yl)carbamate

To a mixture of (S)-tert-butyl (1-cyano-3-methylbutan-2-yl)carbamate (1.12 g, 5.28 mmol) in ethanol (10 mL) was added aqueous hydroxylamine solution (1.56 mL, 26.38 mmol, 50% wt). The resulting mixture was stirred at 50° C. overnight. The mixture was then concentrated, diluted with ethyl acetate, and washed with water and brine. The organic layer was dried over Na₂SO₄ and concentrated to give a residue, which was purified by silica gel chromatography (dichloromethane:methanol=100:1 to 50:1) to give (S)-tert-butyl (1-(hydroxyamino)-1-imino-4-methylpentan-3-yl)carbamate (1.2 g) as a white solid. LC-MS: m/z=190 [M+H−56]⁺.

### Step 4: (S)-tert-butyl (3-methyl-1-(5-oxo-2,5-dihydro-1,2,4-oxadiazol-3-yl)butan-2-yl)carbamate

To a solution of (S)-tert-butyl (1-(hydroxyamino)-1-imino-4-methylpentan-3-yl)carbamate (1.2 g, 4.89 mmol) in THF (10 mL) was added triethylamine (0.34 mL, 2.45 mmol), and the mixture was heated to 70° C. A solution of N,N′-carbonyldiimidazole (1.20 g, 7.34 mmol) in THF (10 mL) was added to the mixture drop-wise, and the reaction was stirred at 70° C. overnight. The mixture was concentrated, diluted with ethyl acetate, and extracted with aqueous sodium hydroxide solution (10 mL, 1M). The layers were separated, and the aqueous layer was acidified with 2M hydrochloric acid. The acidified aqueous layer was extracted with dichloromethane twice, and the combined dichloromethane extracts were dried over Na₂SO₄, filtered and concentrated to give (S)-tert-butyl (3-methyl-1-(5-oxo-2,5-dihydro-1,2,4-oxadiazol-3-yl)butan-2-yl)carbamate (1.1 g) as a white solid. LC-MS: m/z=216 [M+H−56]⁺.

### Step 5: (S)-3-(2-amino-3-methylbutyl)-1,2,4-oxadiazol-5(2H)-one, trifluoroacetate

To a mixture of (S)-tert-butyl (3-methyl-1-(5-oxo-2,5-dihydro-1,2,4-oxadiazol-3-yl)butan-2-yl)carbamate (400 mg, 1.47 mmol) in dichloromethane (5 mL) was added trifluoroacetic acid (2.5 mL) drop-wise at 0° C. The reaction was stirred room temperature for 1 hr. The mixture was concentrated under reduced pressure to give (S)-3-(2-amino-3-methylbutyl)-1,2,4-oxadiazol-5(2H)-one trifluoroacetic acid salt (230 mg) as yellow syrup, which was directly used in the next reaction without purification. LC-MS: m/z=172 [M+H]⁺.

The following intermediates were prepared according to procedures similar to that described for Intermediate 28 by using the corresponding chiral acids.

| Intermediate | | |
|---|---|---|
| # | Structure | LC-MS |
| 29 | 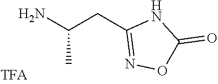 | 144 [M + H]⁺ |
| 30 | 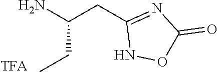 | 158 [M + H]⁺ |
| 31 | 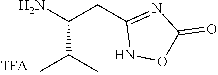 | 172 [M + H]⁺ |

### Intermediate 32

### (R)-3-methyl-1-(4H-1,2,4-triazol-3-yl)butan-2-amine

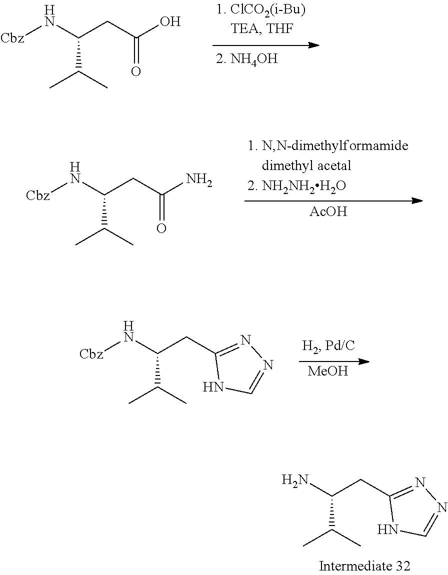

### Step 1: (R)-benzyl (1-amino-4-methyl-1-oxopentan-3-yl)carbamate

To a mixture of (R)-3-(benzyloxycarbonylamino)-4-methylpentanoic acid (5 g, 18.87 mmol) in anhydrous THF (70 mL) was added triethylamine (2.64 mL, 18.95 mmol) at 0° C., and the mixture was stirred 0° C. for 30 min. Ethyl chloroformate (1.81 mL, 18.95 mmol) was added to the mixture drop-wise, and the reaction was stirred for another 30 min Ammonium hydroxide (5.66 mL, 25% wt) was added, and the reaction was stirred at room temperature for 1 hr. The solvent was removed under vacuum, and the residue was combined with water and filtered. The filter cake was washed with water and dried under vacuum to give (R)-benzyl (1-amino-4-methyl-1-oxopentan-3-yl)carbamate (3.7 g) as a white solid. LC-MS (ESI) found: 265 [M+H]⁺.

### Step 2: (R)-benzyl (3-methyl-1-(4H-1,2,4-triazol-3-yl)butan-2-yl)carbamate

A solution of (R)-benzyl (1-amino-4-methyl-1-oxopentan-3-yl)carbamate (1.1 g, 4.17 mmol) in N,N-dimethylformamide dimethyl acetal (10 mL) was stirred at 100° C. for 0.5 hr. LCMS showed the starting material to be completely consumed. The mixture was concentrated, the residue was dissolved in acetic acid (10 mL), and hydrazine hydrate (0.5 mL, 8.00 mmol, 85% wt) was added to the mixture. The reaction was stirred at 90° C. for 1 hr and then poured into ice-water and filtered. The filter cake was washed with water and dried under vacuum to give (R)-benzyl (3-methyl-1-(4H-1,2,4-triazol-3-yl)butan-2-yl)carbamate (980 mg) as a white solid. LC-MS (ESI) found: 289 [M+H]⁺.

### Step 3: (R)-3-methyl-1-(4H-1,2,4-triazol-3-yl)butan-2-amine

A solution of (R)-benzyl (3-methyl-1-(4H-1,2,4-triazol-3-yl)butan-2-yl)carbamate (950 mg, 3.297 mmol) in methanol (20 mL) was degassed under a N₂ atmosphere three times, and Pd/C (130 mg, 10% wt) was added. The mixture was degassed again and stirred under a H₂ balloon at room temperature overnight. The mixture was filtered through Celite, and the filtrate was concentrated to give (R)-3-methyl-1-(4H-1,2,4-triazol-3-yl)butan-2-amine (600 mg) as a white solid. LC-MS (ESI) found: 155 [M+H]⁺.

### Intermediate 33

### (R)-5-(2-amino-3-methylbutyl)-1,3,4-oxadiazol-2-amine

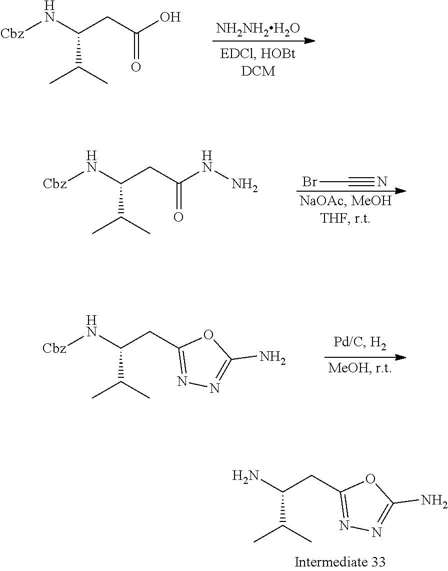

### Step 1: (R)-benzyl 1-hydrazinyl-4-methyl-1-oxopentan-3-ylcarbamate

To a solution of (R)-3-(benzyloxycarbonylamino)-4-methylpentanoic acid (1 g, 3.77 mmol) in dichloromethane (10 mL) was added 1-hydroxybenzotriazole (509 mg, 3.77 mmol), followed by the addition of 1-(3-dimethylaminopropyl)-3-ethylcarbodiimide, hydrochloride (795 mg, 4.14 mmol) in portions at 0° C. The mixture was stirred at 0° C. for 30 min and hydrazine hydrate (449 mg, 7.54 mmol, 85%) was added drop-wise. The reaction was stirred at room temperature for 16 hrs. The reaction mixture was then concentrated under vacuum, and the residue was combined with ice-water. The slurry was filtered, and the filter cake was washed with water and cold EtOH. The filter cake was then dried under vacuum to give (R)-benzyl 1-hydrazinyl-4-methyl-1-oxopentan-3-ylcarbamate (890 mg) as white solid. LC-MS (ESI) found: 280 [M+H]⁺.

### Step 2: (R)-benzyl 1-(5-amino-1,3,4-oxadiazol-2-yl)-3-methylbutan-2-ylcarbamate

To a solution of (R)-benzyl 1-hydrazinyl-4-methyl-1-oxopentan-3-ylcarbamate (950 mg, 3.40 mmol) in methanol (15 mL) and THF (5 mL) was added sodium acetate (837.46 mg, 10.21 mmol), followed by addition of cyanogen bromide (648.82 mg, 6.13 mmol) in portions at room temperature. The reaction was stirred at room temperature for 4 hrs and then concentrated under vacuum to give a residue. The residue was dissolved in ethyl acetate, washed with brine, dried over Na₂SO₄, filtered, and concentrated to give crude product. The crude product was purified by silica gel chromatography (petroleum ether:ethyl acetate=50:1 to 20:1) to give (R)-benzyl 1-(5-amino-1,3,4-oxadiazol-2-yl)-3-methylbutan-2-ylcarbamate (780 mg) as a white solid. LC-MS (ESI) found: 305 [M+H]. ¹H NMR (400 MHz, DMSO) δ 7.32 (m, 5H), 6.86 (s, 1H), 5.03-4.94 (m, 2H), 3.71-3.60 (m, 1H), 2.81 (m, 1H), 2.68 (m, 1H), 1.72 (m, 1H), 0.94-0.78 (m, 6H).

### Step 3: ((R)-5-(2-amino-3-methylbutyl)-1,3,4-oxadiazol-2-amine

To a solution of (R)-benzyl 1-(5-amino-1,3,4-oxadiazol-2-yl)-3-methylbutan-2-ylcarbamate (780 mg, 2.56 mmol) in methanol (20 mL) was added Pd/C (78 mg, 10%). The mixture was degassed with N₂ three times, degassed again and then stirred at room temperature under a H₂ atmosphere for 5 hrs. The mixture was filtered through Celite, and the filtrate was concentrated under reduced pressure to give (R)-5-(2-amino-3-methylbutyl)-1,3,4-oxadiazol-2-amine (350 mg) as a white solid. LC-MS (ESI) found: 171 [M+H]⁺.

The following intermediate was prepared according to a procedure similar to that described for Intermediate 33 by using methyl hydrazine.

| Intermediate | | |
|---|---|---|
| # | Structure | LC-MS |
| 34 | 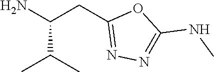 | 171 [M + H]⁺ |

### Intermediate 35

### (S)-5-(2-amino-3-methylbutyl)-N-methyl-1,3,4-thiadiazol-2-amine

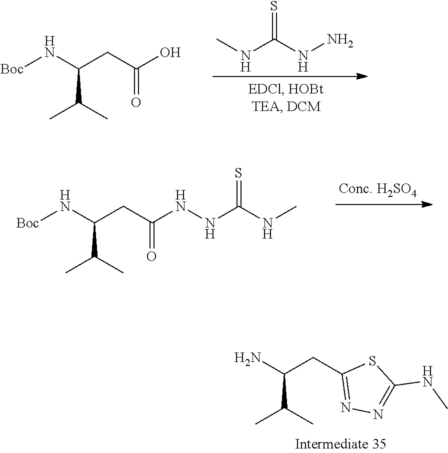

### Step 1: (S)-tert-butyl (4-methyl-1-(2-(methylcarbamothioyl)hydrazinyl)-1-oxopentan-3-yl)carbamate

To a mixture of (S)-3-(tert-butoxycarbonylamino)-4-methylpentanoic acid (540 mg, 2.04 mmol) and triethylamine (618.3 mg, 6.11 mmo) in dichloromethane (10 mL) was added HOBt (275.6 mg, 2.04 mmol), followed by addition of EDCI (391.1 mg, 2.04 mmol) in portions at 0° C. The mixture was stirred at 0° C. for 10 min, and N-methylhydrazinecarbothioamide (321.8 mg, 3.06 mmol) was added in one portion. The reaction was stirred at room temperature for 16 hrs. The mixture was diluted with dichloromethane and washed with saturated aqueous ammonium chloride solution, dried and concentrated to give crude product, which was purified by silica gel chromatography (petroleum ether:ethyl acetate=2:1) to give (S)-tert-butyl (4-methyl-1-(2-(methylcarbamothioyl)hydrazinyl)-1-oxopentan-3-yl)carbamate (250 mg) as a white solid. LC-MS (ESI) found: 263 [M+H−56]⁺.

### Step 2: (S)-5-(2-amino-3-methylbutyl)-N-methyl-1,3,4-thiadiazol-2-amine

(S)-tert-butyl (4-methyl-1-(2-(methylcarbamothioyl)hydrazinyl)-1-oxopentan-3-yl)carbamate (318 mg, 1 mmol) was added in portions to cooled concentrated sulfuric acid (5 mL) at 0° C. The reaction was stirred at room temperature for 16 hrs. The reaction mixture was poured into ice-water, and the mixture was made basic with concentrated sodium hydroxide solution. The mixture was extracted with chloroform/iso-propanol (3:1) three times. The combined organic layers were dried over anhydrous Na₂SO₄ and concentrated under reduced pressure to give (S)-5-(2-amino-3-methylbutyl)-N-methyl-1,3,4-thiadiazol-2-amine (140 mg) as a colorless oil. LC-MS (ESI) found: 201 [M+H]⁺.

The following intermediates were prepared according to procedures similar to that described for Intermediate 35 by using the corresponding chiral acids and hydrazinecarbothioamide.

| Intermediate | | |
|---|---|---|
| # | Structure | LC-MS |
| 36 | 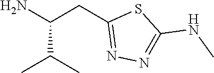 | 201 [M + H]⁺ |
| 37 | 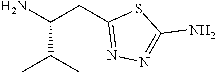 | 187 [M + H]⁺ |
| 38 | 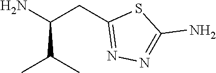 | 187 [M + H]⁺ |

### Intermediate 39

### (R)-2-methyl-2-(1-((1-methylpyrrolidin-2-yl)methyl)-1H-imidazol-4-yl)propan-1-amine

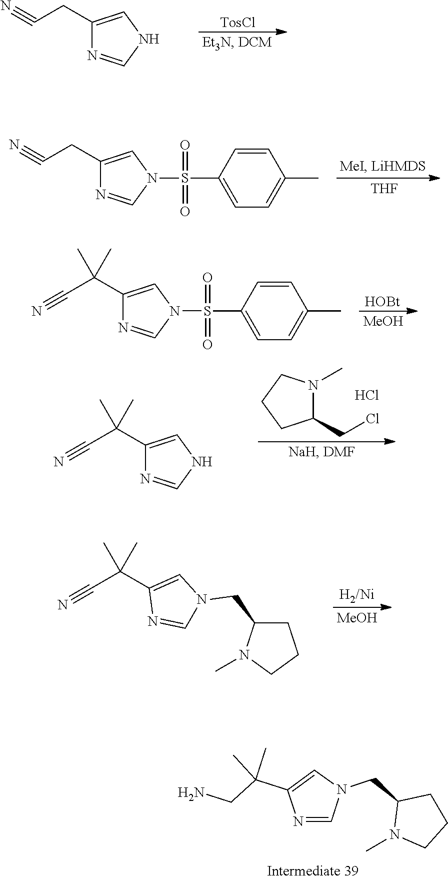

### Step 1: 2-(1-tosyl-1H-imidazol-4-yl)acetonitrile

To a solution of 2-(1H-imidazol-4-yl)acetonitrile (2 g, 18.69 mmol) in dichloromethane (25 mL) was added triethylamine (3.9 mL, 28.04 mmol), followed by drop-wise addition of a solution of 4-methylbenzene-1-sulfonyl chloride (4.6 g, 24.30 mmol) in dichloromethane (20 mL) at 0° C. The reaction was stirred at room temperature overnight. The mixture was washed with water and brine, dried over anhydrous Na₂SO₄ and concentrated to dryness. The residue was purified by silica gel chromatography (dichloromethane:methanol=100:1 to 20:1) to give 2-(1-tosyl-1H-imidazol-4-yl)acetonitrile (3.0 g) as a brown solid. LC-MS (ESI) found: 262 [M+H]⁺.

### Step 2: 2-methyl-2-(1-tosyl-1H-imidazol-4-yl)propanenitrile

To a solution of 2-(1-tosyl-1H-imidazol-4-yl)acetonitrile (2.5 g, 9.58 mmol) in THF (anhydrous, 60 mL) was added lithium bis(trimethylsilyl)amide (38.3 mL, 38.32 mmol, 1M in THF) drop-wise at −78° C. under a N₂ atmosphere. The mixture was stirred at −78° C. for 30 min, and iodomethane (3.0 mL, 47.90 mmol) was added drop-wise. The reaction was stirred at −78° C. for another 30 min. The reaction was quenched by drop-wise addition of water at −78° C. The mixture was diluted with ethyl acetate and washed with water and brine, dried over anhydrous Na₂SO₄ and concentrated under reduced pressure to give a residue, which was purified by silica gel chromatography (petroleum ether:ethyl acetate=1:1) to give 2-methyl-2-(1-tosyl-1H-imidazol-4-yl)propanenitrile (750 mg) as a white solid. LC-MS (ESI) found: 290 [M+H]±.

### Step 3: 2-(1H-imidazol-4-yl)-2-methylpropanenitrile

To a solution of 2-methyl-2-(1-tosyl-1H-imidazol-4-yl)propanenitrile (700 mg, 2.42 mmol) in methanol (15 mL) was added 1-hydroxybenzotriazole (654 mg, 4.844 mmol). The reaction mixture was stirred at room temperature overnight, and the solvent was removed under reduced pressure. The mixture was diluted with dilute hydrochloric acid and washed with ethyl acetate three times. The aqueous layer was basified by adding concentrated aqueous sodium bicarbonate solution. The basified aqueous layer was extracted with ethyl acetate three times. The combined ethyl acetate layers were dried over anhydrous Na₂SO₄ and concentrated under reduced pressure to give 2-(1H-imidazol-4-yl)-2-methylpropanenitrile (210 mg) as a yellow oil. LC-MS (ESI) found: 136 [M+H]⁺.

### Step 4: (R)-2-methyl-2-(1-((1-methylpyrrolidin-2-yl)methyl)-1H-imidazol-4-yl)propanenitrile

To a solution of 2-(1H-imidazol-4-yl)-2-methylpropanenitrile (210 mg, 1.55 mmol) in DMF (anhydrous, 20 mL) was added sodium hydride (187 mg, 4.67 mmol) in portions at 0° C. under a N₂ atmosphere. The mixture was stirred for 10 min and (R)-2-(chloromethyl)-1-methylpyrrolidine, hydrochloride (317 mg, 1.87 mmol) was added in portions at 0° C. under a N₂ atmosphere. The reaction was stirred at 30° C. for 2 hrs. The reaction was quenched with ice-water, and the mixture was extracted with ethyl acetate three times. The combined organic layers were washed with brine, dried over anhydrous Na₂SO₄ and concentrated under reduced pressure to give a residue, which was purified by silica gel chromatography (dichloromethane:methanol=100:1 to 20:1) to give (R)-2-methyl-2-(1-((1-methylpyrrolidin-2-yl)methyl)-1H-imidazol-4-yl)propanenitrile (210 mg) as yellow oil. LC-MS (ESI) found: 233 [M+H]⁺.

### Step 5: (R)-2-methyl-2-(1-((1-methylpyrrolidin-2-yl)methyl)-1H-imidazol-4-yl)propan-1-amine

A solution of (R)-2-methyl-2-(1-((1-methylpyrrolidin-2-yl)methyl)-1H-imidazol-4-yl)propanenitrile (200 mg, 0.86 mmol) in methanol (5 mL) was degassed three times under a N₂ atmosphere, and Raney-Ni (50 mg) was added. The reaction mixture was degassed again and stirred under a H₂ balloon at room temperature overnight. The mixture was filtered, and the filtrate was concentrated to dryness to give (R)-2-methyl-2-(1-((1-methylpyrrolidin-2-yl)methyl)-1H-imidazol-4-yl)propan-1-amine (200 mg, crude) as a colorless oil, which was directly used in the next reaction without purification. LC-MS (ESI) found: 237 [M+H]⁺.

### Intermediate 40

### 2-methyl-2-(4H-1,2,4-triazol-3-yl)propan-1-amine

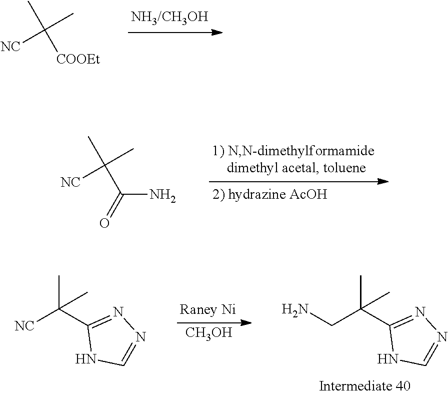

### Step 1: 2-cyano-2-methylpropanamide

A solution of ethyl 2-cyano-2-methylpropanoate (2.5 g, 17.7 mmol) in ammonia-methanol solution (30 mL, 1 M) was stirred at 30° C. overnight. The mixture was concentrated to dryness to give 2-cyano-2-methylpropanamide (1.90 g) as a white solid. The crude product was used in next step without further purification. ¹H NMR (400 MHz, CDCl₃) δ 6.28-6.20 (d, J=32 Hz, 2H), 1.54 (d, 6H).

### Step 2: 2-methyl-2-(4H-1,2,4-triazol-3-yl)propanenitrile

To a solution of 2-cyano-2-methylpropanamide (1.80 g, 16.1 mmol) in toluene (anhydrous, 10 mL) was added N,N-dimethylformamide dimethyl acetal (3.83 g, 32.1 mmol). The reaction was stirred at 100° C. for 2 hrs. The mixture was concentrated to dryness, and the residue was dissolved in acetic acid (10 mL), hydrazine hydrate (1.63 g, 32.1 mmol, 98% wt) was added to the mixture, and the reaction was stirred at 90° C. for 1 hr. Ice-water (100 mL) was added to the mixture, and the mixture was extracted with ethyl acetate three times. The combined organic layers were washed with brine, dried over Na₂SO₄, filtered and concentrated to give crude product, which was purified by silica gel chromatography (petroleum ether:ethyl acetate=3:1) to give 2-methyl-2-(4H-1,2,4-triazol-3-yl)propanenitrile (0.85 g) as a white solid. LC-MS (ESI) found: 137 [M+H]⁺. ¹H NMR (400 MHz, CDCl₃) δ 8.41 (d, 1H), 1.77 (d, 6H).

### Step 3: 2-methyl-2-(4H-1,2,4-triazol-3-yl)propan-1-amine

To a solution of 2-methyl-2-(4H-1,2,4-triazol-3-yl)propanenitrile (0.80 g, 5.88 mmol) in methanol (20 mL) was added Raney Ni (0.10 g). The mixture was degassed three times under a N₂ atmosphere and stirred under a H₂ balloon at room temperature overnight. The mixture was filtered, and the filtrate was concentrated to dryness to give 2-methyl-2-(4H-1,2,4-triazol-3-yl)propan-1-amine (780 mg) as a light blue solid. LC-MS (ESI) found: 141 [M+H]⁺.

### Intermediate 41

### (S)-1-(1-isobutyl-1H-imidazol-4-yl)-N,2-dimethylpropan-1-amine

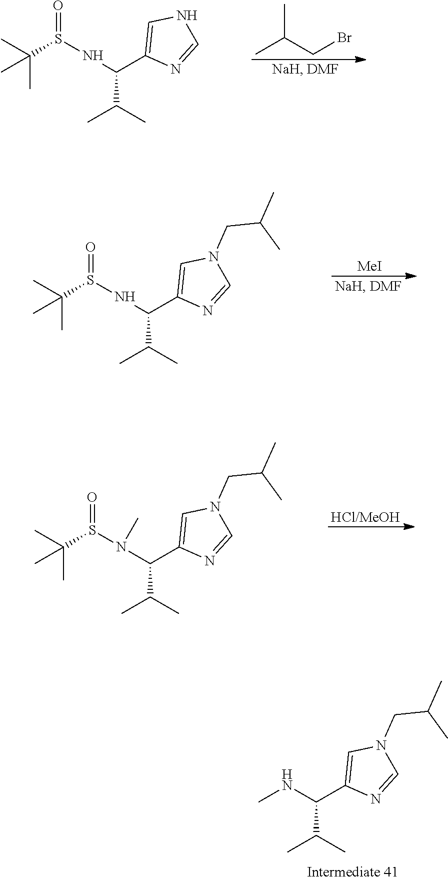

### Step 1: (S)—N—((S)-1-(1-isobutyl-1H-imidazol-4-yl)-2-methylpropyl)-2-methylpropane-2-sulfinamide

To a solution of (S)—N—((S)-1-(1H-imidazol-4-yl)-2-methylpropyl)-2-methylpropane-2-sulfinamide (2.15 g, 9.2 mmol) in N,N-dimethylformamide (8 mL) was added sodium hydride (662 mg, 27.6 mmol) at 0° C. The mixture was stirred at 0° C. for 30 min, followed by drop-wise addition of 1-bromo-2-methylpropane (1.63 g, 12.0 mmol) at 0° C. The reaction mixture was then stirred at room temperature overnight, quenched with NH₄Cl (sat.), and extracted with ethyl acetate twice. The combined organic layers were dried over anhydrous Na₂SO₄, concentrated under reduced pressure to give a residue. The residue was purified by silica gel chromatography (DCM:methanol=75:1 to 40:1) to give (S)—N—((S)-1-(1-isobutyl-1H-imidazol-4-yl)-2-methylpropyl)-2-methylpropane-2-sulfinamide (1.4 g, 53%) as a yellow oil. LC-MS (ESI) found: 300 [M+H]⁺.

### Step 2: (S)—N—((S)-1-(1-isobutyl-1H-imidazol-4-yl)-2-methylpropyl)-N,2-dimethylpropane-2-sulfinamide

To a solution of (S)—N—((S)-1-(1-isobutyl-1H-imidazol-4-yl)-2-methylpropyl)-2-methylpropane-2-sulfinamide (350 mg, 1.17 mmol) in N,N-dimethylformamide (8 mL) was added sodium hydride (56 mg, 2.34 mmol) at 0° C., and the mixture was stirred at 0° C. for 30 min Iodomethane (216 mg, 1.52 mmol) was then added at 0° C., and the reaction mixture was stirred at room temperature overnight. After quenching the reaction with NH₄Cl (sat.), the reaction mixture was extracted with ethyl acetate twice. The combined organic layers were dried over anhydrous Na₂SO₄ and concentrated under reduced pressure to give a residue. The residue was purified by silica gel chromatography (DCM:methanol=30:1) to give (S)—N—((S)-1-(1-isobutyl-1H-imidazol-4-yl)-2-methylpropyl)-N,2-dimethylpropane-2-sulfinamide (180 mg, 49%) as a yellow oil. LC-MS (ESI) found: 314 [M+1]⁺.

### Step 3: (S)-1-(1-isobutyl-1H-imidazol-4-yl)-N,2-dimethylpropan-1-amine

A solution of (S)—N—((S)-1-(1-isobutyl-1H-imidazol-4-yl)-2-methylpropyl)-N,2-dimethylpropane-2-sulfinamide (180 mg, 0.57 mmol) in HCl-methanol solution (10 mL, 1M) was stirred at room temperature overnight. The reaction solution was then adjusted to pH=8 with NaHCO₃ (sat.) and extracted twice with DCM. The combined organic layers were washed with water and brine (sat.), dried over Na₂SO₄, filtered and concentrated to dryness to give (S)-1-(4-(benzyloxy)pyridine-2-yl)-2-methylpropan-1-amine (160 mg, crude) as colorless oil, which was used directly in the next step without purification. LC-MS (ESI) found: 210 [M+H]⁺.

### EXAMPLES

### General Procedure for Coupling of Intermediates

A general procedure for preparation of compounds of the present disclosure is provided below using Example 1 as an illustration.

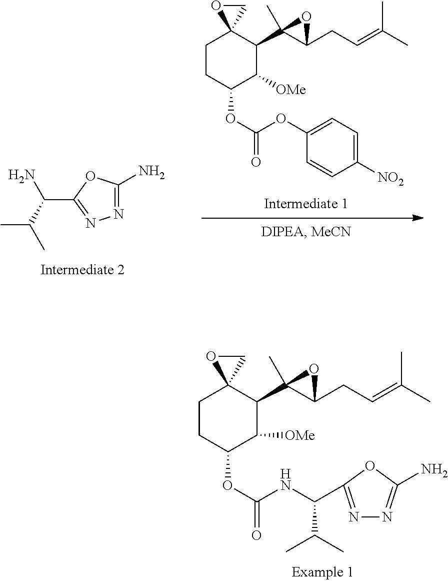

To a mixture of an appropriate intermediate compound in CH₃CN was added DIPEA drop-wise at 0-5° C. The mixture was then stirred at 0-5° C. for 10 min, and carbonate Intermediate 1 was added to the mixture in portions at 0° C. under a N₂ atmosphere. The reaction mixture was stirred at 25° C. until the reaction was complete as judged by TLC. The solvent was removed under vacuum below 40° C. The residue was diluted with DCM, and the DCM diluent was washed with ammonium acetate buffer (pH-4). The combined aqueous layers were back-extracted with DCM. The combined organic layers were washed with aq. NaHCO₃ solution, dried over Na₂SO₄ and concentrated. Purification by silica gel column chromatography (DCM:MeOH=100:0 to 60:1), followed by preparative HPLC (Method B, H₂O (0.1% FA)/CH₃CN) gave the title compound. Characterization data are provided below.

The following compounds were also prepared according to procedures similar to that described for Example 1 by using the corresponding intermediates.

| Example | Int. | Structure | LC-MS | H-NMR (400 MHz, CDCl₃) |
|---|---|---|---|---|
| 1 | 2 | 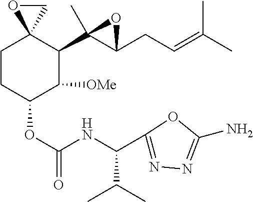 [SMILES: CO[C@@H]1[C@H](OC(=O)N[C@H](c2nnc(N)o2)C(C)C)CC[C@]2(CO2)[C@H]1[C@@]1(C)O[C@@H]1CC=C(C)C] | 465 [M + H]⁺ | 5.55-5.75 (d, J = 9.6 Hz, 1H), 5.49 (s, 1H), 5.33 (s, 2H), 5.17-5.20 (t, 1H), 4.71 (t, 1H), 3.61-3.64 (dd, J = 2.4 Hz, J = 11.2 Hz, 1H), 3.39 (s, 3H), 2.97-2.98 (d, J = 4 Hz, 1H), 2.54- 2.61 (m, 2H), 2.35-2.39 (m, 1H), 1.96-2.22 (m, 5H), 1.78-1.94 (m, 3H), 1.74 (s, 3H), 1.65 (s, 3H), 1.19 (s, 3H), 0.98-1.09 (m, 7H) |
| 2 | 3 | 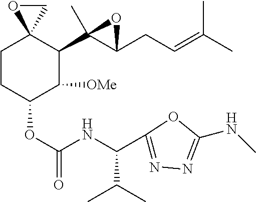 [SMILES: CNc1nnc([C@@H](NC(=O)O[C@@H]2CC[C@]3(CO3)[C@@H]([C@@]3(C)O[C@@H]3CC=C(C)C)[C@@H]2OC)C(C)C)o1] | 479 [M + H]⁺ | 5.40 (s, 1H), 4.98-5.14 (m, 3H), 4.63- 4.66 (t, 3H), 3.53-3.56 (dd, J = 7.2 Hz, 2.4 Hz, 1H), 3.31 (s, 3H), 2.93- 2.94 (d, J = 4.8 Hz, 3H), 2.89-2.90 (d, J = 4.4 Hz, 1H), 2.50-2.54 (t, 3H), 2.48-2.49 (d, J = 4.0 Hz, 1H), 2.26- 2.31 (m, 1H), 2.05-2.16 (m, 2H), 1.85-2.00 (m, 3H), 1.75-1.79 (m, 2H), 1.67 (s, 3H), 1.58 (s, 3H), 1.12 (s, 3H), 1.00-1.03 (d, J = 12.0 Hz, 1H), 0.90-0.94 (t, 6H) |
| 3 | 4 | 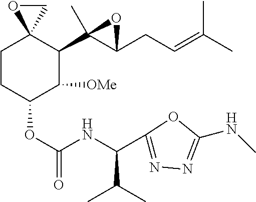 [SMILES: CNc1nnc([C@H](NC(=O)O[C@@H]2CC[C@]3(CO3)[C@@H]([C@@]3(C)O[C@@H]3CC=C(C)C)[C@@H]2OC)C(C)C)o1] | 479 [M + H]⁺ | 7.49 (m, 1H), 7.14 (s, 1H), 5.27-5.28 (s, 1H), 5.19-5.22 (t, 1H), 4.43 (m, 1H), 3.54-3.56 (d, J = 8.0 Hz, 1H), 3.29 (s, 3H), 2.82-2.83 (d, J = 4.8 Hz, 1H), 2.78-2.79 (d, J = 4.8 Hz, 3H), 2.55-2.56 (m, 2H), 2.17-2.21 (t, 2H), 2.09-2.12 (m, 1H), 1.78-1.93 (m, 3H), 1.72 (s, 3H), 1.70-1.72 (m, 1H), 1.63 (s, 3H), 1.10-1.13 (m, 1H), 1.12 (s, 3H), 0.95-0.97 (d, J = 6.8 Hz, 3H), 0.86-0.88 (d, J = 6.8 Hz, 3H) |
| 4 | 5 | 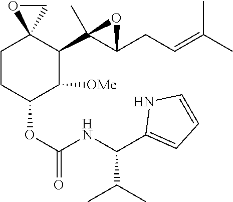 [SMILES: CO[C@@H]1[C@H](OC(=O)N[C@H](c2ccc[nH]2)C(C)C)CC[C@]2(CO2)[C@H]1[C@@]1(C)O[C@@H]1CC=C(C)C] | 448 [M + H]⁺ | 6.92 (s, 2H), 5.45 (s, 1H), 2.26 (d, J = 8.8 Hz, 1H), 5.12 (t, J = 6.8 Hz, 1H), 4.62 (br, 1H), 3.60 (d, J = 5.6 Hz, 1H), 3.34 (s, 3H), 2.90 (d, J = 4.4 Hz, 1H), 2.50-2.56 (m, 3H), 2.28-2.35 (m, 1H), 1.77-2.12 (m, 6H), 1.68 (s, 3H), 1.59 (s, 3H), 1.18 (s, 3H), 1.01 (d, J = 13.6 Hz, 1H), 0.80-0.92 (m, 6H) |
| 5 | 6 | 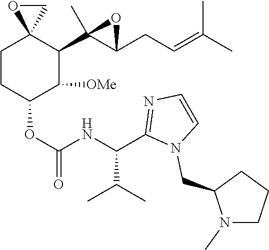 [SMILES: CO[C@@H]1[C@H](OC(=O)N[C@H](c2nccn2C[C@H]2CCCN2C)C(C)C)CC[C@]2(CO2)[C@H]1[C@@]1(C)O[C@@H]1CC=C(C)C] | 545 [M + H]⁺ | 6.97 (s, 2H), 5.76 (d, J = 8.8 Hz, 1H), 5.47 (s, 1H), 5.18 (t, J = 7.2 Hz, 1H), 4.62 (d, J = 9.2 Hz, 1H), 4.22 (m, 1H), 3.80-3.85 (m, 1H), 3.59 (dd, J = 2.4 Hz, J = 16 Hz, 4H), 3.37 (s, 3H), 3.17 (m, 2H), 2.96 (d, J = 4.0 Hz, 1H), 2.66 (s, 1H), 2.50-2.54 (m, 2H), 2.18-2.28 (m, 6H), 1.92-2.10 (m, 4H), 1.95-1.98 (m, 1H), 1.75-1.83 (m, 3H), 1.66 (s, 3H), 1.57 (m, 3H), 1.10 (s, 3H), 0.97 (d, J = 6.8 Hz, 3H), 1.00 (d, J = 6.8 Hz, 1H) |
| 6 | 7 | 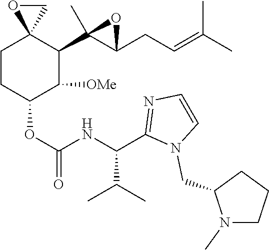 [SMILES: CO[C@@H]1[C@H](OC(=O)N[C@H](c2nccn2C[C@@H]2CCCN2C)C(C)C)CC[C@]2(CO2)[C@H]1[C@@]1(C)O[C@@H]1CC=C(C)C] | 545 [M + H]⁺ | 7.17 (d, J = 9.7 Hz, 1H), 7.02 (d, J = 1.1 Hz, 1H), 6.95 (d, J = 1.2 Hz, 1H), 5.40 (s, 1H), 5.11 (m, 1H), 4.47 (m, 1H), 4.40-4.29 (m, 2H), 3.51 (m, 2H), 3.29 (s, 3H), 3.23-3.12 (m, 1H), 2.89 (d, J = 4.2 Hz, 1H), 2.67 (s, 3H), 2.59 (m, 1H), 2.49 (m, 2H), 2.31- 2.19 (m, 2H), 2.17-2.02 (m, 2H), 1.95-1.70 (m, 7H), 1.66 (s, 3H), 1.57 (s, 3H), 1.10 (s, 3H), 1.00 (d, J = 6.6 Hz, 4H), 0.70 (d, J = 6.6 Hz, 3H) |
| 7 | 8 | 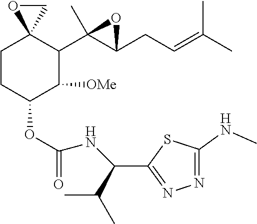 [SMILES: CNc1nnc([C@H](NC(=O)O[C@@H]2CC[C@]3(CO3)C([C@@]3(C)O[C@@H]3CC=C(C)C)[C@@H]2OC)C(C)C)s1] | 495 [M + H]⁺ | 5.39-5.45 (m, 2H), 5.12-5.16 (m, 1H), 5.02 (s, 1H), 4.76-4.80 (m, 1H), 3.56-3.59 (m, 1H), 3.37 (s, 3H), 2.97 (s, 3H), 2.90 (d, J = 4.4 Hz, 1H), 2.49-2.52 (m, 1H), 2.26 (d, J = 4.4 Hz, 1H), 2.27-2.33 (m, 1H), 1.87- 2.17 (m, 5H), 1.72-1.76 (m, 1H), 1.67 (s, 3H), 1.59 (s, 3H), 1.14 (s, 3H), 0.90-0.98 (m, 7H) |
| 8 | 9 | 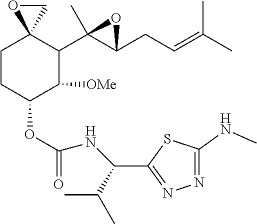 [SMILES: CNc1nnc([C@@H](NC(=O)O[C@@H]2CC[C@]3(CO3)C([C@@]3(C)O[C@@H]3CC=C(C)C)[C@@H]2OC)C(C)C)s1] | 495 [M + H]⁺ | 5.39-5.48 (m, 2H), 5.21 (s, 1H), 5.11- 5.14 (m, 1H), 4.68-4.70 (m, 1H), 3.57 (d, J = 10.8 Hz, 1H), 3.35 (s, 3H), 2.96 (s, 3H), 2.90 (d, J = 4.4 Hz, 1H), 2.47-2.51 (m, 2H), 2.26-2.32 (m, 2H), 1.94-2.12 (m, 3H), 1.88 (d, J = 10.8 Hz, 1H), 1.71-1.82 (m, 1H), 1.67 (s, 3H), 1.58 (s, 3H), 1.13 (s, 3H), 0.97-1.03 (m, 1H), 0.90-0.93 (m, 6H) |
| 9 | 10 | 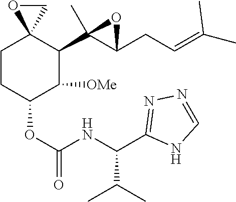 [SMILES: CO[C@@H]1[C@H](OC(=O)N[C@H](c2nnc[nH]2)C(C)C)CC[C@]2(CO2)[C@H]1[C@@]1(C)O[C@@H]1CC=C(C)C] | 449 [M + H]⁺ | 8.03 (s, 1H), 5.62 (d, J = 56.4 Hz, 2H), 5.19 (s, 1H), 4.97-4.77 (m, 1H), 3.69 (d, J = 9.6 Hz, 1H), 3.41 (s, 3H), 2.98 (d, J = 4.2 Hz, 1H), 2.63- 2.54 (m, 2H), 2.41 (dd, J = 14.3, 6.8 Hz, 2H), 2.22-1.80 (m, 5H), 1.75 (s, 3H), 1.66 (s, 3H), 1.21 (s, 3H), 1.09 (d, J = 11.9 Hz, 1H), 0.98 (d, J = 6.7 Hz, 3H), 0.89 (d, J = 6.8 Hz, 3H) |
| 10 | 11 | 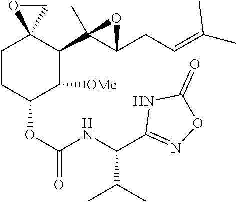 [SMILES: CO[C@@H]1[C@H](OC(=O)N[C@H](c2noc(=O)[nH]2)C(C)C)CC[C@]2(CO2)[C@H]1[C@@]1(C)O[C@@H]1CC=C(C)C] | 466 [M + H]⁺ | 5.66-5.42 (m, 2H), 5.10 (s, 1H), 4.51-4.37 (m, 1H), 3.62 (d, J = 11.4 Hz, 1H), 3.38 (s, 3H), 2.88 (d, J = 3.9 Hz, 1H), 2.64 (s, 1H), 2.49 (dd, J = 14.3, 5.2 Hz, 2H), 2.35-2.16 (m, 2H), 1.97 (ddt, J = 45.6, 25.3, 9.9 Hz, 5H), 1.68 (s, 3H), 1.58 (s, 3H), 1.13 (d, J = 15.0 Hz, 3H), 0.98 (dd, J = 22.0, 10.1 Hz, 7H) |
| 11 | 12 | 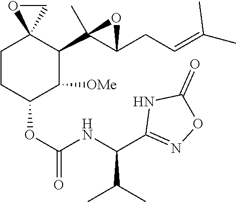 [SMILES: CO[C@@H]1[C@H](OC(=O)N[C@@H](c2noc(=O)[nH]2)C(C)C)CC[C@]2(CO2)[C@H]1[C@@]1(C)O[C@@H]1CC=C(C)C] | 466 [M + H]⁺ | 5.42 (d, J = 29.9 Hz, 1H), 5.11 (t, J = 7.1 Hz, 1H), 4.33 (s, 1H), 3.60 (d, J = 10.1 Hz, 1H), 3.51-3.27 (m, 3H), 2.90 (s, 1H), 2.53 (dd, J = 13.7, 7.8 Hz, 2H), 2.32 (s, 1H), 2.25-1.73 (m, 6H), 1.68 (s, 3H), 1.59 (s, 3H), 1.15 (s, 3H), 1.10-0.87 (m, 7H) |
| 12 | 13 | 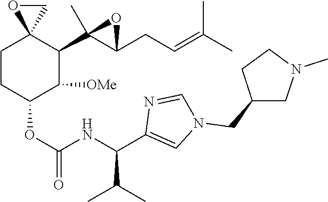 [SMILES: CO[C@@H]1[C@H](OC(=O)N[C@@H](c2cn(C[C@H]3CCN(C)C3)cn2)C(C)C)CC[C@]2(CO2)[C@H]1[C@@]1(C)O[C@@H]1CC=C(C)C] | 545 [M + H]⁺ | 7.70 (s, 1H), 6.90 (d, J = 35.3 Hz, 1H), 5.89 (d, J = 9.3 Hz, 0.6H), 5.64- 5.59 (m, 0.4H), 5.46 (s, 1H), 5.24- 5.21 (m, 1H), 4.51-4.32 (m, 1H), 4.13-4.03 (m, 2H), 3.65-3.62 (m, 1H), 3.54-3.38 (m, 3H), 3.34-3.17 (m, 2H), 3.05-2.89 (m, 3H), 2.77 (d, J = 10.8 Hz, 3H), 2.68-2.49 (m, 2H), 2.40-2.35 (m, 1H), 2.29-1.82 (m, 7H), 1.77 (d, J = 5.8 Hz, 3H), 1.67 (s, 3H), 1.19 (d, J = 27.7 Hz, 3H), 1.12-0.96 (m, 4H), 0.88-0.80 (m, 3H) |
| 13 | 14 | 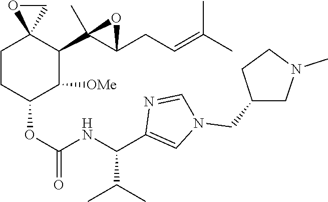 [SMILES: CO[C@@H]1[C@H](OC(=O)N[C@H](c2cn(C[C@@H]3CCN(C)C3)cn2)C(C)C)CC[C@]2(CO2)[C@H]1[C@@]1(C)O[C@@H]1CC=C(C)C] | 545 [M + H]⁺ | 7.33 (t, 1H), 6.69 (d, J = 4.4 Hz, 1H), 5.56-5.29 (m, 2H), 5.13 (t, 1H), 4.38 (dd, J = 8.5, 6.8 Hz, 1H), 3.79 (d, J = 8.0 Hz, 2H), 3.61-3.46 (m, 1H), 3.34 (s, 2H), 2.90 (d, J = 4.3 Hz, 1H), 2.60-2.34 (m, 6H), 2.31-2.21 (m, 5H), 2.14-1.88 (m, 6H), 1.85 (d, J = 11.2 Hz, 1H), 1.74 (t, 1H), 1.66 (d, J = 7.6 Hz, 3H), 1.58 (s, 3H), 1.43- 1.39 (m, 1H), 1.12 (s, 2H), 0.99 (d, J = 13.2 Hz, 1H), 0.86-0.72 (m, 6H) |
| 14 | 15 | 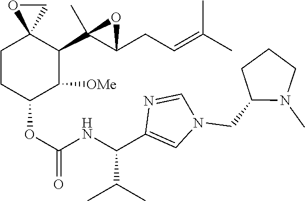 [SMILES: CO[C@@H]1[C@H](OC(=O)N[C@H](c2cn(C[C@@H]3CCCN3C)cn2)C(C)C)CC[C@]2(CO2)[C@H]1[C@@]1(C)O[C@@H]1CC=C(C)C] | 545 [M + H]⁺ | 7.62 (s, 1H), 6.90 (s, 1H), 5.75 (d, J = 9.1 Hz, 1H), 5.52 (s, 1H), 5.21 (m, 1H), 4.54-4.44 (m, 1H), 4.21 (m, 1H), 4.01 (m, 1H), 3.63 (m, 1H), 3.42 (s, 3H), 3.35 (m, 1H), 2.99 (d, J = 4.3 Hz, 1H), 2.93-2.82 (m, 1H), 2.58 (m, 2H), 2.50-2.32 (m, 5H), 2.27- 2.10 (m, 3H), 1.99 (m, 3H), 1.83 (m, 3H), 1.76 (s, 3H), 1.67 (m, 4H), 1.21 (s, 3H), 1.08 (d, J = 12.5 Hz, 1H), 0.92 (d, J = 6.7 Hz, 3H), 0.85 (d, J = 6.7 Hz, 3H) |
| 15 | 16 | 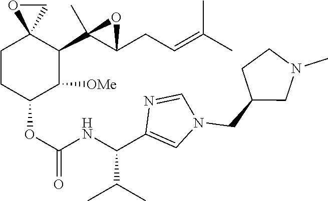 [SMILES: CO[C@@H]1[C@H](OC(=O)N[C@H](c2cn(C[C@H]3CCN(C)C3)cn2)C(C)C)CC[C@]2(CO2)[C@H]1[C@@]1(C)O[C@@H]1CC=C(C)C] | 545 [M + H]⁺ | 7.54 (d, J = 23.9 Hz, 1H), 6.78 (d, J = 14.7 Hz, 1H), 5.74 (d, J = 9.2 Hz, 1H), 5.45 (s, 1H), 5.12 (t, J = 7.1 Hz, 1H), 4.44-4.33 (m, 1H), 4.11-3.99 (m, 1H), 3.91 (dd, J = 13.8, 6.0 Hz, 1H), 3.54 (dd, J = 11.3, 2.4 Hz, 1H), 3.36 (d, J = 21.0 Hz, 4H), 3.11 (s, 1H), 2.90 (td, J = 20.1, 9.6 Hz, 4H), 2.65 (s, 3H), 2.54-2.45 (m, 2H), 2.28 (dd, J = 14.2, 6.5 Hz, 1H), 2.19- 2.03 (m, 4H), 1.96-1.83 (m, 2H), 1.78 (s, 1H), 1.67 (s, 3H), 1.58 (s, 3H), 1.18 (s, 1H), 1.11 (s, 3H), 1.00 |
| | | | | (d, J = 7.6 Hz, 1H), 0.84 (d, J = 6.7 |
| | | | | Hz, 3H), 0.76 (d, J = 6.6 Hz, 3H) |
| 16 | 17 | 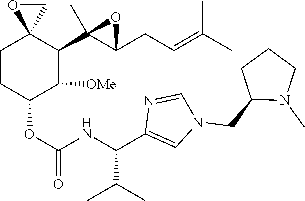 [SMILES: CO[C@@H]1[C@H](OC(=O)N[C@H](c2cn(C[C@H]3CCCN3C)cn2)C(C)C)CC[C@]2(CO2)[C@H]1[C@@]1(C)O[C@@H]1CC=C(C)C] | 545 [M + H]⁺ | 7.70 (s, 1H), 6.90 (s, 1H), 5.80-5.78 (d, J = 8 Hz, 1H), 5.43 (s, 1H), 5.13- 5.10 (t, 1H), 4.47-4.35 (m, 2H), 4.08-4.03 (m, 1H), 3.56-3.49 (m, 2H), 3.31 (s, 3H), 3.07-3.06 (t, 1H), 2.91-2.90 (d, J = 4 Hz, 1H), 2.54- 2.48 (m, 3H), 2.41 (s, 3H), 2.34- 2.27 (m, 1H), 2.23-1.98 (m, 4H), 1.90-1.74 (m, 6H), 1.67 (s, 3H), 1.58 (s, 3H), 1.11 (s, 3H), 1.01-0.98 (d, J = 12 Hz, 1H), 0.83-0.78 (m, 6H) |
| 17 | 18 | 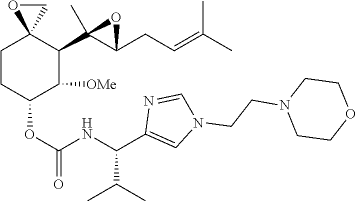 [SMILES: CO[C@@H]1[C@H](OC(=O)N[C@H](c2cn(CCN3CCOCC3)cn2)C(C)C)CC[C@]2(CO2)[C@H]1[C@@]1(C)O[C@@H]1CC=C(C)C] | 561 [M + H]⁺ | 7.40 (s, 1H), 6.74 (s, 1H), 5.39 (m, 2H), 5.13 (m, 1H), 4.39 (m, 1H), 3.92 (m, 2H), 3.67-3.60 (m, 4H), 3.54 (m, 1H), 3.33 (s, 3H), 2.90 (d, J = 4.3 Hz, 1H), 2.62 (m, 2H), 2.52-2.37 (m, 5H), 2.29 (m, 1H), 2.10 (m, 4H), 1.86 (d, J = 11.2 Hz, 1H), 1.71 (s, 4H), 1.67 (s, 3H), 1.12 (s, 3H), 0.97 (m, 1H), 0.84 (d, J = 6.7 Hz, 3H), 0.77 (d, J = 6.7 Hz, 3H) |
| 18 | 19 | 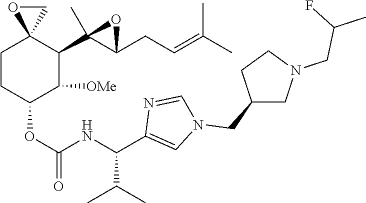 | 595 [M + H]⁺ | 7.43 (d, J = 20.4 Hz, 1H), 6.72 (s, 1H), 5.80 (t, J = 56.0 Hz, 1H), 5.43 (s, 1H), 5.13 (s, 1H), 4.44-4.33 (m, 1H), 3.83 (dd, J = 19.1, 10.5 Hz, 2H), 3.54 (dd, J = 11.2, 2.6 Hz, 1H), 3.31 (d, J = 14.6 Hz, 3H), 2.90 (d, J = 4.3 Hz, 1H), 2.79 (td, J = 14.9, 4.1 Hz, 3H), 2.53 (ddd, J = 18.5, 16.2, 6.5 Hz, 5H), 2.33-2.21 (m, 1H), 2.08 (ddd, J = 27.4, 16.9, 9.6 Hz, 3H), 1.98-1.83 (m, 3H), 1.76 (d, J = 14.3 Hz, 1H), 1.67 (s, 3H), 1.58-1.53 (m, 3H), 1.44 (d, J = 7.2 Hz, 2H), 1.12 (s, |
| | | | | 3H), 0.99 (d, J = 13.9 Hz, 1H), 0.85 |
| | | | | (d, J = 6.7 Hz, 3H), 0.77 (d, J = 6.6 |
| | | | | Hz, 3H) |
| 19 | 20 | 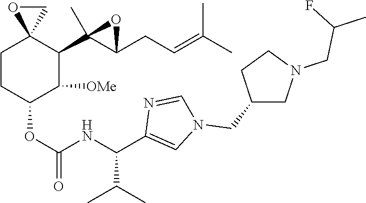 | 595 [M + H]⁺ | 7.39 (s, 1H), 6.76 (s, 1H), 5.84 (m, 1H), 5.53 (d, J = 9.0 Hz, 1H), 5.47 (s, 1H), 5.19 (t, J = 7.4 Hz, 1H), 4.44 (dd, J = 8.8, 6.9 Hz, 1H), 3.85 (dd, J = 10.8, 4.6 Hz, 2H), 3.60 (dd, J = 11.2, 2.7 Hz, 1H), 3.37 (s, 3H), 2.96 (d, J = 4.3 Hz, 1H), 2.88-2.76 (m, 3H), 2.59 (m, 5H), 2.50-2.43 (m, 1H), 2.39-2.29 (m, 1H), 2.23-2.08 (m, 3H), 2.07-1.96 (m, 2H), 1.73 (s, 3H), 1.64 (s, 3H), 1.55-1.41 (m, 1H), 1.18 (s, 3H), 1.05 (d, J = 12.4 Hz, 1H), 0.90 (d, J = 6.8 Hz, 3H), |
| | | | | 0.83 (d, J = 6.7 Hz, 3H) |
| 20 | 21 | 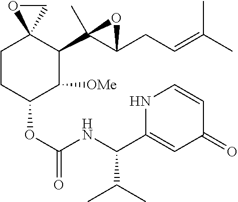 [SMILES: CO[C@@H]1[C@H](OC(=O)N[C@H](c2cc(=O)cc[nH]2)C(C)C)CC[C@]2(CO2)[C@H]1[C@@]1(C)O[C@@H]1CC=C(C)C] | 475 [M + H]⁺ | 7.73 (s, 1H), 6.47 (s, 2H), 5.53-5.61 (m, 2H), 5.19-5.22 (m, 1H), 4.46 (s, 1H), 3.65-3.68 (m, 1H), 3.33 (s, 3H), 2.99 (d, J = 4.4 Hz, 1H), 2.63-2.66 (m, 1H), 2.59 (d, J = 3.6 Hz, 1H), 2.30-2.44 (m, 3H), 2.10-2.21 (m, 2H), 1.98-2.05 (m, 2H), 1.85-1.93 (m, 1H), 1.77 (s, 3H), 1.68 (s, 3H), 1.21 (s, 3H), 1.11 (d, J = 12.8 Hz, 1H), 0.99 (d, J = 6.0 Hz, 3H), 0.90- 0.92 (m, 3H) |
| 21 | 22 | 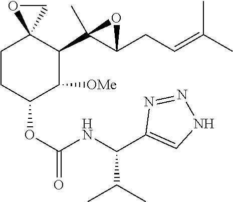 [SMILES: CO[C@@H]1[C@H](OC(=O)N[C@H](c2c[nH]nn2)C(C)C)CC[C@]2(CO2)[C@H]1[C@@]1(C)O[C@@H]1CC=C(C)C] | 449 [M + H]⁺ | 7.52 (s, 1H), 5.70 (d, J = 8.8 Hz, 1H), 5.48 (s, 1H), 5.18 (s, 1H), 4.85-4.62 (m, 1H), 3.68 (d, J = 11.8 Hz, 1H), 3.40 (s, 3H), 2.98 (d, J = 4.2 Hz, 1H), 2.56-2.62 (m, 2H), 2.35-2.40 (m, 1H), 2.09-2.20 (m, 4H), 2.10 (d, J = 11.2 Hz, 1H), 1.81-1.88 (m, 2H), 1.74 (s, 3H), 1.22 (s, 3H), 1.10 (d, J = 12 Hz, 1H), 0.91 (dd, J = 6.8 Hz, 13.6 Hz, 3H) |
| 22 | 23 | 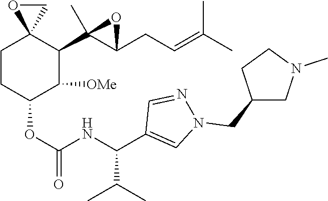 [SMILES: CO[C@@H]1[C@H](OC(=O)N[C@H](c2cnn(C[C@H]3CCN(C)C3)c2)C(C)C)CC[C@]2(CO2)[C@H]1[C@@]1(C)O[C@@H]1CC=C(C)C] | 545 [M + H]⁺ | 7.36 (s, 2H), 5.43 (s, 1H), 5.13 (t, J = 7.2 Hz, 1H), 4.48 (t, J = 6.4 Hz, 1H), 4.11 (m, 1H), 3.57 (d, J = 6.4 Hz, 1H), 3.37 (s, 3H), 3.63 (m, 1H), 3.20 (s, 1H), 3.00-3.08 (m, 3H), 2.89 (d, J = 4.4 Hz, 1H), 2.72-2.82 (m, 3H), 2.64 (s, 3H), 2.47-2.53 (m, 3H), 2.28-2.32 (m, 1H), 2.06-2.17 (m, 2H), 1.88-2.01 (m, 3H), 1.74-1.81 (m, 2H), 1.67 (s, 3H), 1.59 (s, 3H), 1.14 (s, 2H), 0.97 (d, J = 2.8 Hz, 1H), 0.83 (dd, J = 7.2 Hz, 6H) |
| 23 | 24 | 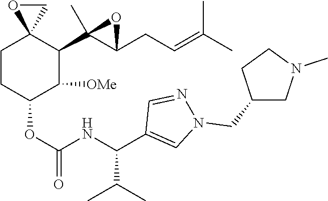 [SMILES: CO[C@@H]1[C@H](OC(=O)N[C@H](c2cnn(C[C@@H]3CCN(C)C3)c2)C(C)C)CC[C@]2(CO2)[C@H]1[C@@]1(C)O[C@@H]1CC=C(C)C] | 545 [M + H]⁺ | 7.26-7.29 (m, 2H), 5.36 (s, 1H), 5.13 (m, 1H), 4.99 (d, J = 8.0 Hz, 1H), 4.48 (m, J = 8.0 Hz 1H), 4.11 (d, J = 2.4 Hz, 1H), 3.58 (d, J = 10.4 Hz, 1H), 3.37 (s, 3H), 3.03-3.20 (m, 4H), 2.90 (d, J = 2.4 Hz, 1H), 2.64 (s, 3H), 2.46-2.52 (m, 2H), 2.27-2.31 (m, 1H), 2.08-2.15 (m, 2H), 1.88-1.97 (m, 3H), 1.74-1.81 (m, 2H), 1.67 (s, 3H), 1.58 (s, 3H), 1.14 (s, 3H), 0.97 (d, J = 8.4 Hz, 1H), 0.81-0.87 (m, 6H) |
| 24 | 25 | 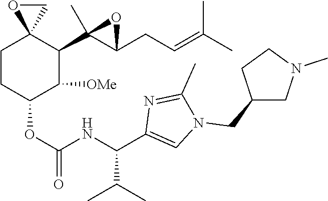 [SMILES: CO[C@@H]1[C@H](OC(=O)N[C@H](c2cn(C[C@H]3CCN(C)C3)c(C)n2)C(C)C)CC[C@]2(CO2)[C@H]1[C@@]1(C)O[C@@H]1CC=C(C)C] | 559 [M + H]⁺ | 6.74-6.68 (m, 1H), 6.49 (d, J = 9.6 Hz, 1H), 5.44 (s, 1H), 5.12 (t, J = 7.4 Hz, 1H), 4.38-4.29 (m, 1H), 4.02 (dd, J = 14.1, 8.4 Hz, 1H), 3.88 (dd, J = 14.2, 6.9 Hz, 1H), 3.53 (dd, J = 11.3, 2.6 Hz, 1H), 3.43 (t, J = 7.9 Hz, 1H), 3.33 (s, 3H), 3.22-3.10 (m, 1H), 2.99-2.91 (m, 1H), 2.90-2.79 (m, 3H), 2.65 (d, J = 6.2 Hz, 3H), 2.56-2.36 (m, 5H), 2.33-2.22 (m, 1H), 2.20-1.97 (m, 4H), 1.88 (d, J = 11.3 Hz, 2H), 1.83-1.69 (m, 2H), 1.66 (d, J = 9.7 Hz, 3H), 1.58 (s, 3H), |
| | | | | 1.11 (s, 3H), 0.98 (d, J = 11.9 Hz, |
| | | | | 1H), 0.88 (d, J = 6.7 Hz, 3H), 0.74 |
| | | | | (d, J = 6.7 Hz, 3H) |
| 25 | 26 | 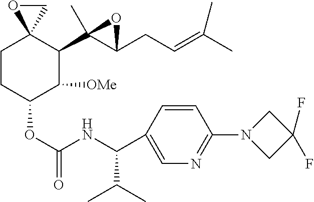 [SMILES: CO[C@@H]1[C@H](OC(=O)N[C@H](c2ccc(N3CC(F)(F)C3)nc2)C(C)C)CC[C@]2(CO2)[C@H]1[C@@]1(C)O[C@@H]1CC=C(C)C] | 550 [M + H]⁺ | 8.06 (s, 1H), 7.40 (m, 1H), 6.36 (d, J = 8.5 Hz, 1H), 5.37 (d, J = 24.1 Hz, 1H), 5.19 (m, 2H), 4.34 (t, J = 12.0 Hz, 4H), 3.61 (m, J = 8.6 Hz, 1H), 3.38 (d, J = 39.9 Hz, 3H), 2.95 (d, J = 4.2 Hz, 1H), 2.68-2.45 (m, 2H), 2.37 (m, 1H), 2.22-1.89 (m, 4H), 1.74 (s, 3H), 1.66 (s, 7H), 1.19 (s, 3H), 0.96 (m, 3H), 0.83 (m, 3H) |
| 26 | 27 | 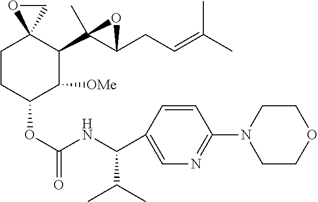 [SMILES: CO[C@@H]1[C@H](OC(=O)N[C@H](c2ccc(N3CCOCC3)nc2)C(C)C)CC[C@]2(CO2)[C@H]1[C@@]1(C)O[C@@H]1CC=C(C)C] | 544 [M + H]⁺ | 8.07 (s, 1H), 7.38 (dd, J = 8.7, 2.5 Hz, 1H), 6.60 (d, J = 8.7 Hz, 1H), 5.48-4.87 (m, 3H), 4.33 (d, J = 6.8 Hz, 1H), 3.94-3.76 (m, 4H), 3.61 (d, J = 10.5 Hz, 1H), 3.40 (d, J = 54.3 Hz, 6H), 2.95 (s, 1H), 2.56 (d, J = 16.9 Hz, 1H), 2.36 (m, 1H), 2.22- 1.88 (m, 4H), 1.73 (d, J = 8.2 Hz, 7H), 1.65 (s, 3H), 1.20 (s, 3H), 0.94 (d, J = 6.6 Hz, 3H), 0.85 (d, J = 9.7 Hz, 3H) |
| 27 | 28 | 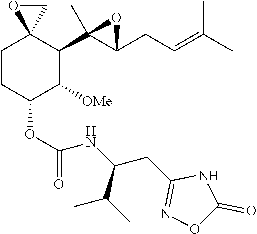 [SMILES: CO[C@@H]1[C@H](OC(=O)N[C@@H](Cc2noc(=O)[nH]2)C(C)C)CC[C@]2(CO2)[C@H]1[C@@]1(C)O[C@@H]1CC=C(C)C] | 480 [M + H]⁺ | 10.15 (m, 1H), 5.45 (m, 1H), 5.14 (m, 1H), 4.64 (m, 1H), 3.63 (s, 2H), 3.37 (s, 3H), 2.91 (s, 1H), 2.77-2.49 (m, 4H), 2.08 (m, 1H), 1.99-1.72 (m, 6H), 1.69 (s, 3H), 1.60 (s, 3H), 1.17 (d, J = 8.0 Hz, 3H), 1.03 (s, 1H), 0.91 (m, 6H) |
| 28 | 29 | 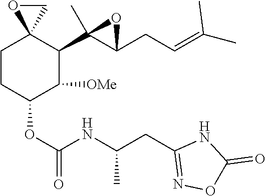 [SMILES: CO[C@@H]1[C@H](OC(=O)N[C@@H](C)Cc2noc(=O)[nH]2)CC[C@]2(CO2)[C@H]1[C@@]1(C)O[C@@H]1CC=C(C)C] | 452 [M + H]⁺ | 10.68 (s, 1H), 5.72 (s, 1H), 5.52 (s, 1H), 5.10 (t, J = 7.3 Hz, 1H), 4.08 (s, 1H), 3.63 (dd, J = 11.4, 2.3 Hz, 1H), 3.42 (s, 3H), 2.94-2.80 (m, 3H), 2.52 (dd, J = 26.6, 8.7 Hz, 2H), 2.31 (d, J = 10.2 Hz, 2H), 2.08-1.87 (m, 3H), 1.80 (d, J = 14.7 Hz, 1H), 1.70 (d, J = 4.7 Hz, 3H), 1.58 (s, 3H), 1.19- 1.04 (m, 6H), 0.97 (d, J = 12.2 Hz, 1H) |
| 29 | 30 | 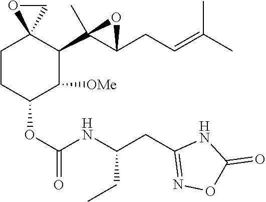 [SMILES: CC[C@@H](Cc1noc(=O)[nH]1)NC(=O)O[C@@H]1CC[C@]2(CO2)[C@@H]([C@@]2(C)O[C@@H]2CC=C(C)C)[C@@H]1OC] | 466 [M + H]⁺ | 5.51 (s, 1H), 5.39 (s, 1H), 5.11 (t, J = 7.5 Hz, 1H), 3.80 (s, 1H), 3.62 (d, J = 9.3 Hz, 1H), 3.42 (s, 3H), 2.96-2.74 (m, 2H), 2.60 (d, J = 10.3 Hz, 1H), 2.49 (d, J = 4.1 Hz, 1H), 2.46-2.40 (m, 1H), 2.39-2.32 (m, 1H), 1.99 (ddd, J = 32.8, 20.3, 11.2 Hz, 4H), 1.78 (t, J = 15.8 Hz, 2H), 1.69 (s, 3H), 1.59 (s, 3H), 1.53-1.37 (m, 2H), 1.18 (s, 1H), 1.10 (s, 3H), 1.02- 0.89 (m, 4H) |
| 30 | 31 | 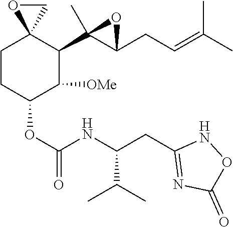 [SMILES: CO[C@@H]1[C@H](OC(=O)N[C@H](Cc2nc(=O)o[nH]2)C(C)C)CC[C@]2(CO2)[C@H]1[C@@]1(C)O[C@@H]1CC=C(C)C] | 480 [M + H]⁺ | 5.62 (d, J = 9.2 Hz, 1H), 5.45 (s, 1H), 5.19 (t, J = 7.6 Hz, 1H), 3.68-3.70 (m, 2H), 3.49 (s, 3H), 2.95 (d, J = 4.0 Hz, 1H), 2.71-2.85 (m, 2H), 2.55 (d, J = 3.6 Hz, 1H), 2.34-2.46 (m, 2H), 2.03-2.14 (m, 3H), 1.99-2.01 (m, 2H), 1.78 (s, 3H), 1.64 (s, 4H), 1.21 (s, 3H), 1.04-1.15 (m, 7H) |
| 31 | 32 | 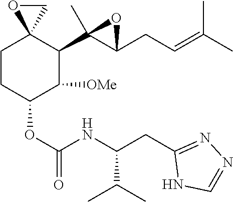 [SMILES: CO[C@@H]1[C@H](OC(=O)N[C@H](Cc2nnc[nH]2)C(C)C)CC[C@]2(CO2)[C@H]1[C@@]1(C)O[C@@H]1CC=C(C)C] | 463 [M + H]⁺ | 7.98 (s, 1H), 5.44 (s, 1H), 5.19-5.23 (m, 1H), 4.76 (d, J = 9.6 Hz, 1H), 3.73 (d, J = 5.2 Hz, 1H), 3.58 (d, J = 11.6 Hz, 1H), 3.34 (s, 1H), 3.06-3.10 (m, 1H), 2.98 (d, J = 4.0 Hz, 1H), 2.84-2.89 (m, 1H), 2.72-2.75 (m, 1H), 2.55 (d, J = 3.6 Hz, 1H), 2.43- 2.47 (m, 1H), 2.03-2.22 (m, 3H), 1.82-1.98 (m, 4H), 1.76 (s, 3H), 1.67 (s, 3H), 1.22 (s, 3H), 1.02-1.06 (m, 4H), 0.99 (d, J = 6.0 Hz, 3H) |
| 32 | 33 | 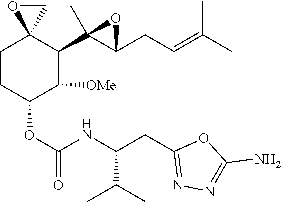 [SMILES: CO[C@@H]1[C@H](OC(=O)N[C@H](Cc2nnc(N)o2)C(C)C)CC[C@]2(CO2)[C@H]1[C@@]1(C)O[C@@H]1CC=C(C)C] | 479 [M + H]⁺ | 5.61-5.44 (m, 3H), 5.18 (t, 1H), 4.70 (d, J = 9.6 Hz, 1H), 3.81-3.70 (m, 1H), 3.61 (m, 1H), 3.15 (m, 1H), 2.97 (m, 1H), 2.73 (m, 1H), 2.64- 2.53 (m, 2H), 2.43-2.33 (m, 1H), 2.29-2.06 (m, 3H), 1.97-1.79 (m, 3H), 1.75 (s, 4H), 1.66 (s, 3H), 1.18 (s, 3H), 1.08 (m, 1H), 1.01 (m, 3H), 0.97 (d, J = 6.7 Hz, 3H) |
| 33 | 34 | 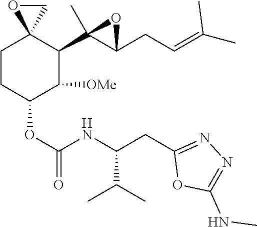 [SMILES: CNc1nnc(C[C@@H](NC(=O)O[C@@H]2CC[C@]3(CO3)[C@@H]([C@@]3(C)O[C@@H]3CC=C(C)C)[C@@H]2OC)C(C)C)o1] | 493 [M + H]⁺ | 6.06 (d, J = 4.9 Hz, 1H), 5.53 (d, J = 2.7 Hz, 1H), 5.13 (t, J = 7.5 Hz, 1H), 4.54 (d, J = 9.5 Hz, 1H), 3.65 (dd, J = 11.1, 7.5, 4.0 Hz, 1H), 3.54 (dd, J = 11.4, 2.7 Hz, 1H), 3.30 (s, 3H), 3.08 (dd, J = 15.0, 3.9 Hz, 1H), 2.89 (dd, J = 10.3, 4.6 Hz, 4H), 2.64 (dd, J = 15.0, 7.7 Hz, 1H), 2.57-2.47 (m, 2H), 2.35-2.24 (m, 1H), 2.14-1.73 (m, 6H), 1.70-1.62 (m, 4H), 1.59 (s, 3H), 1.13 (s, 3H), 0.99 (d, J = 13.7 Hz, 1H), 0.94 (d, J = 6.7 Hz, 3H), 0.88 (d, J = 6.7 Hz, 3H) |
| 34 | 35 | 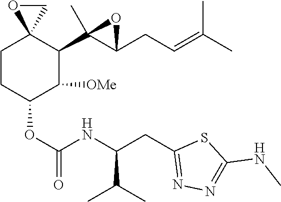 [SMILES: CNc1nnc(C[C@H](NC(=O)O[C@@H]2CC[C@]3(CO3)[C@@H]([C@@]3(C)O[C@@H]3CC=C(C)C)[C@@H]2OC)C(C)C)s1] | 509 [M + H]⁺ | 5.30 (s, 1H), 5.14 (t, 1H), 4.87 (d, J = 9.8 Hz, 1H), 3.78-3.62 (m, 1H), 3.57-3.53 (m, 1H), 3.32 (d, J = 19.9 Hz, 3H), 3.05-2.97 (m, 1H), 2.95 (s, 3H), 2.90 (d, J = 4.3 Hz, 1H), 2.55- 2.44 (m, 2H), 2.37-2.24 (m, 1H), 2.11-2.09 (m, 1H), 2.03-1.93 (m, 1H), 1.91-1.73 (m, 3H), 1.68 (s, 3H), 1.59 (s, 3H), 1.52 (s, 2H), 1.13 (s, 3H), 0.96 (s, 1H), 0.91 (m, 6H) |
| 35 | 36 | 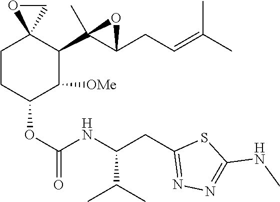 [SMILES: CNc1nnc(C[C@@H](NC(=O)O[C@@H]2CC[C@]3(CO3)[C@@H]([C@@]3(C)O[C@@H]3CC=C(C)C)[C@@H]2OC)C(C)C)s1] | 509 [M + H]⁺ | 5.42 (s, 1H), 5.13 (t, 1H), 4.74 (d, J = 9.7 Hz, 1H), 3.68 (m, 1H), 3.52 (m, 1H), 3.32 (s, 3H), 3.15 (m, 1H), 2.96 (d, J = 9.9 Hz, 3H), 2.89 (d, J = 4.2 Hz, 1H), 2.80 (m, 1H), 2.54 (t, 1H), 2.49 (d, J = 4.2 Hz, 1H), 2.36-2.23 (m, 1H), 2.15-2.01 (m, 2H), 1.95- 1.87 (m, 2H), 1.86-1.71 (m, 3H), 1.68 (s, 3H), 1.59 (s, 3H), 1.13 (s, 3H), 1.00 (d, J = 13.8 Hz, 1H), 0.90 (m, 6H) |
| 36 | 37 | 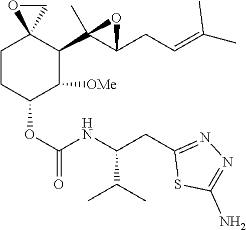 [SMILES: CO[C@@H]1[C@H](OC(=O)N[C@H](Cc2nnc(N)s2)C(C)C)CC[C@]2(CO2)[C@H]1[C@@]1(C)O[C@@H]1CC=C(C)C] | 495 [M + H]⁺ | 5.67 (s, 1H), 5.42 (s, 1H), 5.12 (t, J = 7.4 Hz, 1H), 4.70 (d, J = 9.9 Hz, 1H), 3.78-3.62 (m, 1H), 3.50 (dd, J = 11.4, 2.4 Hz, 1H), 3.30 (d, J = 18.6 Hz, 3H), 3.24-3.15 (m, 1H), 2.90 (d, J = 4.2 Hz, 1H), 2.73 (dd, J = 14.8, 9.7 Hz, 1H), 2.55 (t, J = 6.4 Hz, 1H), 2.49 (d, J = 4.2 Hz, 1H), 2.35- 2.27 (m, 1H), 2.16-2.01 (m, 2H), 1.91 (t, J = 14.0 Hz, 2H), 1.81-1.72 (m, 2H), 1.68 (s, 3H), 1.59 (s, 3H), 1.12 (s, 3H), 1.04-0.85 (m, 7H) |
| 37 | 38 | 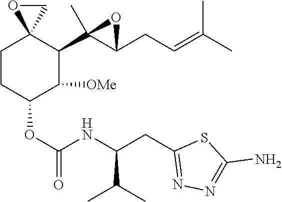 [SMILES: CO[C@@H]1[C@H](OC(=O)N[C@@H](Cc2nnc(N)s2)C(C)C)CC[C@]2(CO2)[C@H]1[C@@]1(C)O[C@@H]1CC=C(C)C] | 495 [M + H]⁺ | 5.43 (s, 3H), 5.12 (s, 1H), 4.60-4.62 (d, J = 9.6 Hz, 1H), 3.69 (s, 1H), 3.48-3.51 (d, J = 11.2 Hz, 1H), 3.27 (s, 3H), 3.19-3.22 (d, J = 15.2 Hz, 1H), 2.90-2.91 (d, J = 2.4 Hz, 1H), 2.69-2.75 (m, 1H), 2.48-2.54 (m, 2H), 2.29-2.32 (m, 1H), 2.03-2.09 (m, 2H), 1.86-1.96 (m, 2H), 1.73- 1.80 (m, 2H), 1.68 (s, 3H), 1.58 (s, 3H), 1.12 (s, 3H), 0.88-1.01 (m, 7H) |
| 38 | 39 | 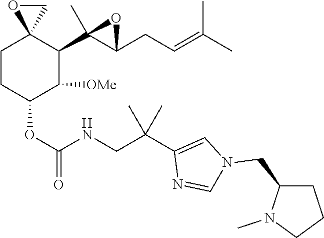 [SMILES: CO[C@@H]1[C@H](OC(=O)NCC(C)(C)c2cn(C[C@H]3CCCN3C)cn2)CC[C@]2(CO2)[C@H]1[C@@]1(C)O[C@@H]1CC=C(C)C] | 545 [M + H]⁺ | 8.06 (s, 1H), 6.91 (s, 1H), 5.53-5.56 (m, 1H), 5.43 (s, 1H), 5.18-5.22 (m, 1H), 4.49-4.54 (m, 1H), 4.18-4.23 (m, 1H), 3.39-3.64 (m, 2H), 3.37- 3.43 (m, 4H), 3.27-3.31 (m, 1H), 3.15-3.22 (m, 1H), 2.96 (d, J = 3.6 Hz, 1H), 2.69-2.76 (m, 1H), 2.59- 2.62 (m, 1H), 2.56 (d, J = 4.4 Hz, 1H), 2.46 (s, 3H), 2.30-2.37 (m, 1H), 2.09-2.22 (m, 3H), 1.81-2.06 (m, 6H), 1.75 (s, 3H), 1.66 (s, 3H), 1.31 (s, 6H), 1.20 (s, 3H), 1.03 (d, J = 13.2 Hz, 1H) |
| 39 | 40 | 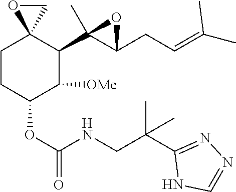 [SMILES: CO[C@@H]1[C@H](OC(=O)NCC(C)(C)c2nnc[nH]2)CC[C@]2(CO2)[C@H]1[C@@]1(C)O[C@@H]1CC=C(C)C] | 449 [M + H]⁺ | 8.01 (s, 1H), 5.50 (s, 1H), 5.24 (d, J = 7.3 Hz, 2H), 3.67 (dd, J = 11.3, 2.4 Hz, 1H), 3.60-3.42 (m, 4H), 3.39- 3.30 (m, 1H), 2.98 (d, J = 4.2 Hz, 1H), 2.68 (t, J = 6.4 Hz, 1H), 2.56 (d, J = 4.2 Hz, 1H), 2.48-2.37 (m, 1H), 2.21-1.83 (m, 5H), 1.77 (s, 3H), 1.68 (s, 3H), 1.43 (s, 3H), 1.26-1.20 (m, 3H), 1.05 (d, J = 14.2 Hz, 1H) |
| 40 | 41 | 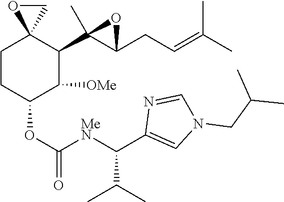 [SMILES: CO[C@@H]1[C@H](OC(=O)N(C)[C@H](c2cn(CC(C)C)cn2)C(C)C)CC[C@]2(CO2)[C@H]1[C@@]1(C)O[C@@H]1CC=C(C)C] | 518 [M + H]⁺ | 7.28 (d, J = 8.3 Hz, 1H), 6.69 (d, J = 35.8 Hz, 1H), 5.50 (d, J = 2.2 Hz, 1H), 5.12 (t, J = 6.2 Hz, 1H), 4.68 (dd, J = 58.9, 10.9 Hz, 1H), 3.66- 3.52 (m, 3H), 3.35 (s, 3H), 2.91 (d, J = 4.3 Hz, 1H), 2.76 (d, J = 11.8 Hz, 3H), 2.52-2.20 (m, 4H), 2.10-1.86 (m, 4H), 1.78 (t, J = 10.9 Hz, 2H), 1.66 (s, 3H), 1.58 (d, J = 4.9 Hz, 3H), 1.20-1.11 (m, 4H), 0.99 (dd, J = 28.5, 9.8 Hz, 2H), 0.87 (d, J = 6.6 Hz, 2H), 0.84 (dd, J = 6.5, 4.3 Hz, 5H), 0.79 (d, J = 6.2 Hz, 1H), 0.74 |
| | | | | (d, J = 6.5 Hz, 2H) |

Following the above procedures, the following compounds are prepared:

| Example | |
|---|---|
| No. | Structure |
| 41 | 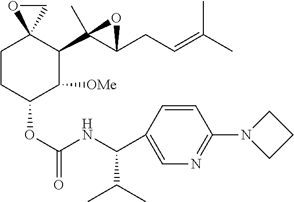 [SMILES: CO[C@@H]1[C@H](OC(=O)N[C@H](c2ccc(N3CCC3)nc2)C(C)C)CC[C@]2(CO2)[C@H]1[C@@]1(C)O[C@@H]1CC=C(C)C] |
| 42 | 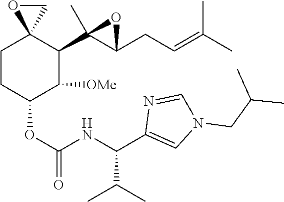 [SMILES: CO[C@@H]1[C@H](OC(=O)N[C@H](c2cn(CC(C)C)cn2)C(C)C)CC[C@]2(CO2)[C@H]1[C@@]1(C)O[C@@H]1CC=C(C)C] |

### Biological Example A

Compounds are tested for their capacity to inhibit recombinant human MetAP2 activity using the following assay.

Flag tagged Human recombinant MetAP2 expressed and isolated for use as the enzyme source. 10 mM stock solutions of compounds were prepared in 100% DMSO and further diluted in 100% DMSO required concentration to 1 mM stocks. The stock compound solutions and DMSO vehicle controls were diluted to target final compound concentrations using assay buffer to a final concentration of 50 mM HEPES containing 100 mM NaCl, pH adjusted to 7.5. The MAS peptide was formulated to a 7.5 mM stock in distilled water and prior to use further diluted 1:4. Amino acid oxidase was prepared as a stock solution (6.2 mg/ml) and prior to use further diluted 1:49.6 in distilled water. A 250 μM solution of MnCl₂ was prepared in advance of thawing an aliquot of MetAP2 enzyme. 40 μl of enzyme was mixed with 100 μl of MnCl₂ then further diluted in assay buffer to a final concentration of 16 μg/ml. To test for compound effect on MetAP2 enzyme activity, 5 μl of test compound, 10 μl of MAS substrate/amino acid oxidase mixture, 10 μl of MetAP2 was added to test wells in a 384 well black plate with blank wells containing no enzyme, replaced with 10 μl of assay buffer. All compounds were tested in duplicate on two occasions on the same day. The final in well concentrations of the assay were: 1% DMSO, 0.272 μg/ml MetAP2, 10 μM MnCl₂, 50.0 μg/ml (0.225 U/ml) amino acid oxidase, and 0.075 mM MAS.

The plate was sealed with a TopSeal A cover and mixed briefly on an orbital mixer at 900 rpm. The plate was incubated for a further 25 minutes at 25° C. A 5× stock of Amplex buffer was prepared (0.25 M sodium phosphate, pH 7.4) and stored at 4° C. When preparing for use the stock was diluted with distilled water Amplex Ultraread stock solution was prepared at 2.57 mg/ml in 100% DMSO and stored in 50 μl aliquots at −20° C. 20 μl of 505 U/ml. Horse radish peroxidase was diluted in 990 ml of Amplex buffer, 100 μl of this was combined with 50 μl of Amplex Ultrared in 4850 ml of 1× Amplex buffer to generate sufficient detection reagent for a 384 well plate. 25 μl detection reagent was added to each well of the test plate, which was re-sealed and mixed briefly on an orbital shaker. The plate was transferred to an Envision Multi-label reader and RFU measured corresponding to excitation 531 nm and emission 595 nm. At the end of the MetAP2 incubation 25 μl Amplex/HRP mixture per well was added and the plate read plate on a plate reader.

The IC₅₀ is defined as the concentration at which a given compound achieves 50% inhibition of control.

Compounds of the disclosure demonstrated activity in the assay of this Example as indicated in the following table, wherein A represents an IC₅₀ of <0.05 μM and B represents an IC₅₀ between 0.05 μM and 0.5 μM.

TABLE 1

| | | MetAP2 |
|---|---|---|
| | | IC₅₀ |
| Example | Compound Name | (μM) |
| 1 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-1-(5-amino-1,3,4-oxadiazol- | |
| | 2-yl)-2-methylpropylcarbamate | |
| 4 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-1-(1H-imidazol-2-yl)-2- | |
| | methylpropylcarbamate | |
| 5 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((R)-1- | |
| | methylpyrrolidin-2-yl)methyl)-1H-imidazol-2-yl)propylcarbamate formate | |
| 6 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((S)-1- | |
| | methylpyrrolidin-2-yl)methyl)-1H-imidazol-2-yl)propylcarbamate formate | |
| 7 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (R)-2-methyl-1-(5- | |
| | (methylamino)-1,3,4-thiadiazol-2-yl)propylcarbamate | |
| 8 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(5- | |
| | (methylamino)-1,3,4-thiadiazol-2-yl)propylcarbamate | |
| 16 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((R)-1- | |
| | methylpyrrolidin-2-yl)methyl)-1H-imidazol-4-yl)propylcarbamate | |
| 9 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-2-methyl-1-(4H-1,2,4-triazol- | |
| | 3-yl)propylcarbamate | |
| 10 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(5-oxo-4,5- | |
| | dihydro-1,2,4-oxadiazol-3-yl)propylcarbamate | |
| 11 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (R)-2-methyl-1-(5-oxo-4,5- | |
| | dihydro-1,2,4-oxadiazol-3-yl)propylcarbamate | |
| 2 | (3R,5S,6R)-5-methoxy-4-((2R,3R)-2-methyl-3-(3-methylbut-2-enyl)oxiran-2- | A |
| | yl)-1-oxaspiro[2.5]octan-6-yl (S)-2-methyl-1-(5-(methylamino)-1,3,4- | |
| | oxadiazol-2-yl)propylcarbamate | |
| 3 | (3R,5S,6R)-5-methoxy-4-((2R,3R)-2-methyl-3-(3-methylbut-2-enyl)oxiran-2- | B |
| | yl)-1-oxaspiro[2.5]octan-6-yl (R)-2-methyl-1-(5-(methylamino)-1,3,4- | |
| | oxadiazol-2-yl)propylcarbamate | |
| 12 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (R)-2-methyl-1-(1-(((S)-1- | |
| | methylpyrrolidin-3-yl)methyl)-1H-imidazol-4-yl)propylcarbamate formate | |
| 13 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((R)-1- | |
| | methylpyrrolidin-3-yl)methyl)-1H-imidazol-4-yl)propylcarbamate | |
| 20 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-2-methyl-1-(4-oxo-1,4- | |
| | dihydropyridin-2-yl)propylcarbamate | |
| 21 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-2-methyl-1-(1H-1,2,3-triazol- | |
| | 4-yl)propylcarbamate | |
| 14 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-2-methyl-1-(1-(((S)-1- | |
| | methylpyrrolidin-2-yl)methyl)-1H-imidazol-4-yl)propylcarbamate formate | |
| 15 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-2-methyl-1-(1-(((R)-1- | |
| | methylpyrrolidin-3-yl)methyl)-1H-imidazol-4-yl)propylcarbamate formate | |
| 17 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(2- | |
| | morpholinoethyl)-1H-imidazol-4-yl)propylcarbamate | |
| 22 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((S)-1- | |
| | methylpyrrolidin-3-yl)methyl)-1H-pyrazol-4-yl)propylcarbamate formate | |
| 24 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(2-methyl-1-(((S)- | |
| | 1-methylpyrrolidin-3-yl)methyl)-1H-imidazol-4-yl)propylcarbamate | |
| 27 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-3-methyl-1-(5-oxo-4,5- | |
| | dihydro-1,2,4-oxadiazol-3-yl)butan-2-ylcarbamate | |
| 31 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (R)-3-methyl-1-(4H-1,2,4-triazol- | |
| | 3-yl)butan-2-ylcarbamate | |
| 32 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (R)-1-(5-amino-1,3,4-oxadiazol- | |
| | 2-yl)-3-methylbutan-2-ylcarbamate | |
| 38 | (S)-((3R,4S,5S,6R)-5-methoxy-4-((2R,3R)-2-methyl-3-(3-methylbut-2- | B |
| | enyl)oxiran-2-yl)-1-oxaspiro[2.5]octan-6-yl)2- | |
| | ((diethylamino)methyl)pyrrolidine-1-carboxylate formate | |
| 34 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-3-methyl-1-(5- | |
| | (methylamino)-1,3,4-thiadiazol-2-yl)butan-2-ylcarbamate | |
| 35 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (R)-3-methyl-1-(5- | |
| | (methylamino)-1,3,4-thiadiazol-2-yl)butan-2-ylcarbamate | |
| 33 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (R)-3-methyl-1-(5- | |
| | (methylamino)-1,3,4-oxadiazol-2-yl)butan-2-ylcarbamate | |
| 39 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl 2-methyl-2-(4H-1,2,4-triazol-3- | |
| | yl)propylcarbamate | |
| 28 | (3R,4S,5S,6R)-5-methoxy-4-((2R,3R)-2-methyl-3-(3-methylbut-2-en-1- | A |
| | yl)oxiran-2-yl)-1-oxaspiro[2.5]octan-6-yl ((S)-1-(5-oxo-4,5-dihydro-1,2,4- | |
| | oxadiazol-3-yl)propan-2-yl)carbamate | |
| 29 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl (S)-1-(5-oxo-4,5-dihydro-1,2,4- | |
| | oxadiazol-3-yl)butan-2-ylcarbamate | |
| 36 | (R)-((3R,4S,5S,6R)-5-methoxy-4-((2R,3R)-2-methyl-3-(3-methylbut-2- | B |
| | enyl)oxiran-2-yl)-1-oxaspiro[2.5]octan-6-yl) 2- | |
| | ((ethylamino)methyl)azetidine-1-carboxylate formate | |
| 30 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (R)-3-methyl-1-(5-oxo-4,5- | |
| | dihydro-1,2,4-oxadiazol-3-yl)butan-2-ylcarbamate | |
| 37 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-1-(5-amino-1,3,4-thiadiazol- | |
| | 2-yl)-3-methylbutan-2-ylcarbamate | |
| 18 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-1-(1-(((S)-1-(2,2- | |
| | difluoroethyl)pyrrolidin-3-yl)methyl)-1H-imidazol-4-yl)-2- | |
| | methylpropylcarbamate | |
| 23 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(1-(((R)-1- | |
| | methylpyrrolidin-3-yl)methyl)-1H-pyrazol-4-yl)propylcarbamate | |
| 25 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-1-(6-(3,3-difluoroazetidin-1- | |
| | yl)pyridin-3-yl)-2-methylpropylcarbamate | |
| 26 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl (S)-2-methyl-1-(6- | |
| | morpholinopyridin-3-yl)propylcarbamate | |
| 19 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl ((S)-1-(l-(((R)-1-(2,2- | |
| | difluoroethyl)pyrrolidin-3-yl)methyl)-1H-imidazol-4-yl)-2- | |
| | methylpropyl)carbamate | |
| 40 | (3R,4S,5S,6R)-5-methoxy-4-((2R,3R)-2-methyl-3-(3-methylbut-2- | B |
| | enyl)oxiran-2-yl)-1-oxaspiro[2.5]octan-6-yl ((S)-1-(1-isobutyl-1H-imidazol-4- | |
| | yl)-2-methylpropyl)(methyl)carbamate | |
| 41 | (3R,4S,5S,6R)-5-methoxy-4-((2R,3R)-2-methyl-3-(3-methylbut-2- | A |
| | enyl)oxiran-2-yl)-1-oxaspiro[2.5]octan-6-yl ((S)-1-(6-(azetidin-1-yl)pyridin- | |
| | 3-yl)-2-methylpropyl)carbamate | |
| 42 | (3R,4S,5S,6R)-5-methoxy-4-((2R,3R)-2-methyl-3-(3-methylbut-2-en-1- | A |
| | yl)oxiran-2-yl)-1-oxaspiro[2.5]octan-6-yl ((S)-1-(1-isobutyl-1H-imidazol-4- | |
| | yl)-2-methylpropyl)carbamate | |
| compound | [(3R,6R,7S,8S)-7-methoxy-8-[(2R,3R)-2-methyl-3-(3-methylbut-2- | B |
| A | enyl)oxiran-2-yl]-2-oxaspiro[2.5]octan-6-yl] (E)-3-[4-[2- | |
| | (dimethylamino)ethoxy]phenyl]prop-2-enoate | |

### Biological Example B

### Study Summary Mouse DIO:

The objective of this study design was to investigate the efficacy of disclosed compounds in a 10 day DIO mouse model. Effects on body weight, food intake, hematology and blood chemistry were the primary readouts of this study design. Male, Diet Induced Obese (DIO) C57BL/6 mice, 19-21 weeks of age (13-15 weeks on high fat diet) were ordered from a certified vendor and upon receipt were housed on irradiated corncob bedding in micro-isolator cages on a 12-hour light-dark cycle (0700-1900) at 68-74° F. and 30-70% humidity. Mice were fed Research Diets D12492 (60% Kcal fat, 20% Kcal carbohydrate and 20% protein) and provided water ad libitum. DIO mice were received and housed in the facility for approximately two weeks prior to the start of test article administration. On Day −4 or −3, mice were randomized into study groups based on body weight and body weights were continued to be recorded daily for the duration of the study. Daily food intake was assessed starting on Study Day −2 by weighing of the food with hopper together to avoid loss of food in transfer.

Compounds were formulated into a 100% DMSO stock (at 9 mg/mL) prior to the start, and further diluted into the target working concentration using the vehicle of 10% DMSO in water. Prior to test article administration, starting on Day −3 a dosing acclimation occurred with all animals receiving a subcutaneous injection of vehicle (10% DMSO) only for 3 days. Starting on Day 1, test compounds or vehicle were administered based on individual body weight, subcutaneously, once a day for 10 days. All mice were sacrificed on Day 11, 24 hours following the final dosing on Day 10. After sacrifice, whole blood was collected and analyzed for hematology and blood chemistry parameters.

### Study Summary Rat DIO:

The objective of this study design is to investigate the efficacy of disclosed compounds in an 11 day rat DIO model used to screen compounds for pharmacologic efficacy on endpoints related to obesity and metabolism. Effects on body weight, food intake, hematology and blood chemistries were the primary readouts of this study design. Male Sprague Dawley rats, approximately 8 weeks of age, were ordered from a certified vendor and housed on irradiated corncob bedding in micro-isolator cages, on a 12-hour light-dark cycle (0700-1900) at 68-74° F. and 30-70% humidity. Rats were fed Research Diets D12451 (45% High Fat) and provided water ad libitum. Rats were received and housed in the facility for at least two or three weeks prior to start of test article administration. On Day −4 or −3, rats were randomized into study groups based on body weight and body weights were continued to be recorded daily for the duration of the study. Daily food intake was assessed starting on Study Day −2 by weighing the hopper including the food to avoid loss of food in transfer.

Compounds were formulated into a 100% DMSO stock (at 9 mg/mL) prior to the start, and further diluted into the target working concentration using the vehicle of 10% DMSO in water. Prior to test article administration, starting on Day −3 a dosing acclimation occurred with all animals receiving a subcutaneous injection of vehicle (10% DMSO) only for 3 days. Starting on Day 1, test compounds or vehicle were administered based on individual body weight, subcutaneously, once a day for 11 days. All animals were sacrificed on Day 11, approximately 2 hours following the final dosing on Day 11. After sacrifice, whole blood was collected and analyzed for hematology and blood chemistry parameters.

Compounds were tested for weight loss vs. vehicle at 0.3 and 1.0 mpk sc (mg per kg animal weight delivered subcutaneously), and the results are shown in Table 2 below.

TABLE 2

| | | Mouse | Mouse | Rat | Rat |
|---|---|---|---|---|---|
| | | @ 1.0 | @ 0.3 | @ 0.3 | @ 1.0 |
| Example | | mpk sc | mpk sc | mpk sc | mpk sc |
| No. | Compound Name | (%) | (%) | (%) | (%) |
| 1 | (3R,4S,5S,6R)-5-methoxy-4-((2R,3R)- | 2.1 | 2.6 | | |
| | 2-methyl-3-(3-methylbut-2-enyl)oxiran- | | | | |
| | 2-yl)-1-oxaspiro[2.5]octan-6-yl (S)-1-(5- | | | | |
| | amino-1,3,4-oxadiazol-2-yl)-2- | | | | |
| | methylpropylcarbamate | | | | |
| 4 | (3R,4S,5S,6R)-5-methoxy-4-((2R,3R)- | 0.7 | 3.9 | | |
| | 2-methyl-3-(3-methylbut-2-enyl)oxiran- | | | | |
| | 2-yl)-1-oxaspiro[2.5]octan-6-yl (S)-1- | | | | |
| | (1H-imidazol-2-yl)-2- | | | | |
| | methylpropylcarbamate | | | | |
| 5 | (3R,4S,5S,6R)-5-methoxy-4-((2R,3R)- | | 3.1 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((R)-1-methylpyrrolidin-2- | | | | |
| | yl)methyl)-1H-imidazol-2- | | | | |
| | yl)propylcarbamate formate | | | | |
| 6 | (3R,4S,5S,6R)-5-methoxy-4-((2R,3R)- | | 6.5 | 0.1 | 2.1 |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((S)-1-methylpyrrolidin-2- | | | | |
| | yl)methyl)-1H-imidazol-2- | | | | |
| | yl)propylcarbamate formate | | | | |
| 7 | (3R,4S,5S,6R)-5-methoxy-4-((2R,3R)- | | 1.8 | | |
| | 2-methyl-3-(3-methylbut-2-enyl)oxiran- | | | | |
| | 2-yl)-1-oxaspiro[2.5]octan-6-yl (R)-2- | | | | |
| | methyl-1-(5-(methylamino)-1,3,4- | | | | |
| | thiadiazol-2-yl)propylcarbamate | | | | |
| 8 | (3R,4S,5S,6R)-5-methoxy-4-((2R,3R)- | | 2 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(5-(methylamino)-1,3,4- | | | | |
| | thiadiazol-2-yl)propylcarbamate | | | | |
| 16 | (3R,4S,5S,6R)-5-methoxy-4-((2R,3R)- | | 6.6 | 2.8 | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((R)-1-methylpyrrolidin-2- | | | | |
| | yl)methyl)-1H-imidazol-4- | | | | |
| | yl)propylcarbamate | | | | |
| 9 | (3R,4S,5S,6R)-5-methoxy-4-((2R,3R)- | | 2.1 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(4H-1,2,4-triazol-3- | | | | |
| | yl)propylcarbamate | | | | |
| 10 | (3R,4S,5S,6R)-5-methoxy-4-((2R,3R)- | 2.9 | 4.1 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(5-oxo-4,5-dihydro-1,2,4- | | | | |
| | oxadiazol-3-yl)propylcarbamate | | | | |
| 11 | (3R,4S,5S,6R)-5-methoxy-4-((2R,3R)- | 6.6 | −1.3 | | |
| | 2-methyl-3-(3-methylbut-2-enyl)oxiran- | | | | |
| | 2-yl)-1-oxaspiro[2.5]octan-6-yl (R)-2- | | | | |
| | methyl-1-(5-oxo-4,5-dihydro-1,2,4- | | | | |
| | oxadiazol-3-yl)propylcarbamate | | | | |
| 2 | (3R,5S,6R)-5-methoxy-4-((2R,3R)-2- | 4.6 | 5.4 | 0.6 | 0.9 |
| | methyl-3-(3-methylbut-2-enyl)oxiran-2- | | | | |
| | yl)-1-oxaspiro[2.5]octan-6-yl (S)-2- | | | | |
| | methyl-1-(5-(methylamino)-1,3,4- | | | | |
| | oxadiazol-2-yl)propylcarbamate | | | | |
| 3 | (3R,5S,6R)-5-methoxy-4-((2R,3R)-2- | | 1.1 | | |
| | methyl-3-(3-methylbut-2-enyl)oxiran-2- | | | | |
| | yl)-1-oxaspiro[2.5]octan-6-yl (R)-2- | | | | |
| | methyl-1-(5-(methylamino)-1,3,4- | | | | |
| | oxadiazol-2-yl)propylcarbamate | | | | |
| 12 | (3R,4S,5S,6R)-5-methoxy-4-((2R,3R)- | | 9.5 | 12.9 | 18.8 |
| | 2-methyl-3-(3-methylbut-2-enyl)oxiran- | | | | |
| | 2-yl)-1-oxaspiro[2.5]octan-6-yl (R)-2- | | | | |
| | methyl-1-(1-(((S)-1-methylpyrrolidin-3- | | | | |
| | yl)methyl)-1H-imidazol-4- | | | | |
| | yl)propylcarbamate formate | | | | |
| 13 | (3R,4S,5S,6R)-5-methoxy-4-((2R,3R)- | | 8.2 | 11.5 | 22 |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((R)-1-methylpyrrolidin-3- | | | | |
| | yl)methyl)-1H-imidazol-4- | | | | |
| | yl)propylcarbamate | | | | |
| 20 | (3R,4S,5S,6R)-5-methoxy-4-((2R,3R)- | 3.3 | 3.4 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(4-oxo-1,4-dihydropyridin-2- | | | | |
| | yl)propylcarbamate | | | | |
| 14 | (3R,4S,5S,6R)-5-methoxy-4-((2R,3R)- | 10.3 | 5.8 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((S)-1-methylpyrrolidin-2- | | | | |
| | yl)methyl)-1H-imidazol-4- | | | | |
| | yl)propylcarbamate formate | | | | |
| 15 | (3R,4S,5S,6R)-5-methoxy-4-((2R,3R)- | 8.4 | 10.8 | 10.6 | 16.4 |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((R)-1-methylpyrrolidin-3- | | | | |
| | yl)methyl)-1H-imidazol-4- | | | | |
| | yl)propylcarbamate formate | | | | |
| 17 | (3R,4S,5S,6R)-5-methoxy-4-((2R,3R)- | | 3.2 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(2-morpholinoethyl)-1H- | | | | |
| | imidazol-4-yl)propylcarbamate | | | | |
| 22 | (3R,4S,5S,6R)-5-methoxy-4-((2R,3R)- | | 4.7 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((S)-1-methylpyrrolidin-3- | | | | |
| | yl)methyl)-1H-pyrazol-4- | | | | |
| | yl)propylcarbamate formate | | | | |
| 24 | (3R,4S,5S,6R)-5-methoxy-4-((2R,3R)- | | 8.2 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(2-methyl-1-(((S)-1- | | | | |
| | methylpyrrolidin-3-yl)methyl)-1H- | | | | |
| | imidazol-4-yl)propylcarbamate | | | | |
| 27 | (3R,4S,5S,6R)-5-methoxy-4-((2R,3R)- | 5.4 | | −0.8 | 1.9 |
| | 2-methyl-3-(3-methylbut-2-enyl)oxiran- | | | | |
| | 2-yl)-1-oxaspiro[2.5]octan-6-yl (S)-3- | | | | |
| | methyl-1-(5-oxo-4,5-dihydro-1,2,4- | | | | |
| | oxadiazol-3-yl)butan-2-ylcarbamate | | | | |
| 31 | (3R,4S,5S,6R)-5-methoxy-4-((2R,3R)- | 2.5 | 2.4 | | |
| | 2-methyl-3-(3-methylbut-2-enyl)oxiran- | | | | |
| | 2-yl)-1-oxaspiro[2.5]octan-6-yl (R)-3- | | | | |
| | methyl-1-(4H-1,2,4-triazol-3-yl)butan-2- | | | | |
| | ylcarbamate | | | | |
| 32 | (3R,4S,5S,6R)-5-methoxy-4-((2R,3R)- | 0.8 | 3.9 | | |
| | 2-methyl-3-(3-methylbut-2-enyl)oxiran- | | | | |
| | 2-yl)-1-oxaspiro[2.5]octan-6-yl (R)-1- | | | | |
| | (5-amino-1,3,4-oxadiazol-2-yl)-3- | | | | |
| | methylbutan-2-ylcarbamate | | | | |
| 38 | (S)-((3R,4S,5S,6R)-5-methoxy-4- | 5.5 | 5 | −1.7 | |
| | ((2R,3R)-2-methyl-3-(3-methylbut-2- | | | | |
| | enyl)oxiran-2-yl)-1-oxaspiro[2.5]octan- | | | | |
| | 6-yl) 2- | | | | |
| | ((diethylamino)methyl)pyrrolidine-1- | | | | |
| | carboxylate formate | | | | |
| 34 | (3R,4S,5S,6R)-5-methoxy-4-((2R,3R)- | | 3.4 | | |
| | 2-methyl-3-(3-methylbut-2-enyl)oxiran- | | | | |
| | 2-yl)-1-oxaspiro[2.5]octan-6-yl (S)-3- | | | | |
| | methyl-1-(5-(methylamino)-1,3,4- | | | | |
| | thiadiazol-2-yl)butan-2-ylcarbamate | | | | |
| 35 | (3R,4S,5S,6R)-5-methoxy-4-((2R,3R)- | 1 | | | |
| | 2-methyl-3-(3-methylbut-2-enyl)oxiran- | | | | |
| | 2-yl)-1-oxaspiro[2.5]octan-6-yl (R)-3- | | | | |
| | methyl-1-(5-(methylamino)-1,3,4- | | | | |
| | thiadiazol-2-yl)butan-2-ylcarbamate | | | | |
| 33 | (3R,4S,5S,6R)-5-methoxy-4-((2R,3R)- | 1 | 3.7 | | |
| | 2-methyl-3-(3-methylbut-2-enyl)oxiran- | | | | |
| | 2-yl)-1-oxaspiro[2.5]octan-6-yl (R)-3- | | | | |
| | methyl-1-(5-(methylamino)-1,3,4- | | | | |
| | oxadiazol-2-yl)butan-2-ylcarbamate | | | | |
| 39 | (3R,4S,5S,6R)-5-methoxy-4-((2R,3R)- | 7.6 | 3.4 | | |
| | 2-methyl-3-(3-methylbut-2-enyl)oxiran- | | | | |
| | 2-yl)-1-oxaspiro[2.5]octan-6-yl 2- | | | | |
| | methyl-2-(4H-1,2,4-triazol-3- | | | | |
| | yl)propylcarbamate | | | | |
| 28 | (3R,4S,5S,6R)-5-methoxy-4-((2R,3R)- | 4.2 | −2.3 | −1.4 | |
| | 2-methyl-3-(3-methylbut-2-en-1- | | | | |
| | yl)oxiran-2-yl)-1-oxaspiro[2.5]octan-6- | | | | |
| | yl ((S)-1-(5-oxo-4,5-dihydro-1,2,4- | | | | |
| | oxadiazol-3-yl)propan-2-yl)carbamate | | | | |
| 29 | (3R,4S,5S,6R)-5-methoxy-4-((2R,3R)- | 3.6 | | | |
| | 2-methyl-3-(3-methylbut-2-enyl)oxiran- | | | | |
| | 2-yl)-1-oxaspiro[2.5]octan-6-yl (S)-1-(5- | | | | |
| | oxo-4,5-dihydro-1,2,4-oxadiazol-3- | | | | |
| | yl)butan-2-ylcarbamate | | | | |
| 36 | (R)-((3R,4S,5S,6R)-5-methoxy-4- | 2 | | | |
| | ((2R,3R)-2-methyl-3-(3-methylbut-2- | | | | |
| | enyl)oxiran-2-yl)-1-oxaspiro[2.5]octan- | | | | |
| | 6-yl) 2-((ethylamino)methyl)azetidine- | | | | |
| | 1-carboxylate formate | | | | |
| 30 | (3R,4S,5S,6R)-5-methoxy-4-((2R,3R)- | 14.1 | 1.4 | 0.7 | −0.4 |
| | 2-methyl-3-(3-methylbut-2-enyl)oxiran- | | | | |
| | 2-yl)-1-oxaspiro[2.5]octan-6-yl (R)-3- | | | | |
| | methyl-1-(5-oxo-4,5-dihydro-1,2,4- | | | | |
| | oxadiazol-3-yl)butan-2-ylcarbamate | | | | |
| 37 | (3R,4S,5S,6R)-5-methoxy-4-((2R,3R)- | | 2.5 | | |
| | 2-methyl-3-(3-methylbut-2-enyl)oxiran- | | | | |
| | 2-yl)-1-oxaspiro[2.5]octan-6-yl (S)-1-(5- | | | | |
| | amino-1,3,4-thiadiazol-2-yl)-3- | | | | |
| | methylbutan-2-ylcarbamate | | | | |
| 18 | (3R,4S,5S,6R)-5-methoxy-4-((2R,3R)- | | −0.4 | | |
| | 2-methyl-3-(3-methylbut-2-enyl)oxiran- | | | | |
| | 2-yl)-1-oxaspiro[2.5]octan-6-yl (S)-1-(1- | | | | |
| | (((S)-1-(2,2-difluoroethyl)pyrrolidin-3- | | | | |
| | yl)methyl)-1H-imidazol-4-yl)-2- | | | | |
| | methylpropylcarbamate | | | | |
| 19 | (3R,4S,5S,6R)-5-methoxy-4-((2R,3R)- | | 1.9 | | |
| | 2-methyl-3-(3-methylbut-2-enyl)oxiran- | | | | |
| | 2-yl)-1-oxaspiro[2.5]octan-6-yl (S)-1-(1- | | | | |
| | (((R)-1-(2,2-difluoroethyl)pyrrolidin-3- | | | | |
| | yl)methyl)-1H-imidazol-4-yl)-2- | | | | |
| | methylpropylcarbamate | | | | |
| 23 | (3R,4S,5S,6R)-5-methoxy-4-((2R,3R)- | | 5.1 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(1-(((R)-1-methylpyrrolidin-3- | | | | |
| | yl)methyl)-1H-pyrazol-4- | | | | |
| | yl)propylcarbamate | | | | |
| 25 | (3R,4S,5S,6R)-5-methoxy-4-((2R,3R)- | | 13 | | |
| | 2-methyl-3-(3-methylbut-2-enyl)oxiran- | | | | |
| | 2-yl)-1-oxaspiro[2.5]octan-6-yl (S)-1-(6- | | | | |
| | (3,3-difluoroazetidin-1-yl)pyridin-3-yl)- | | | | |
| | 2-methylpropylcarbamate | | | | |
| 26 | (3R,4S,5S,6R)-5-methoxy-4-((2R,3R)- | | 7.2 | | |
| | 2-methyl-3-(3-methylbut-2-enyl)oxiran- | | | | |
| | 2-yl)-1-oxaspiro[2.5]octan-6-yl (S)-2- | | | | |
| | methyl-1-(6-morpholinopyridin-3- | | | | |
| | yl)propylcarbamate | | | | |
| 40 | (3R,4S,5S,6R)-5-methoxy-4-((2R,3R)- | | 2.3 | | |
| | 2-methyl-3-(3-methylbut-2-enyl)oxiran- | | | | |
| | 2-yl)-1-oxaspiro[2.5]octan-6-yl ((S)-1- | | | | |
| | (1-isobutyl-1H-imidazol-4-yl)-2- | | | | |
| | methylpropyl)(methyl)carbamate | | | | |
| 41 | (3R,4S,5S,6R)-5-methoxy-4-((2R,3R)- | | 11.7 | | |
| | 2-methyl-3-(3-methylbut-2-enyl)oxiran- | | | | |
| | 2-yl)-1-oxaspiro[2.5]octan-6-yl ((S)-1- | | | | |
| | (6-(azetidin-1-yl)pyridin-3-yl)-2- | | | | |
| | methylpropyl)carbamate | | | | |
| 42 | (3R,4S,5S,6R)-5-methoxy-4-((2R,3R)- | | 10 | | |
| | 2-methyl-3-(3-methylbut-2-en-1- | | | | |
| | yl)oxiran-2-yl)-1-oxaspiro[2.5]octan-6- | | | | |
| | yl ((S)-1-(1-isobutyl-1H-imidazol-4-yl)- | | | | |
| | 2-methylpropyl)carbamate | | | | |
| compound | [(3R,6R,7S,8S)-7-methoxy-8-[(2R,3R)- | 18.0 | 20.0 | | |
| A | 2-methyl-3-(3-methylbut-2-enyl)oxiran- | | | | |
| | 2-yl]-2-oxaspiro[2.5]octan-6-yl] (E)-3- | | | | |
| | [4-[2- | | | | |
| | (dimethylamino)ethoxy]phenyl]prop-2- | | | | |
| | enoate | | | | |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound represented by Formula I: 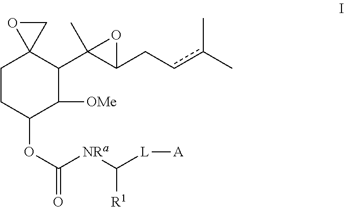
wherein: [symbol]
is a single or double bond;
R¹ is selected from the group consisting of hydrogen and C₁₋₆alkyl; wherein C₁₋₆alkyl may optionally be substituted by one, two, or more substituents each independently selected from the group consisting of halogen, cyano, hydroxyl, and N(R^{b}R^{c});
L is selected from the group consisting of a bond and —C(R^{L1}R^{L2})—;
A is a 5- or 6-membered monocyclic heteroaryl ring having one, two, or three heteroatoms each independently selected from O, S, or N; wherein A may be optionally substituted on an available carbon by a substituent independently selected for each occurrence from the group consisting of halogen, hydroxyl, C₁₋₆alkyl, C₁₋₆alkoxy, C₃₋₆cycloalkyl, phenyl, heterocyclyl, oxo, and —N(R^{b})(R^{c}); wherein C₁₋₆alkyl, C₁₋₆alkoxy, or C₃₋₆cycloalkyl may optionally be substituted by one, two, three or more substituents each independently selected from the group consisting of halogen, cyano, hydroxyl, and —N(R^{b})(R^{c}), and wherein A may be optionally substituted on an available nitrogen by R^{h};
R^{L1} and R^{L2} are each independently selected from the group consisting of hydrogen, halogen, hydroxyl, C₁₋₆alkyl, C₃₋₆cycloalkyl, and C₁₋₆alkoxy; wherein C₁₋₆alkyl, C₃₋₆cycloalkyl, and C₁₋₆alkoxy may optionally be substituted by one or more halogen atoms or a group selected from cyano or hydroxyl;
R^{a}, R^{b}, and R^{c} are each independently selected, for each occurrence, from the group consisting of hydrogen and C₁₋₆alkyl; wherein C₁₋₆alkyl may optionally be substituted by one or more substituents selected from the group consisting of halogen, cyano, oxo, and hydroxyl; or R^{b} and R^{c} may form, together with the nitrogen to which they are attached, a 4-6 membered heterocycle;
R^{h} is independently selected for each occurrence from the group consisting of hydrogen, C₁₋₆alkyl, C₂₋₆alkenyl, C₂₋₆alkynyl, C₃₋₆cycloalkyl, C₁₋₆alkyl-S(O)₂—, and C₁₋₆alkoxycarbonyl-, wherein C₁₋₆alkyl, C₂₋₆alkenyl, C₂₋₆alkynyl, C₃₋₆cycloalkyl, C₁₋₆alkyl-S(O)₂—, and C₁₋₆alkylcarbonyl- may optionally be substituted by one or more substituents selected from R^{P};
R^{P} is independently selected, for each occurrence, from the group consisting of halogen, hydroxyl, cyano, C₁₋₆alkoxy, and heterocyclyl; wherein heterocyclyl has one, two or three heteroatoms each independently selected from the group consisting of O, NR′ (wherein R′ is H or C₁₋₃alkyl) and S(O)_{w} (wherein w is 0, 1, or 2); wherein heterocyclyl may optionally be substituted by one, two or three substituents each independent selected from the group consisting of halogen, hydroxyl, and C₁₋₆alkyl; wherein C₁₋₆alkyl may optionally be substituted by one, two or three halogens;
or a pharmaceutically acceptable salt, stereoisomer, ester, or prodrug thereof.

2. The compound of claim 1, wherein A is substituted with C₁₋₂alkyl, oxo, or —N(R^{b})(R^{c}).

3. The compound of claim 1 or 2, wherein R^{h} is selected from the group consisting of hydrogen and C₁₋₆alkyl-R^{P}.

4. The compound of any one of claims 1-3, wherein R^{P} is selected from the group consisting 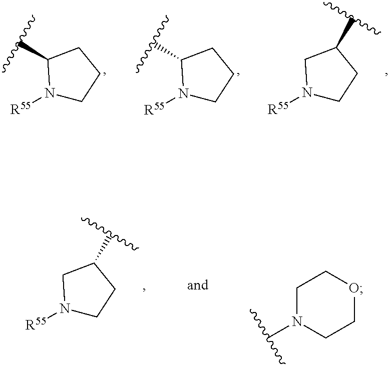
wherein R⁵⁵ is selected from the group consisting of hydrogen and C₁₋₂alkyl; wherein C₁₋₂alkyl may optionally be substituted by one or two halogens.

5. The compound of any one of claims 1-4, wherein R^{a} is hydrogen or methyl.

6. The compound of any one of claims 1-5, wherein L is selected from the group consisting of a bond, —CH₂—, —CHMe-, and —CMe₂-.

7. The compound of any one of claims 1-6, wherein L is a bond.

8. The compound of any one of claims 1-7, wherein R¹ is selected from the group consisting of hydrogen, methyl, ethyl, isopropyl, and sec-butyl.

9. The compound of any one of claims 1-8, wherein R¹ is isopropyl.

10. The compound of any one of claims 1-8, wherein R¹ is methyl.

11. The compound of any one of claims 1-10, wherein A is selected from the group consisting of: 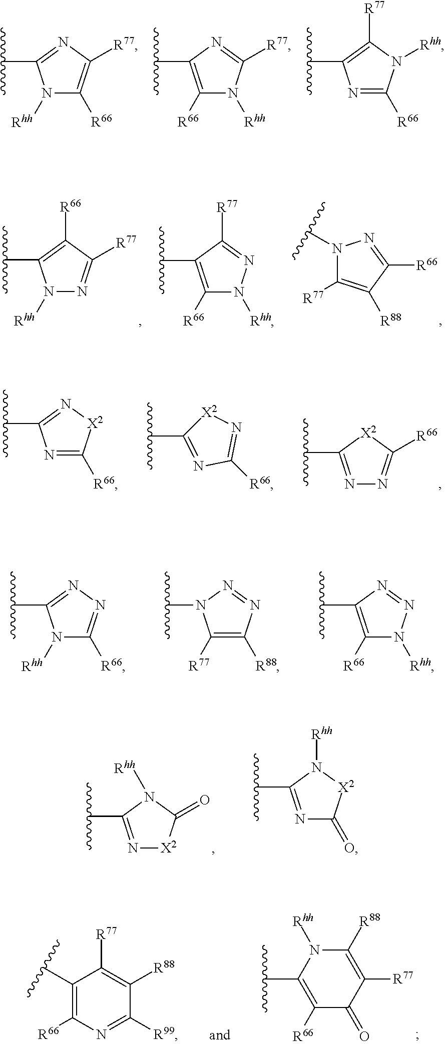
wherein X² independently for each occurrence is selected from the group consisting of O and S;
each R⁶⁶, R⁷⁷, R⁸⁸ and R⁹⁹ is independently selected for each occurrence from the group consisting of hydrogen, halogen, hydroxyl, C₁₋₂alkyl, heterocyclyl, and —NR^{b}R^{c}; wherein C₁₋₂alkyl may optionally be substituted by one, two or three substituents each independently selected from the group consisting of hydroxyl and halogen; and
R^{hh} is selected from the group consisting of hydrogen, C₁₋₆alkyl, and —C₁₋₃alkyl-heterocyclyl, wherein the heterocyclyl has one, two or three heteroatoms each independently selected from the group consisting of O, NR′ (wherein R′ is hydrogen or C₁₋₃alkyl) and S(O)_{w} (wherein w is 0, 1, or 2); and wherein heterocyclyl may optionally be substituted by a substituent selected from the group consisting of C₁₋₃alkyl (optionally substituted with 1-3 halogens), halogen, and hydroxyl.

12. The compound of any one of claims 1-11 wherein A is selected from the group consisting of: 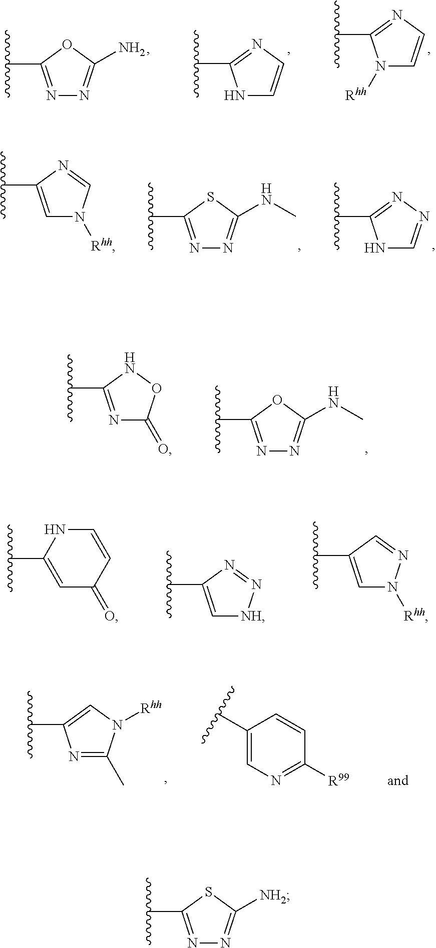
wherein
R^{hh} is hydrogen, C₁₋₆alkyl or —C₁₋₃alkyl-heterocyclyl, wherein heterocycyl may optionally be substituted by a substituent selected from the group consisting of C₁₋₃alkyl (optionally substituted by one, two, or three halogens), halogen, and hydroxyl;
and R⁹⁹ is heterocyclyl.

13. The compound of claim 11 or 12, wherein R^{hh} is selected from the group consisting of: 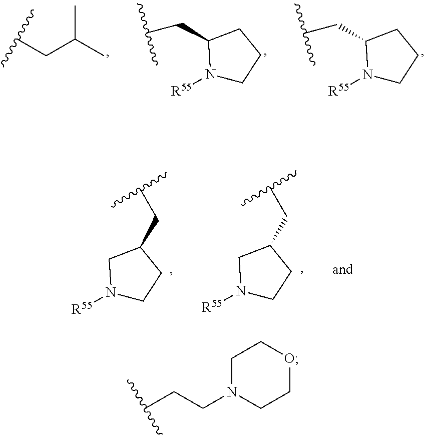
wherein R⁵⁵ is selected from the group consisting of hydrogen and C₁₋₂alkyl; wherein
C₁₋₂alkyl may optionally be substituted by 1 or 2 halogens.

14. The compound of claim 13, wherein R^{hh} is selected from the group consisting of:

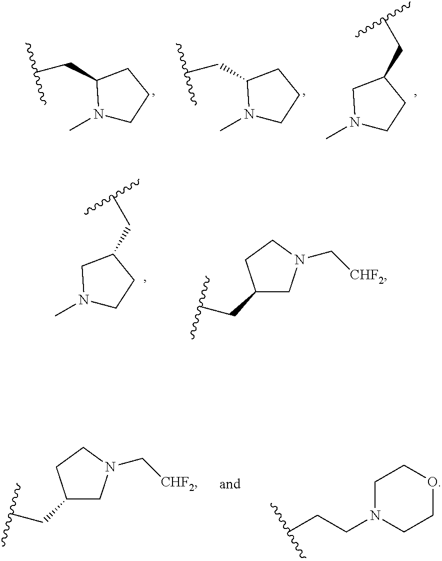

15. The compound of claim 12, wherein R⁹⁹ is selected from the group consisting of:

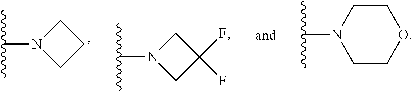

16. A compound represented by: 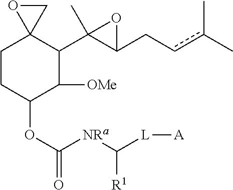
wherein: [symbol]
is a single or double bond;
R¹ is selected from the group consisting of hydrogen and C₁₋₆alkyl; wherein C₁₋₆alkyl may optionally be substituted by one, two, or more substituents each independently selected from the group consisting of halogen, cyano, hydroxyl, and N(R^{b}R^{c});
L is selected from the group consisting of a bond and —C(R^{L1}R^{L2})—;
A is selected from the group consisting of: 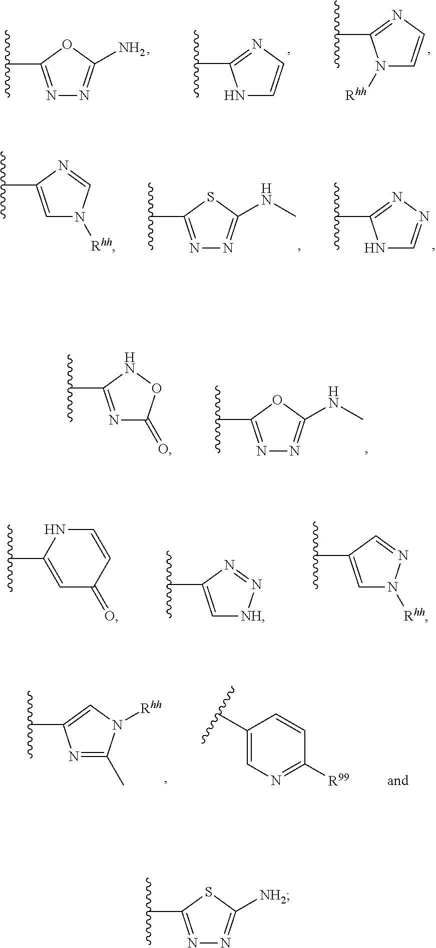
wherein
R^{hh} is hydrogen, C₁₋₆alkyl or —C₁₋₃alkyl-heterocyclyl, wherein heterocycyl may optionally be substituted by a substituent selected from the group consisting of C₁₋₃alkyl (optionally substituted by one, two, or three halogens), halogen, and hydroxyl;
R⁹⁹ is heterocyclyl;
R^{L1} and R^{L2} are each independently selected from the group consisting of hydrogen, halogen, hydroxyl, C₁₋₆alkyl, C₃₋₆cycloalkyl, and C₁₋₆alkoxy; wherein C₁₋₆alkyl, C₃₋₆cycloalkyl, and C₁₋₆alkoxy may optionally be substituted by one or more halogen atoms or a group selected from cyano or hydroxyl;
R^{a}, R^{b}, and R^{c} are each independently selected, for each occurrence, from the group consisting of hydrogen and C₁₋₆alkyl; wherein C₁₋₆alkyl may optionally be substituted by one or more substituents selected from the group consisting of halogen, cyano, oxo, and hydroxyl; or
R^{b} and R^{c} may form, together with the nitrogen to which they are attached, a 4-6 membered heterocycle;
R^{h} is independently selected for each occurrence from the group consisting of hydrogen, C₁₋₆alkyl, C₂₋₆alkenyl, C₂₋₆alkynyl, C₃₋₆cycloalkyl, C₁₋₆alkyl-S(O)₂—, and C₁₋₆alkoxycarbonyl-, wherein C₁₋₆alkyl, C₂₋₆alkenyl, C₂₋₆alkynyl, C₃₋₆cycloalkyl, C₁₋₆alkyl-S(O)₂—, and C₁₋₆alkylcarbonyl-may optionally be substituted by one or more substituents selected from R^{P};
R^{P} is independently selected, for each occurrence, from the group consisting of halogen, hydroxyl, cyano, C₁₋₆alkoxy, and heterocyclyl; wherein heterocyclyl has one, two or three heteroatoms each independently selected from the group consisting of O, NR′ (wherein R′ is H or C₁₋₃alkyl) and S(O)_{w} (wherein w is 0, 1, or 2); wherein heterocyclyl may optionally be substituted by one, two or three substituents each independent selected from the group consisting of halogen, hydroxyl, and C₁₋₆alkyl; wherein C₁₋₆alkyl may optionally be substituted by one, two or three halogens;
or a pharmaceutically acceptable salt, stereoisomer, ester, or prodrug thereof.

17. A compound selected from the group consisting of: (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-amino-1,3,4-oxadiazol-2-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(5-(methylamino)-1,3,4-oxadiazol-2-yl)propyl)carbamate; (3R,4S,5 S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-(methylamino)-1,3,4-oxadiazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1H-imidazol-2-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-2-yl)methyl)-1H-imidazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-2-yl)methyl)-1H-imidazol-2-yl)propyl)carbamate; (3R,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-(methylamino)-1,3,4-thiadiazol-2-yl)propyl)carbamate; (3R,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(5-(methylamino)-1,3,4-thiadiazol-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(4H-1,2,4-triazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(5-oxo-4,5-dihydro-1,2,4-oxadiazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(5-oxo-4,5-dihydro-1,2,4-oxadiazol-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-2-methyl-1-(1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(2-morpholinoethyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-(((S)-1-(2,2-difluoroethyl)pyrrolidin-3-yl)methyl)-1H-imidazol-4-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-(((R)-1-(2,2-difluoroethyl)pyrrolidin-3-yl)methyl)-1H-imidazol-4-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(4-oxo-1,4-dihydropyridin-2-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1H-1,2,3-triazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((S)-1-methylpyrrolidin-3-yl)methyl)-1H-pyrazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(1-(((R)-1-methylpyrrolidin-3-yl)methyl)-1H-pyrazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(2-methyl-1-(((S)-1-methylpyrrolidin-3-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(6-(3,3-difluoroazetidin-1-yl)pyridin-3-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-2-methyl-1-(6-morpholinopyridin-3-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-3-methyl-1-(5-oxo-4,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-oxo-4,5-dihydro-1,2,4-oxadiazol-3-yl)propan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-oxo-4,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-oxo-2,5-dihydro-1,2,4-oxadiazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(4H-1,2,4-triazol-3-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-1-(5-amino-1,3,4-oxadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-(methylamino)-1,3,4-oxadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-3-methyl-1-(5-(methylamino)-1,3,4-thiadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-3-methyl-1-(5-(methylamino)-1,3,4-thiadiazol-2-yl)butan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((R)-1-(5-amino-1,3,4-thiadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(5-amino-1,3,4-thiadiazol-2-yl)-3-methylbutan-2-yl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl (2-methyl-2-(1-(((R)-1-methylpyrrolidin-2-yl)methyl)-1H-imidazol-4-yl)propyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl (2-methyl-2-(4H-1,2,4-triazol-3-yl)propyl)carbamate; (3R,4S,5S,6R)-5-methoxy-4-((2R,3R)-2-methyl-3-(3-methylbut-2-enyl)oxiran-2-yl)-1-oxaspiro[2.5]octan-6-yl ((S)-1-(1-isobutyl-1H-imidazol-4-yl)-2-methylpropyl)(methyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-1-(6-(azetidin-1-yl)pyridin-3-yl)-2-methylpropyl)carbamate; (3R,4S,5S,6R)-5-methoxy-4-((2R,3R)-2-methyl-3-(3-methylbut-2-en-1-yl)oxiran-2-yl)-1-oxaspiro[2.5]octan-6-yl((S)-1-(1-isobutyl-1H-imidazol-4-yl)-2-methylpropyl)carbamate; and a pharmaceutically acceptable salt or stereoisomer thereof.

18. A pharmaceutically acceptable composition comprising a compound of any one of claims 1-17 and a pharmaceutically acceptable excipient.

19. The composition of claim 18, wherein the composition is formulated as a unit dose.

20. The composition of claim 18, wherein the composition is formulated for subcutaneous administration.

21. The composition of claim 18, wherein the composition is formulated for intravenous administration.

22. A method of treating and/or controlling obesity, comprising administering to a patient in need thereof an effective amount of a compound of any one of claims 1-17.

23. A method of inducing weight loss in a patient in need thereof, comprising administering to said patient an effective amount of a compound of any one of claims 1-17.

24. A method of substantially preventing weight gain in a patient in need thereof, comprising administering to said patient an effective amount of a compound of any one of claims 1-17.

25. The method of any one of claims 22-24, wherein the patient is a human.

26. The method of any one of claims 22-24, wherein the patient is a cat or dog.

27. The method of any one of claims 22-24, wherein the patient has a body mass index greater than or equal to about 30 kg/m² before the administration.

28. The method of any one of claims 22-27, wherein administering comprises subcutaneous administration.

29. The method of any one of claims 22-27, wherein administering comprises intravenous administration.

30. The method of any one of claims 22-29, wherein administering comprises once, twice, or thrice weekly administration.

31. The method of any one of claims 22-30, comprising administering said compound in an amount sufficient to establish inhibition of intracellular MetAP2 effective to increase thioredoxin production in the patient and to induce multi organ stimulation of anti-obesity processes in the subject.

32. The method of claim 31, comprising administering said compound in an amount insufficient to reduce angiogenesis in the patient.
